(19) United States
(12) Patent Application Publication
(10) Pub. No.: US 2026/0223593 A1
(43) Pub. Date: Jul. 30, 2026

(54) Organic Compound, Light-Emitting Device, Display Apparatus, And Electronic Appliance

(71) Applicant: Semiconductor Energy Laboratory Co., Ltd., Kanagawa-ken (JP)

(72) Inventors: Yuko KUBOTA, Atsugi, Kanagawa (JP); Takeyoshi WATABE, Atsugi, Kanagawa (JP); Hiromi SEO, Sagamihara, Kanagawa (JP); Nobuharu OHSAWA, Zama, Kanagawa (JP); Yui YOSHIYASU, Atsugi, Kanagawa (JP); Sachiko KAWAKAMI, Atsugi, Kanagawa (JP)

(73) Assignee: Semiconductor Energy Laboratory Co., Ltd., Kanagawa-ken (JP)

(21) Appl. No.: 19/157,894

(22) PCT Filed: Feb. 16, 2024
(86) PCT No.: PCT/IB2024/051457

§ 371 Date: Aug. 19, 2025

(30) Foreign Application Priority Data
Feb. 24, 2023 (JP) 2023-027381

(51) Int. Cl. H10K 85/60; C07D 487/04; H10K 50/17; H10K 59/12

(52) U.S. Cl. CPC H10K 85/6572; C07D 487/04; H10K 50/171; H10K 59/12

(57) ABSTRACT

An organic compound having an electron-injection property and low water solubility is provided. An organic compound represented by General Formula (G1) below is provided. In General Formula (G1), Ar is an aromatic skeleton represented by General Formula (Ar-1); L represents an alkylene group, an arylene group, or a heteroarylene group; n represents an integer greater than or equal to 0 and less than or equal to 3; m represents an integer greater than or equal to 1 and less than or equal to 6; and each of R1 to R12 independently represents hydrogen or an alkyl group. In General Formula (Ar-1), a ring A represents a benzene ring or a naphthalene ring; each of Ar1 to Ar3 independently represents an aryl group or a heteroaryl group, and any m carbon atoms contained in the ring A and Ar1 to Ar3 include m bonds in General Formula (G1); k represents an integer greater than or equal to 1 and less than or equal to 3; and p represents an integer greater than or equal to 2 and less than or equal to 6.

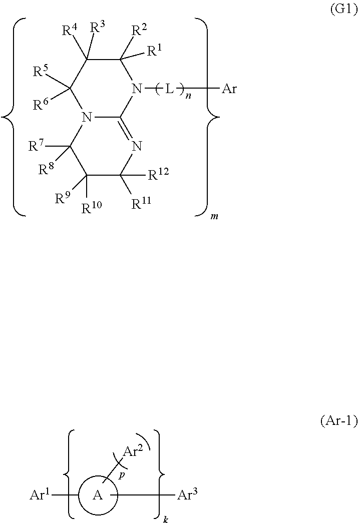

### TECHNICAL FIELD

One embodiment of the present invention relates to an organic compound, a light-emitting device, a light-emitting apparatus, a light-emitting and light-receiving apparatus, a display apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Accordingly, more specific examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### BACKGROUND ART

Recent display apparatuses have been expected to be applied to a variety of uses. Usage examples of large-sized display apparatuses include a television device for home use (also referred to as TV or television receiver), digital signage, and a PID (Public Information Display). In addition, a smartphone and a tablet terminal each including a touch panel, and the like, are being developed as portable information terminals.

Furthermore, higher-resolution display apparatuses have been required. As devices requiring high-definition display apparatuses, for example, devices for virtual reality (VR), augmented reality (AR), substitutional reality (SR), and mixed reality (MR) have been actively developed.

Light-emitting apparatuses including light-emitting devices (also referred to as light-emitting elements) have been developed as display apparatuses, for example. Light-emitting devices utilizing an electroluminescence (hereinafter referred to as EL) phenomenon (also referred to as EL devices or EL elements) have features such as ease of reduction in thickness and weight, high-speed response to input signals, and driving with a constant DC voltage power source, and have been used in display apparatuses.

Patent Document 1 discloses a display apparatus using an organic EL device (also referred to as organic EL element) for VR. Patent Document 2 discloses a light-emitting device with a low driving voltage and favorable reliability in which a mixed film of a transition metal and an organic compound including an unshared electron pair is used as an electron-injection layer.

### REFERENCES

### Patent Documents

[Patent Document 1] PCT International Publication No. 2018/087625
[Patent Document 2] Japanese Published Patent Application No. 2018-201012

### SUMMARY OF THE INVENTION

### Problems to be Solved by the Invention

As a method for forming an organic semiconductor film in a predetermined shape, a vacuum evaporation method using a metal mask (mask vapor deposition) is widely used. However, in these days of higher density and higher resolution, mask vapor deposition has come close to the limit of increasing the resolution for various reasons such as the alignment accuracy and the distance between the mask and the substrate. Meanwhile, shape processing of an organic semiconductor film by a lithography method enables formation of a finer pattern. The processing of an organic semiconductor film by a lithography method can also achieve an increase in area easily and thus has been actively researched.

An organic EL device includes an organic compound layer containing a light-emitting substance (corresponding to the above organic semiconductor film) between electrodes (between a first electrode and a second electrode), and energy generated by recombination of carriers (holes and electrons) injected to the organic compound layer from the electrodes causes light emission.

Carrier injection, especially electron injection into the organic compound layer, through which electricity is difficult to flow, has to overcome a high energy barrier and therefore essentially requires a high voltage. In view of this, currently, an electron-injection layer in contact with a cathode contains a donor substance (also referred to as an electron donor), whereby a reduction in voltage is achieved. Specific examples of the donor substance include, typically, alkali metals such as lithium (Li), which have a low work function, and compounds of the alkali metals.

However, in the case where the above-described lithography method is employed to manufacture a light-emitting device including an organic compound layer containing a donor substance, there has been a problem in that oxygen or water in the air and a chemical solution or water in the process cause a significant increase in driving voltage or a marked reduction in current efficiency.

A way of solving the problem is to perform processing by a lithography method halfway through a process of forming the organic compound layer of the light-emitting device (that is, before forming the layer containing a donor substance). In other words, processing of the organic compound layer by a lithography method is performed prior to the formation of the electron-injection layer, and then the step of forming the electron-injection layer and the subsequent steps are performed, whereby degradation of characteristics can be avoided.

However, for a tandem light-emitting device, the above solving way cannot be employed and the processing of the organic compound layer by a lithography method has inevitably caused a significant degradation of characteristics.

This is because the tandem light-emitting device includes an organic compound layer with a structure where a plurality of light-emitting layers are stacked in series with an intermediate layer (also referred to as charge generation layer) therebetween, and the intermediate layer contains a donor substance so that electrons can be injected into a light-emitting layer on the anode side. Since the intermediate layer is provided between two light-emitting layers, when the organic compound layer including the two light-emitting layers is processed by a lithography method, the intermediate layer is also processed inevitably by a lithography method and is consequently exposed to oxygen or water in the air or a chemical solution or water in the process.

Like exposure of the electron-injection layer to a processing step by a lithography method, the exposure of the layer containing a donor substance in the intermediate layer to a processing step by a lithography method has caused a significant increase in driving voltage and a marked reduction in current efficiency of the light-emitting device.

Another way of solving the above problem is using an organic compound having an electron-injection property, instead of a donor substance, for the electron-injection layer or the intermediate layer. That is, in the above way, the organic compound layer containing no donor substance is processed by a lithography method, so that the degradation of characteristics of the light-emitting device due to a donor substance can be avoided.

However, if the water solubility of the organic compound is high, the layer including the organic compound is dissolved when exposed to water or a chemical solution containing water as a solvent, which might cause degraded characteristics, a shape defect, or the like.

An object of one embodiment of the present invention is to provide an organic compound having an electron-injection property. Another object of one embodiment of the present invention is to provide an organic compound having low water solubility. Another object of one embodiment of the present invention is to provide an organic compound having a high glass transition temperature (Tg). Another object of one embodiment of the present invention is to provide a light-emitting device having favorable characteristics. Another object of one embodiment of the present invention is to provide a novel organic compound or a novel light-emitting device.

Another object of one embodiment of the present invention is to provide a display apparatus having high display quality. Another object of one embodiment of the present invention is to provide a high-resolution display apparatus. Another object of one embodiment of the present invention is to provide a high-definition display apparatus. Another object of one embodiment of the present invention is to provide a display apparatus having a high aperture ratio. Another object of one embodiment of the present invention is to provide a highly reliable display apparatus. Another object of one embodiment of the present invention is to provide a novel display apparatus that is highly convenient, useful, or reliable. Another object of one embodiment of the present invention is to provide a novel electronic appliance that is highly convenient, useful, or reliable. Another object of one embodiment of the present invention is to provide a novel display apparatus or a novel electronic appliance.

Note that the description of these objects does not preclude the existence of other objects. One embodiment of the present invention does not necessarily achieve all these objects. Other objects will be apparent from the description of the specification, the drawings, the claims, and the like, and other objects can be derived from the description of the specification, the drawings, the claims, and the like.

### Means for Solving the Problems

To solve the above problems, one embodiment of the present invention provides an organic compound having a bicyclic guanidine skeleton and an aromatic skeleton. Such an organic compound has high basicity and an electron-injection property, and thus can be used, instead of a donor substance, for an electron-injection layer or an intermediate layer in an organic compound layer of a light-emitting device. Thus, as compared with the case of using a donor substance, even when the organic compound layer is exposed to water, oxygen, or the like in a processing step by a lithography method, a significant increase in driving voltage and a marked reduction in current efficiency of the light-emitting device can be prevented, whereby the light-emitting device can have favorable characteristics.

Furthermore, the present inventors have found that increasing the hydrophobic property of the aromatic skeleton in the organic compound can reduce the water solubility of the organic compound.

Thus, one embodiment of the present invention is an organic compound represented by General Formula (G1) below.

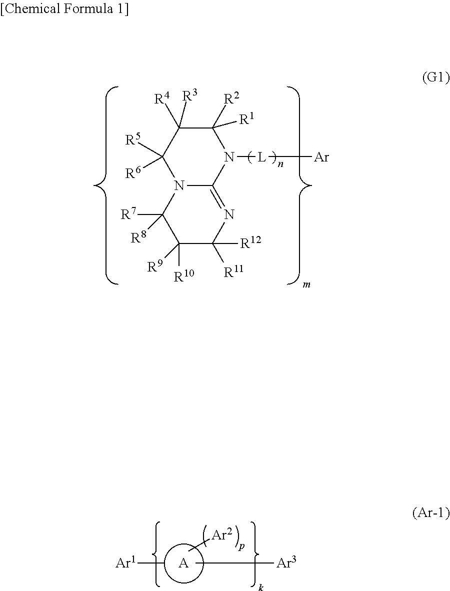

Note that in General Formula (G1), Ar is an aromatic skeleton represented by General Formula (Ar-1); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; m represents an integer greater than or equal to 1 and less than or equal to 6; and each of R¹ to R¹² independently represents hydrogen (including deuterium) or an alkyl group having 1 to 10 carbon atoms. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other. In General Formula (Ar-1), a ring A represents a benzene ring or a naphthalene ring; each of Ar¹ to Ar³ independently represents a substituted or unsubstituted aryl group having 6 to 30 carbon atoms or a substituted or unsubstituted heteroaryl group having 1 to 30 carbon atoms, and any m carbon atoms contained in the ring A and Ar¹ to Ar³ include m bonds in General Formula (G1); k represents an integer greater than or equal to 1 and less than or equal to 3; and p represents an integer greater than or equal to 2 and less than or equal to 6. Note that a plurality of Ar²s may be the same or different from each other. In the case where k is greater than or equal to 2, p's may be the same or different from each other, the rings A may be the same or different from each other, and Ar²s may be the same or different from each other.

Another embodiment of the present invention is an organic compound represented by General Formula (G1) below.

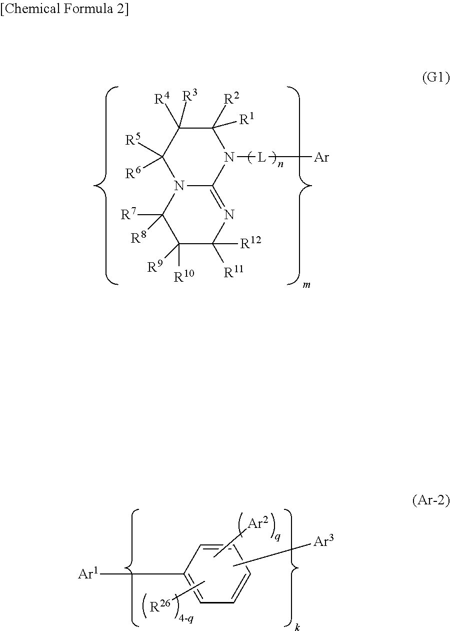

Note that in General Formula (G1), Ar is an aromatic skeleton represented by General Formula (Ar-2); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; m represents an integer greater than or equal to 1 and less than or equal to 6; and each of R¹ to R¹² independently represents hydrogen (including deuterium) or an alkyl group having 1 to 10 carbon atoms. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other. In General Formula (Ar-2), each of Ar¹ to Ar³ independently represents a substituted or unsubstituted aryl group having 6 to 30 carbon atoms or a substituted or unsubstituted heteroaryl group having 1 to 30 carbon atoms, and any m carbon atoms contained in Ar¹ to Ar³ include m bonds in General Formula (G1); R²⁶ represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms; k represents an integer greater than or equal to 1 and less than or equal to 3, and q represents an integer greater than or equal to 2 and less than or equal to 4. Note that a plurality of Ar²s may be the same or different from each other. In the case where k is greater than or equal to 2, p's may be the same or different from each other, R²⁶s may be the same or different from each other, and Ar²s may be the same or different from each other.

Another embodiment of the present invention is an organic compound represented by General Formula (G1) below.

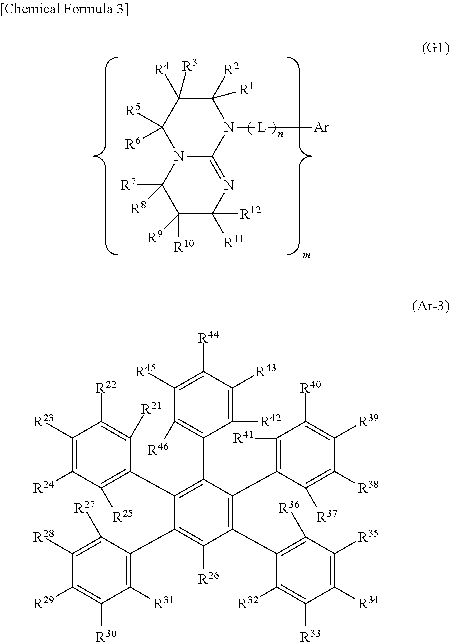

Note that in General Formula (G1), Ar is an aromatic skeleton represented by General Formula (Ar-3); L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; m represents an integer greater than or equal to 1 and less than or equal to 6; and each of R¹ to R¹² independently represents hydrogen (including deuterium) or an alkyl group having 1 to 10 carbon atoms. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other. In General Formula (Ar-3), any m groups of R²¹ to R⁴⁶ represent m bonding positions in General Formula (G1); and each of the other groups of R²¹ to R⁴⁶ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

Another embodiment of the present invention is an organic compound represented by General Formula (G2) below.

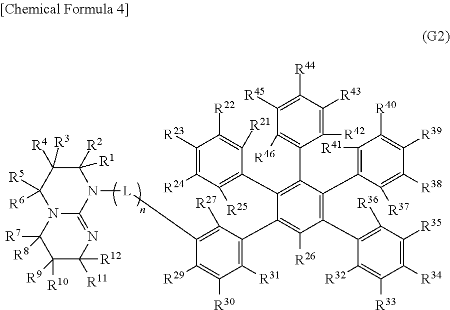

Note that in General Formula (G2), L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; and each of R¹ to R¹² independently represents hydrogen (including deuterium) or an alkyl group having 1 to 10 carbon atoms. In the case where n is greater than or equal to 2, Ls may be the same or different from each other. Each of R²¹ to R²⁷ and R²⁹ to R⁴⁶ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

Another embodiment of the present invention is an organic compound represented by Structural Formula (100) below.

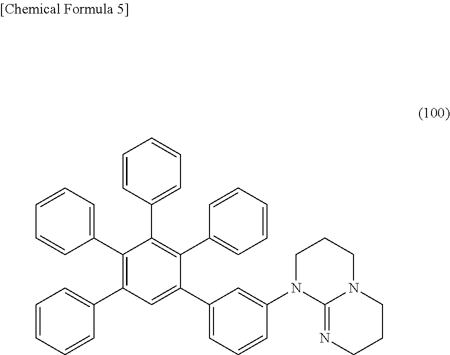 [SMILES: c1ccc(-c2cc(-c3cccc(N4CCCN5CCCN=C54)c3)c(-c3ccccc3)c(-c3ccccc3)c2-c2ccccc2)cc1]

Another embodiment of the present invention is a light-emitting device using the organic compound having any of the above structures.

Another embodiment of the present invention is a display apparatus including the light-emitting device having any of the above structures, and a transistor or a substrate.

Another embodiment of the present invention is an electronic appliance including the display apparatus having the above structure, and a sensor portion, an input portion, or a communication portion.

Note that the display apparatus in this specification includes, in its category, an image display apparatus that uses a light-emitting device. The display apparatus may also include a module in which a light-emitting device over a substrate is provided with a connector such as an anisotropic conductive film or a TCP (Tape Carrier Package), a module in which a printed wiring board is provided at the end of a TCP, and a module in which an IC (integrated circuit) is directly mounted on a light-emitting device by a COG (Chip On Glass) method. Furthermore, a lighting device or the like may include the display apparatus.

### Effect of the Invention

One embodiment of the present invention can provide an organic compound having an electron-injection property. Another embodiment of the present invention can provide an organic compound having low water solubility. Another embodiment of the present invention can provide an organic compound having a high glass transition temperature (Tg). Another embodiment of the present invention can provide a light-emitting device having favorable characteristics. Another embodiment of the present invention can provide a novel organic compound or a novel light-emitting device.

One embodiment of the present invention can provide a display apparatus having high display quality. Another embodiment of the present invention can provide a high-resolution display apparatus. Another embodiment of the present invention can provide a high-definition display apparatus. Another embodiment of the present invention can provide a display apparatus having a high aperture ratio. Another embodiment of the present invention can provide a highly reliable display apparatus. Another embodiment of the present invention can provide a novel display apparatus that is highly convenient, useful, or reliable. Another embodiment of the present invention can provide a novel electronic appliance that is highly convenient, useful, or reliable. Another embodiment of the present invention can provide a novel display apparatus or a novel electronic appliance.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIG. 1A to FIG. 1C are diagrams illustrating light-emitting devices.

FIG. 2 is a diagram illustrating a light-emitting device.

FIG. 3A and FIG. 3B are a top view and a cross-sectional view of a display apparatus.

FIG. 4A to FIG. 4D are diagrams illustrating a light-emitting device.

FIG. 5A to FIG. 5E are cross-sectional views illustrating an example of a method for manufacturing a display apparatus.

FIG. 6A to FIG. 6D are cross-sectional views illustrating the example of the method for manufacturing the display apparatus.

FIG. 7A to FIG. 7D are cross-sectional views illustrating the example of the method for manufacturing the display apparatus.

FIG. 8A to FIG. 8C are cross-sectional views illustrating the example of the method for manufacturing the display apparatus.

FIG. 9A to FIG. 9C are cross-sectional views illustrating the example of the method for manufacturing the display apparatus.

FIG. 10A to FIG. 10C are cross-sectional views illustrating the example of the method for manufacturing the display apparatus.

FIG. 11A to FIG. 11C are diagrams illustrating a conventional structure.

FIG. 12A to FIG. 12E are diagrams illustrating a method for processing a film.

FIG. 13A to FIG. 13E are diagrams illustrating a method for processing a film.

FIG. 14A and FIG. 14B are perspective views illustrating a structure example of a display module.

FIG. 15A and FIG. 15B are cross-sectional views illustrating structure examples of a display apparatus.

FIG. 16A to FIG. 16D are diagrams illustrating examples of electronic appliances.

FIG. 17A to FIG. 17F are diagrams illustrating examples of electronic appliances.

FIG. 18A to FIG. 18C are ¹H NMR spectra of Ph3TP-hpp.

FIG. 19 shows the luminance-current density characteristics of a light-emitting device 1.

FIG. 20 shows the luminance-voltage characteristics of the light-emitting device 1.

FIG. 21 shows the current efficiency-luminance characteristics of the light-emitting device 1.

FIG. 22 shows the current density-voltage characteristics of the light-emitting device 1.

FIG. 23 shows the electroluminescence spectrum of the light-emitting device 1.

FIG. 24 shows a luminance change over driving time of the light-emitting device 1.

### MODE FOR CARRYING OUT THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

Note that in structures of the invention described below, the same portions or portions having similar functions are denoted by the same reference numerals in different drawings, and the description thereof is not repeated. Furthermore, the same hatch pattern is used for the portions having similar functions, and the portions are not especially denoted by reference numerals in some cases.

The position, size, range, and the like of each component illustrated in drawings do not represent the actual position, size, range, and the like in some cases for easy understanding. Thus, the disclosed invention is not necessarily limited to the position, size, range, and the like disclosed in the drawings.

Note that the term “film” and the term “layer” can be used interchangeably depending on the case or the circumstances. For example, the term “conductive layer” can be replaced with the term “conductive film”. For another example, the term “insulating film” can be replaced with the term “insulating layer”.

Note that in this specification and the like, a device manufactured using a metal mask or an FMM (a fine metal mask, a high-resolution metal mask) may be referred to as a device having an MM (a metal mask) structure. In addition, in this specification and the like, a device manufactured without using a metal mask or an FMM is sometimes referred to as a device having an MML (metal maskless) structure.

In this specification and the like, a light-emitting device (also referred to as a light-emitting element) includes an EL layer between a pair of electrodes. The EL layer includes at least a light-emitting layer.

Note that in this specification and the like, a tapered shape indicates a shape in which at least part of a side surface of a component is inclined to a substrate surface. For example, a tapered shape preferably includes a region where an angle formed between the inclined side surface and the substrate surface (such an angle is also referred to as a taper angle) is less than 90°. Note that the side surface of the component and the substrate surface are not necessarily completely flat and may be substantially flat with a slight curvature or substantially flat with slight unevenness.

Note that in this specification, an aromatic ring refers to a cyclic structure included in an aromatic hydrocarbon or a heteroaromatic compound. A cyclic structure included in a heteroaromatic compound is particularly referred to as a heteroaromatic ring in some cases. An aromatic skeleton refers to a single aromatic ring or a skeleton in which a plurality of aromatic rings are connected by a single bond.

### Embodiment 1

In this embodiment, an organic compound of one embodiment of the present invention is described.

One embodiment of the present invention provides an organic compound having a bicyclic guanidine skeleton and an aromatic skeleton. Such an organic compound has high basicity and an electron-injection property, and thus can be used, instead of a donor substance, for an electron-injection layer or an intermediate layer in an organic compound layer of a light-emitting device. Thus, as compared with the case of using a donor substance, even when the organic compound layer is exposed to water, oxygen, or the like in a processing step by a lithography method, a significant increase in driving voltage and a marked reduction in current efficiency of the light-emitting device can be prevented, whereby the light-emitting device can have favorable characteristics.

However, an organic compound having high basicity has a large dipole moment and accordingly has high water solubility. This allows, for example, dissolution of a layer including the organic compound or permeation of a chemical solution into the layer including the organic compound in a processing step by a lithography method involving exposure to water or the chemical solution containing water as a solvent, which might cause degraded characteristics, a shape defect, or the like of a light-emitting device. Thus, the water solubility needs to be reduced while the electron-injection property sufficient for use in a light-emitting device is maintained. One possible way to reduce the water solubility is increasing the hydrophobic property of an aromatic skeleton.

Since a bicyclic guanidine skeleton has high planarity, an organic compound having the skeleton is likely to be crystallized. Thus, the use of the organic compound having the bicyclic guanidine skeleton for a light-emitting device might decrease the reliability of the light-emitting device. One possible solution is to use a polysubstituted aromatic skeleton as the aromatic skeleton to cause a twist in the aromatic skeleton.

Thus, an organic compound of one embodiment of the present invention is an organic compound represented by General Formula (G1) below.

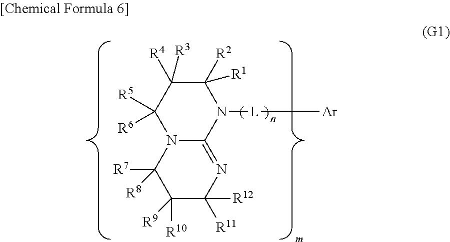

In General Formula (G1) above, Ar is a polysubstituted aromatic skeleton; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; m represents an integer greater than or equal to 1 and less than or equal to 6; and each of R¹ to R¹² independently represents hydrogen (including deuterium) or an alkyl group having 1 to 10 carbon atoms. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other.

A polysubstituted aromatic skeleton has a high molecular weight and thus can reduce the water solubility of the organic compound. This accordingly can avoid dissolution of the layer including the organic compound in a processing step by a lithography method.

A substituent of the polysubstituted aromatic skeleton is preferably an aryl group or a heteroaryl group that is sterically bulky. A twist is caused in the skeleton in this case, so that the planarity of the whole organic compound can be decreased and the crystallinity of the layer including the organic compound can be decreased. Accordingly, the glass transition temperature (T_{g}) of the organic compound can be increased.

In addition, owing to the twist caused in the skeleton, expansion of a conjugated system can be suppressed in the polysubstituted aromatic skeleton. This increases the HOMO-LUMO energy gap and inhibits injection of holes and electrons into the organic compound. Thus, in the light-emitting device whose carrier-transport layer has a structure where a carrier-transport material and the organic compound are mixed, recombination of holes and electrons can be inhibited from occurring in the carrier-transport layer, so that loss of excitons can be inhibited. Thus, the emission efficiency of the light-emitting device can be increased.

In General Formula (G1), in the case where n is 0, the organic compound can be easily synthesized. In the case where n is an integer greater than or equal to 1 and less than or equal to 3, as compared with the case where n is 0, the number of hydrophobic substituents (an alkylene group having 1 to 10 carbon atoms, a substituted or unsubstituted arylene group having 6 to 30 carbon atoms, or a substituted or unsubstituted heteroarylene group having 1 to 11 carbon atoms) increases and the molecular weight of the organic compound increases, whereby the water solubility can be further reduced.

In General Formula (G1), in the case where m is an integer greater than or equal to 2 and less than or equal to 6, the basicity of the organic compound can be increased. In the case where m is 1, the water solubility of the organic compound can be reduced.

As the polysubstituted aromatic skeleton in General Formula (G1), a skeleton represented by General Formula (Ar-1) below can be used, for example.

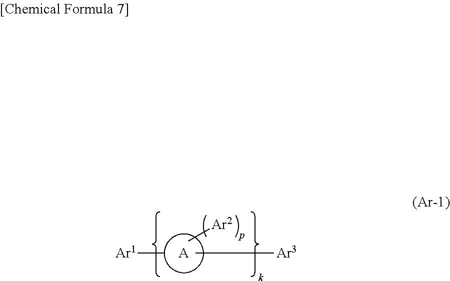

In General Formula (Ar-1), a ring A represents a benzene ring or a naphthalene ring; each of Ar¹ to Ar³ independently represents a substituted or unsubstituted aryl group having 6 to 30 carbon atoms or a substituted or unsubstituted heteroaryl group having 1 to 30 carbon atoms; any m carbon atoms contained in the ring A and Ar¹ to Ar³ include m bonds in General Formula (G1); k represents an integer greater than or equal to 1 and less than or equal to 3; and p represents an integer greater than or equal to 2 and less than or equal to 6. Note that a plurality of Ar²s may be the same or different from each other. In the case where k is greater than or equal to 2, p's may be the same or different from each other, the rings A may be the same or different from each other, and Ar²s may be the same or different from each other.

In General Formula (Ar-1), in the case where the ring A includes a substituent in addition to Ar¹, Ar², and Ar³, specific examples of the substituent include an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms.

A skeleton represented by General Formula (Ar-1) has a large molecular weight because of its structure where five or more aromatic rings are bonded by a single bond; thus, the water solubility of an organic compound can be reduced. This accordingly can avoid dissolution of the layer including the organic compound in a processing step by a lithography method.

The skeleton represented by General Formula (Ar-1) includes one Ar¹, p Ar²s, and one Ar³ as substituents of the ring A (a substituted or unsubstituted benzene ring or a substituted or unsubstituted naphthalene ring); thus, the periphery of the ring A is sterically congested. In general, when a plurality of aromatic rings are connected to each other by a single bond, a π-electron conjugated system of the aromatic rings expand; thus, the aromatic rings are arranged on the same plane. On the other hand, in the skeleton represented by General Formula (Ar-1) where the periphery of the ring A is sterically congested and a twist is caused, the conjugated system is less likely to expand. This inhibits the ring A, Ar¹, Ar², and Ar³ to be arranged on the same plane, thereby decreasing the planarity of the whole organic compound and decreasing the crystallinity of a layer including the organic compound. This can accordingly increase the T_{g} of the organic compound, thereby preventing abnormality in film quality of the layer including the organic compound in a heating step.

In some cases, a film including an organic compound with a low T_{g} has unstable film quality and might cause decrease in reliability of a light-emitting device. However, the use of the aromatic skeleton represented by General Formula (Ar-1) can increase the T_{g} of the organic compound. Thus, the use of the organic compound can prevent abnormality in film quality of the layer including the organic compound in a heating step and can improve the reliability of the light-emitting device.

Note that the use of a ring in which three or more aromatic rings are condensed as the ring A is not preferable because the density of the substituents of the ring A is decreased in some cases. The use of a heteroaromatic ring as the ring A is also not preferable because the number of substituents that can be introduced into the ring A is decreased in some cases. In General Formula (Ar-1) of one embodiment of the present invention, a benzene ring or a naphthalene ring is used as the ring A; thus, the density of the substituents of the ring A can be increased, a twist in the polysubstituted aromatic skeleton can be intensified, and the planarity of the organic compound can be decreased. Furthermore, expansion of the conjugated system can be suppressed, and the HOMO-LUMO energy gap can be increased.

Although the use of a ring in which three or more aromatic rings are condensed as the ring A might increase the visible-light-absorption intensity of the organic compound, a benzene ring or a naphthalene ring is used as the ring A in General Formula (Ar-1); thus, the increase in the visible-light-absorption intensity of the organic compound can be suppressed and a decrease in the emission efficiency of the light-emitting device can be prevented.

It is further preferable to use a substituted or unsubstituted benzene ring as the ring A. In that case, the increase in the visible-light-absorption intensity of the organic compound can be further suppressed and the decrease in the emission efficiency of the light-emitting device can be further prevented.

Note that p is further preferably an integer greater than or equal to 3 and less than or equal to 6. The number of substituents of the ring A is increased as compared with the case where p is 2, so that steric hindrance around the ring A can be intensified and the planarity of the molecule can be decreased. Note that in the case where a substituted or unsubstituted benzene ring is used as the ring A, p is an integer greater than or equal to 2 and less than or equal to 4.

Alternatively, as the polysubstituted aromatic skeleton in General Formula (G1), an aromatic skeleton represented by General Formula (Ar-2) below can be used, for example. Note that General Formula (Ar-2) shows a structure where the ring A in General Formula (Ar-1) is limited to a substituted or unsubstituted benzene ring. Thus, the description of the structure, effect, and the like of General Formula (Ar-1) can also be applied to General Formula (Ar-2).

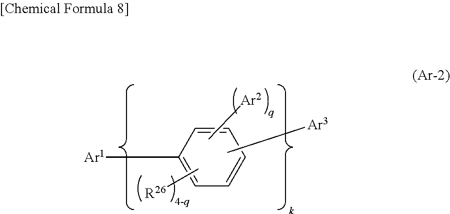

In General Formula (Ar-2), each of Ar¹ to Ar³ independently represents a substituted or unsubstituted aryl group having 6 to 30 carbon atoms or a substituted or unsubstituted heteroaryl group having 1 to 30 carbon atoms, and any m carbon atoms contained in Ar¹ to Ar³ include m bonds in General Formula (G1); R²⁶ represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms; k represents an integer greater than or equal to 1 and less than or equal to 3; and q represents an integer greater than or equal to 2 and less than or equal to 4. Note that a plurality of Ar²s may be the same or different from each other. In the case where k is greater than or equal to 2, p's may be the same or different from each other, R²⁶s may be the same or different from each other, and Ar²s may be the same or different from each other.

As described above, General Formula (Ar-2) has a structure where the ring A in General Formula (Ar-1) is limited to a substituted or unsubstituted benzene ring. In that case, the increase in the visible-light-absorption intensity of the organic compound can be further suppressed and the decrease in the emission efficiency of the light-emitting device can be further prevented.

Alternatively, as the polysubstituted aromatic skeleton in General Formula (G1), an aromatic skeleton represented by General Formula (Ar-3) below can be used, for example. Note that General Formula (Ar-3) shows a structure where Ar¹, Ar², and Ar³ in General Formula (Ar-2) are limited to substituted or unsubstituted benzene rings and p is limited to 3. Thus, the description of the structure, effect, and the like of General Formulae (Ar-1) and (Ar-2) can also be applied to General Formula (Ar-3).

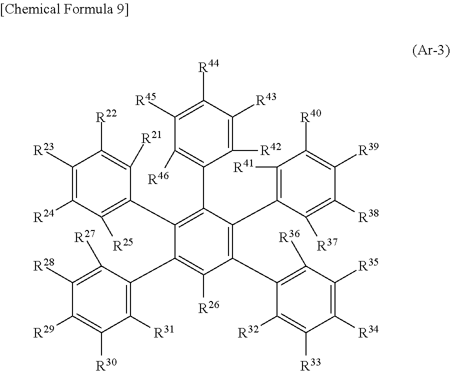

In General Formula (Ar-3), any m groups of R²¹ to R⁴⁶ represent m bonding positions in General Formula (G1), and each of the other groups of R²¹ to R⁴⁶ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

As described above, General Formula (Ar-3) shows the structure where Ar¹, Ar², and Ar³ in General Formula (Ar-2) are limited to substituted or unsubstituted benzene rings and p is limited to 3. When Ar¹, Ar², and Ar³ are limited to substituted or unsubstituted benzene rings, an increase in visible-light-absorption intensity of the organic compound of one embodiment of the present invention can be suppressed and a decrease in emission efficiency of the light-emitting device can be prevented. When p is 3, the periphery of the benzene ring (the ring A) at the center is congested as compared with the case where p is 2, so that the twist in the aromatic skeleton can be strengthened.

Alternatively, one embodiment of the present invention is an organic compound represented by General Formula (G2) below. Note that an organic compound represented by General Formula (G2) has a structure where the aromatic skeleton represented by General Formula (Ar-3) is used in the organic compound represented by General Formula (G1), k is limited to 1, and m is limited to 1. Thus, the description of the structure, effect, and the like of General Formulae (G1) and (Ar-1) to (Ar-3) can also be applied to General Formula (G2).

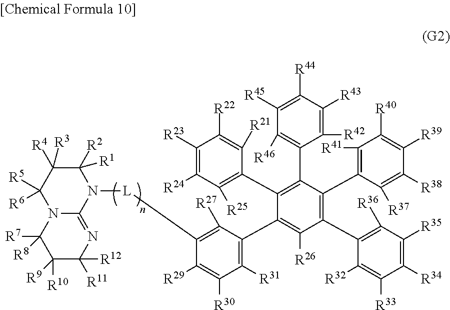

In General Formula (G2) above, L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; and each of R¹ to R¹² independently represents hydrogen (including deuterium) or an alkyl group having 1 to 10 carbon atoms. Note that when n is greater than or equal to 2, Ls may be the same or different from each other. Each of R²¹ to R²⁷ and R²⁹ to R⁴⁶ independently represents hydrogen (including deuterium), an alkyl group having 1 to 10 carbon atoms, a cycloalkyl group having 3 to 10 carbon atoms, a substituted or unsubstituted aryl group having 6 to 30 carbon atoms, or a substituted or unsubstituted heteroaryl group having 1 to 30 carbon atoms.

The organic compound represented by General Formula (G2) includes only one bicyclic guanidine skeleton in a molecule (corresponding to the case where m in General Formula (G1) is 1). Accordingly, the water solubility of the organic compound can be decreased as compared with the case where m is greater than or equal to 2.

The organic compound represented by General Formula (G2) includes the bicyclic guanidine skeleton at a position of R²⁸ in General Formula (Ar-3). This can reduce the water solubility and increase the heat resistance without hindering carrier transport.

Next, specific examples of an alkyl group having 1 to 10 carbon atoms, an alkylene group having 1 to 10 carbon atoms, a cycloalkyl group having 3 to 10 carbon atoms, an aryl group having 6 to 30 carbon atoms, an arylene group having 6 to 30 carbon atoms, and a heteroaryl group having 1 to 30 carbon atoms that can be used in General Formulae (G1), (Ar-1) to (Ar-3), and (G2) will be described. Note that in the specific examples described below, some or all of hydrogen atoms may be deuterium atoms. The groups that can be used in the above general formulae are not limited to the following specific examples.

### Specific Examples of Alkyl Group Having 1 to 10 Carbon Atoms

An alkyl group having 1 to 10 carbon atoms is a monovalent group with a structure where one hydrogen atom is removed from an alkane having 1 to 10 carbon atoms. Specific examples include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neo-pentyl group, a hexyl group, an isohexyl group, a sec-hexyl group, a tert-hexyl group, a neo-hexyl group, a 3-methylpentyl group, a 2-methylpentyl group, a 2-ethylbutyl group, a 1,2-dimethylbutyl group, a 2,3-dimethylbutyl group, and a 1-ethylhexyl group. In particular, the use of a branched alkyl group such as a tert-butyl group can further reduce the water solubility of the organic compound.

### Specific Examples of Alkylene Group Having 1 to 10 Carbon Atoms

An alkylene group having 1 to 10 carbon atoms is a bivalent group with a structure where two hydrogen atoms are removed from an alkane having Ito 19 carbon atoms. Specific examples include a bivalent group with a structure where one more hydrogen atom is removed from the above specific example of the alkyl group having 1 to 10 carbon atoms.

### Specific Examples of Cycloalkyl Group Having 3 to 10 Carbon Atoms

Specific examples of the cycloalkyl group having 3 to 10 carbon atoms include a cyclopropyl group, a cyclobutyl group, a methylcyclobutyl group, a cyclopentyl group, a methylcyclopentyl group, an isopropylcyclopentyl group, a tert-butylcyclopropyl group, a cyclohexyl group, a methylcyclohexyl group, an isopropylcyclohexyl group, a tert-butylcyclohexyl group, a cycloheptyl group, a methylcycloheptyl group, an isopropylcycloheptyl group, a cyclooctyl group, a methylcyclooctyl group, an isoproplylcyclohexyl group, a cyclononyl group, a methylcyclononyl group, a cyclodecyl group, and an adamantyl group.

### Specific Examples of Aryl Group Having 6 to 30 Carbon Atoms

An aryl group having 6 to 30 carbon atoms is a monovalent group with a structure where one hydrogen atom is removed from an aromatic hydrocarbon having 6 to 30 carbon atoms. Specific examples include a phenyl group, an o-tolyl group, an m-tolyl group, a p-tolyl group, a mesityl group, an o-biphenyl group, an m-biphenyl group, ap-biphenyl group, a 1-naphthyl group, a 2-naphthyl group, a fluorenyl group, a spirobifluorenyl group, a phenanthrenyl group, an anthracenyl group, and a fluoranthenyl group. In the case where the aryl group having 6 to 30 carbon atoms includes a substituent, examples of the substituent include an alkyl group having 1 to 10 carbon atoms, a cycloalkyl group having 3 to 10 carbon atoms, and a phenyl group.

### Specific Examples of Arylene Group Having 6 to 30 Carbon Atoms

An arylene group having 6 to 30 carbon atoms is a bivalent group with a structure where two hydrogen atoms are removed from an aromatic hydrocarbon having 6 to 30 carbon atoms. Specific examples include a bivalent group with a structure where one more hydrogen atom is removed from the above specific example of the aryl group having 6 to 30 carbon atoms. In the case where the arylene group having 6 to 30 carbon atoms includes a substituent, examples of the substituent include an alkyl group having 1 to 10 carbon atoms, a cycloalkyl group having 3 to 10 carbon atoms, and a phenyl group.

### Specific Examples of Heteroaryl Group Having 1 to 30 Carbon Atoms

Specific examples of the heteroaryl group having 1 to 30 carbon atoms include carbazole, benzocarbazole, dibenzocarbazole, indolocarbazole, benzindolocarbazole, dibenzindolocarbazole, benzindolobenzocarbazole, dibenzothiophene, benzonaphthothiophene, dibenzofuran, and benzonaphthofuran. In the case where the heteroaryl group having 1 to 30 carbon atoms includes a substituent, examples of the substituent include an alkyl group having 1 to 10 carbon atoms, a cycloalkyl group having 3 to 10 carbon atoms, and a phenyl group.

### Specific Examples of Heteroarylene Group Having 1 to 11 Carbon Atoms

Specific examples of a heteroarylene group having 1 to 11 carbon atoms include a pyridinediyl group, a pyrazinediyl group, a pyrimidinediyl group, a triazinediyl group, a triazolediyl group, an oxadiazolediyl group, a thiadiazolediyl group, an oxazolediyl group, a thiazolediyl group, a thiophenediyl group, a pyrrolediyl group, a furandiyl group, a selenophenediyl group, a benzothiophenediyl group, a benzopyrrolediyl group, a benzofurandiyl group, a quinolinediyl group, an isoquinolinediyl group, a dibenzothiophenediyl group, a carbazolediyl group, and a dibenzofurandiyl group. In the case where the heteroarylene group having 1 to 11 carbon atoms includes a substituent, examples of the substituent include an alkyl group having 1 to 10 carbon atoms, a cycloalkyl group having 3 to 10 carbon atoms, and a phenyl group.

The above examples of the substituent can be employed in the above general formulae.

Specific examples of the organic compound of one embodiment of the present invention represented by the above general formulae include organic compounds represented by Structural Formulae (100) to (146) below.

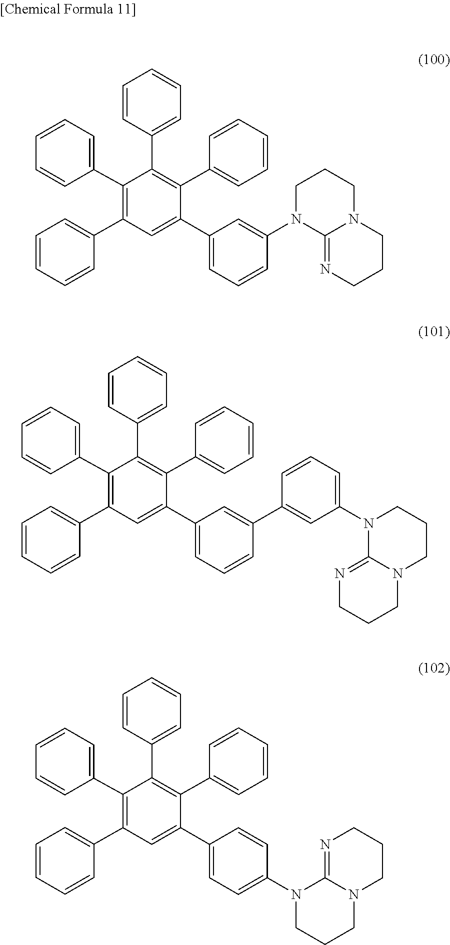

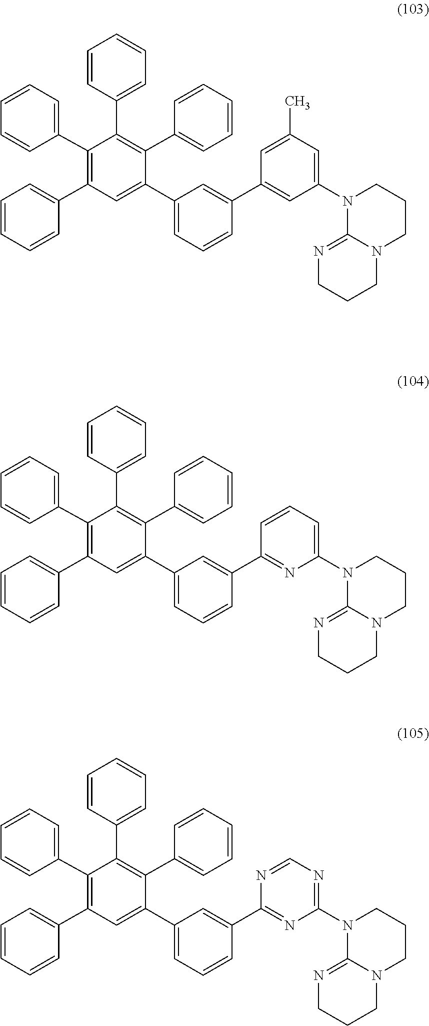

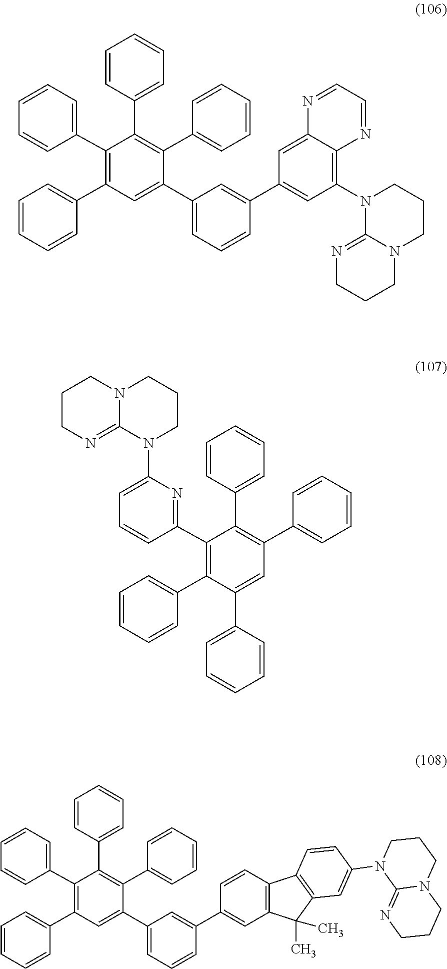

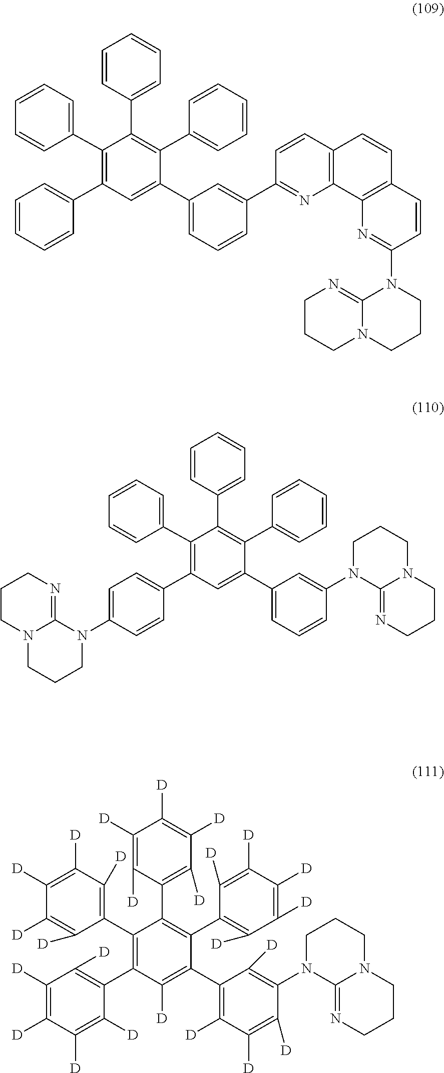

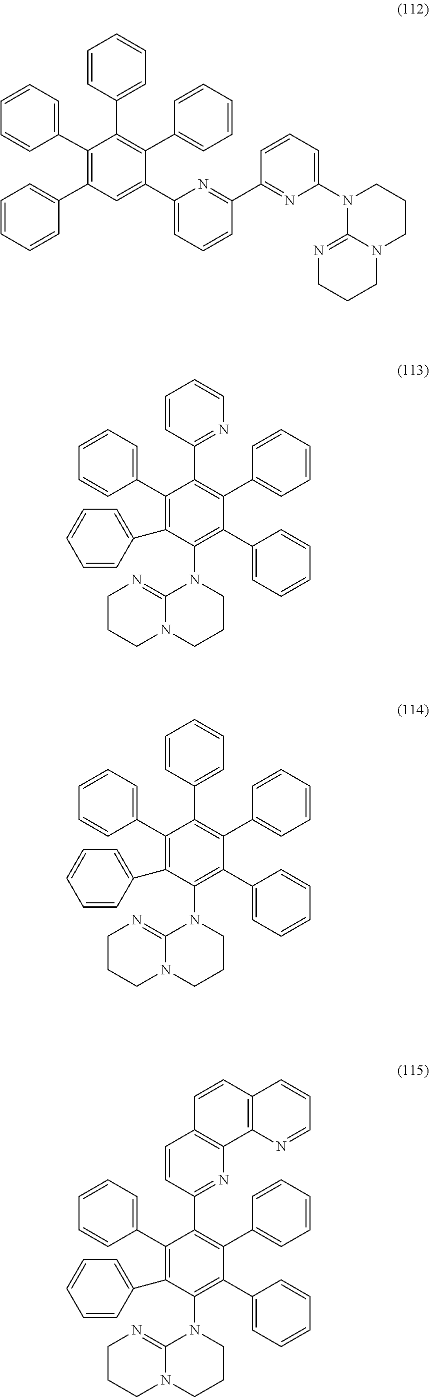

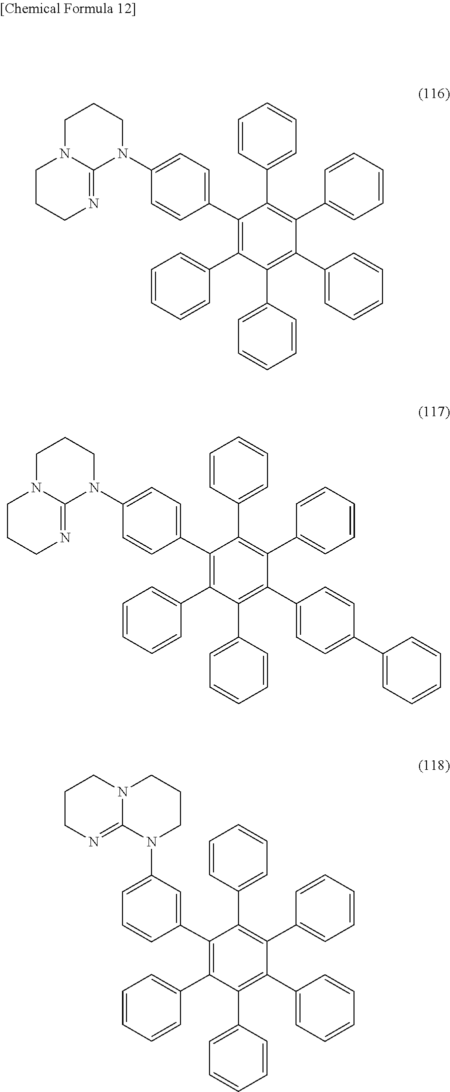

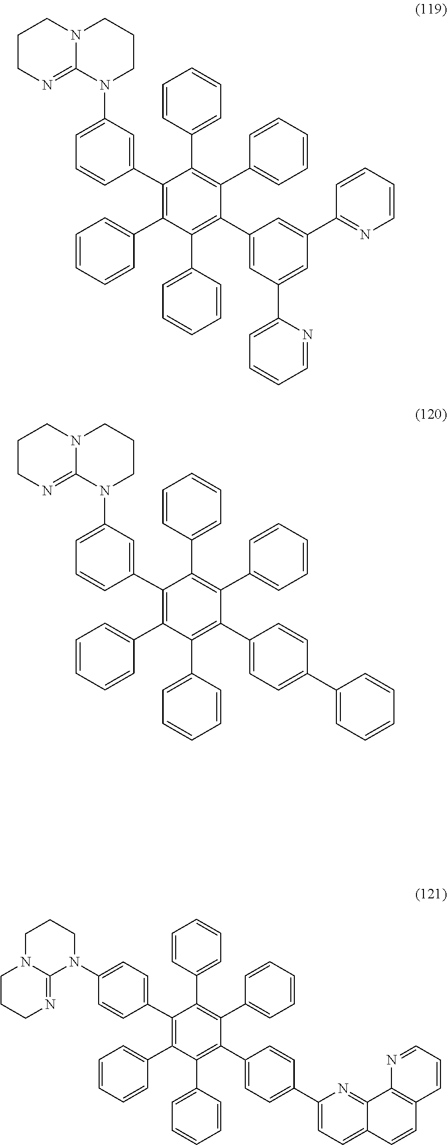

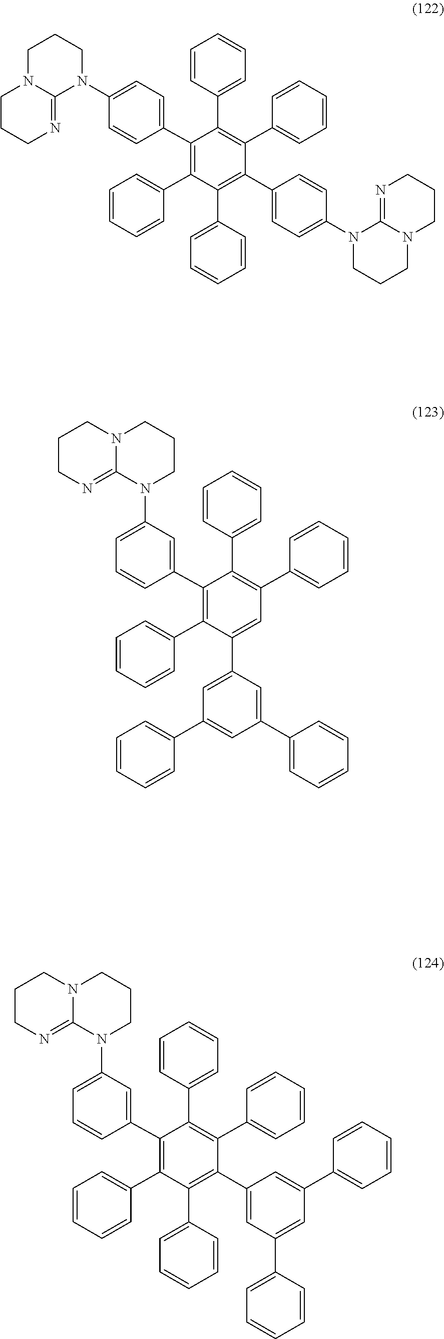

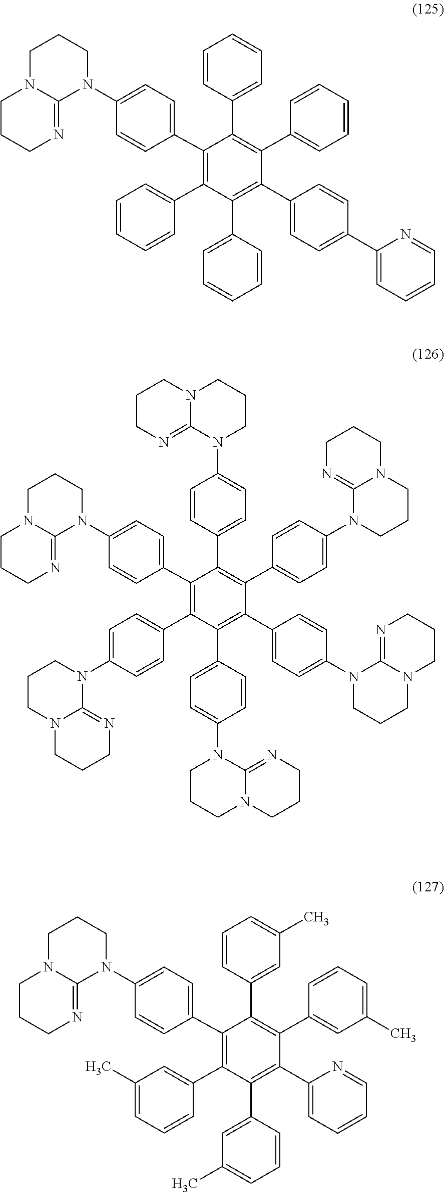

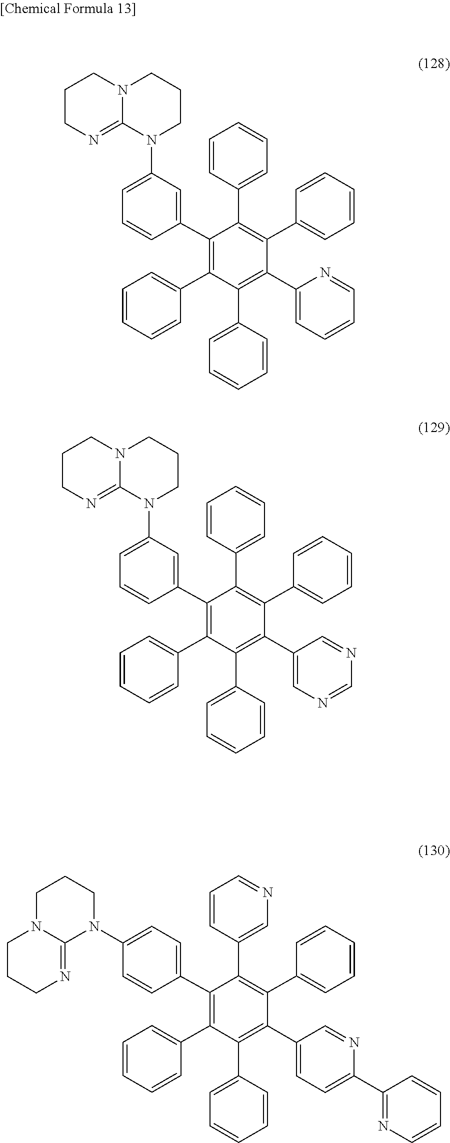

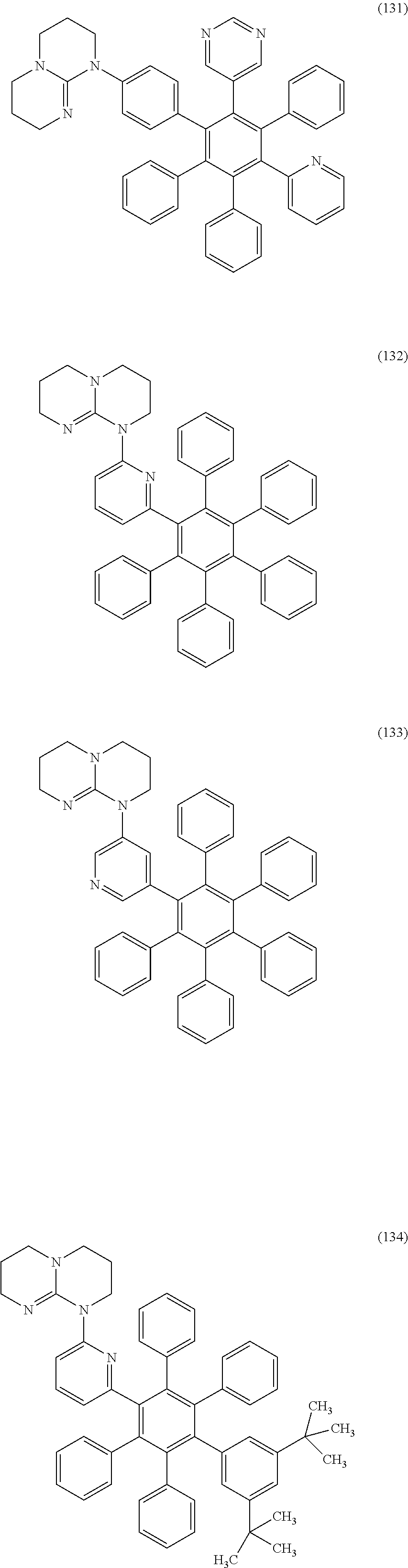

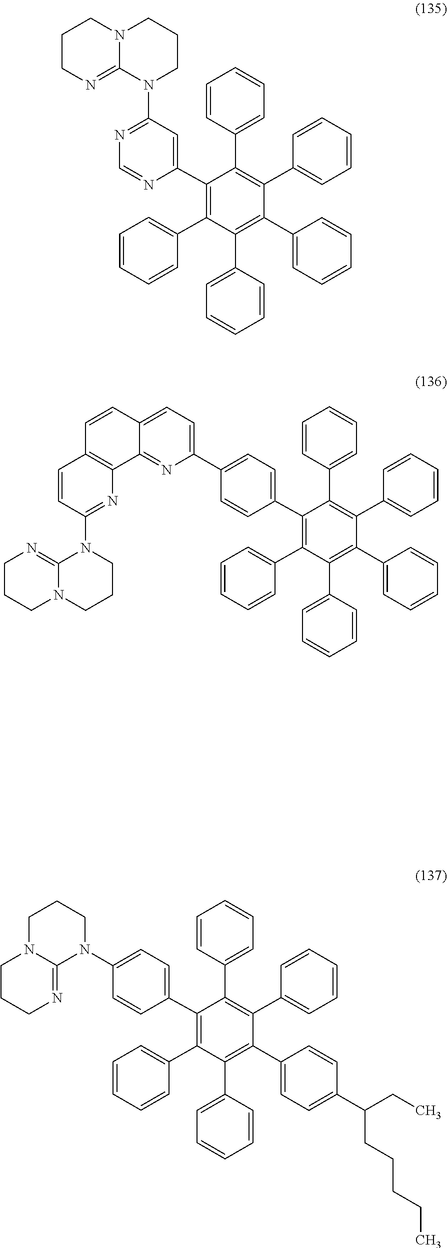

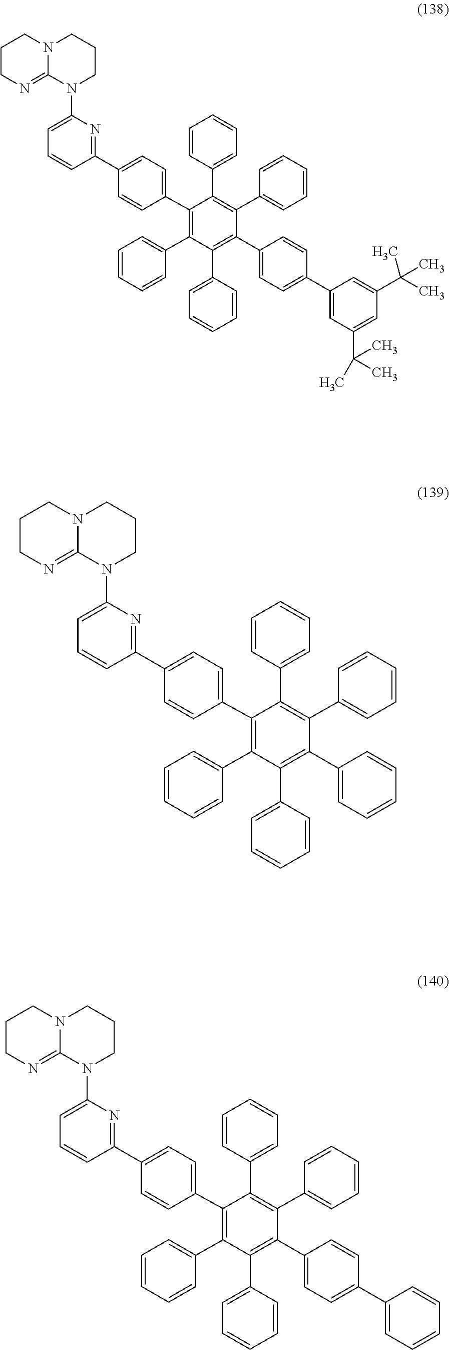

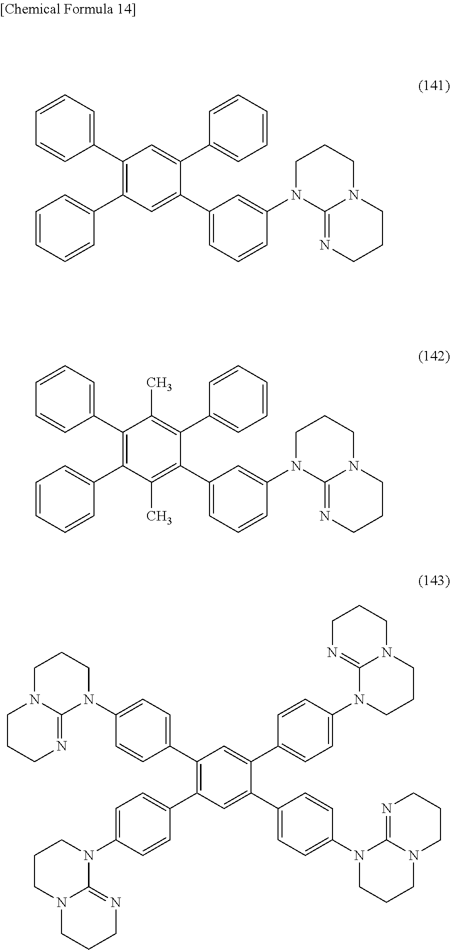

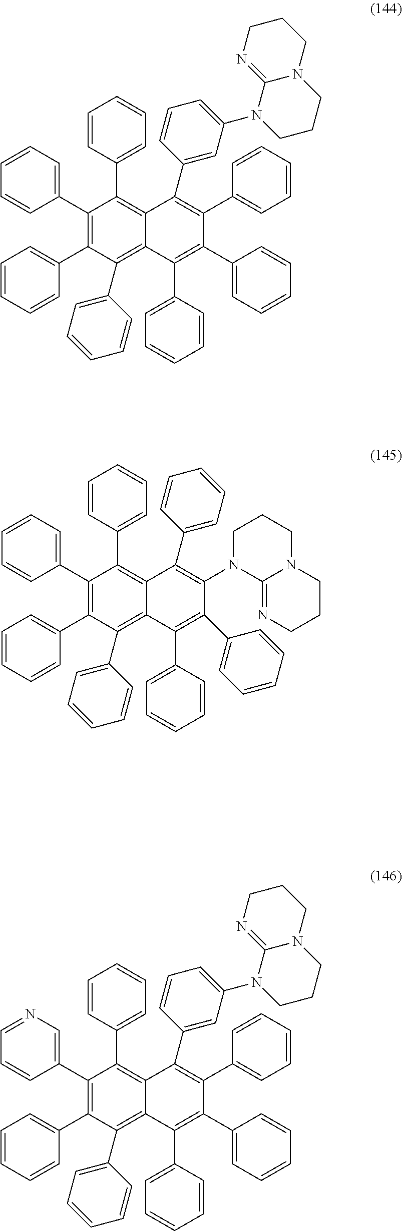

The organic compounds represented by Structural Formulae (100) to (146) above are examples of the organic compound of one embodiment of the present invention; however, one embodiment of the present invention is not limited thereto.

Next, a synthesis method of the organic compound represented by General Formula (G1) below which is an example of the organic compound of one embodiment of the present invention will be described. Note that the synthesis method of General Formula (G1) can employ a variety of reactions and is not limited to the following synthesis method.

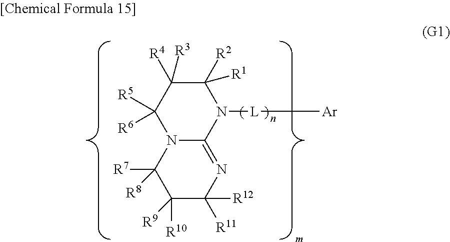

In General Formula (G1), Ar is an aromatic skeleton represented by any of General Formulae (Ar-1) to (Ar-3) described above; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; m represents an integer greater than or equal to 1 and less than or equal to 4; and each of R¹ to R¹² independently represents hydrogen (including deuterium) or an alkyl group having 1 to 10 carbon atoms. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other.

As shown in the following synthesis scheme, the organic compound represented by General Formula (G1) above can be obtained by coupling a halogen compound having an aromatic skeleton or a compound having an aromatic skeleton and a triflate group (a1) with a bicyclic guanidine derivative (a2) by Buchwald-Hartwig reaction.

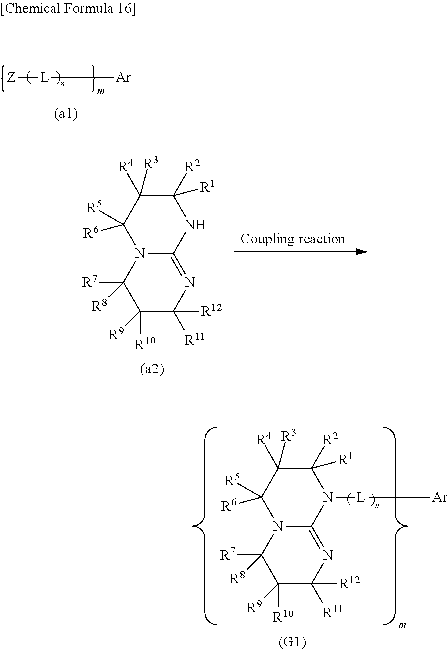

In General Formula (a1), Z represents halogen or a triflate group; Ar is an aromatic skeleton represented by any of General Formulae (Ar-1) to (Ar-3) described above; L represents an alkylene group having 1 to 10 carbon atoms, a substituted or unsubstituted arylene group having 6 to 30 carbon atoms, or a substituted or unsubstituted heteroarylene group having 1 to 11 carbon atoms; n represents an integer greater than or equal to 0 and less than or equal to 3; and m represents an integer greater than or equal to 1 and less than or equal to 4. In the case where n is greater than or equal to 2, Ls may be the same or different from each other. In the case where m is greater than or equal to 2, Ls may be the same or different from each other, n's may be the same or different from each other, and each of R¹s to R¹²s may be the same or different from each other.

In General Formula (a2), each of R¹ to R¹² independently represents hydrogen (including deuterium) or an alkyl group having 1 to 10 carbon atoms. In actual synthesis reaction, the organic compound represented by General Formula (a2) is preferably added excessively.

Examples of a palladium catalyst that can be used in the coupling reaction represented by the above synthesis scheme include palladium(II) acetate, tetrakis(triphenylphosphine)palladium(0), and bis(triphenylphosphine)palladium(II) dichloride.

Examples of a ligand in the above palladium catalyst include (±)-2,2′-bis(diphenylphosphino)-1,1′-binaphthyl, tri(ortho-tolyl)phosphine, triphenylphosphine, and tricyclohexylphosphine.

Examples of the base that can be used in the coupling reaction represented by the above synthesis scheme include an organic base such as potassium tert-butoxide and an inorganic base such as potassium carbonate or sodium carbonate.

Examples of a solvent that can be used in the coupling reaction represented by the above synthesis scheme include toluene, xylene, mesitylene, benzene, tetrahydrofuran, and dioxane. Note that the solvent that can be used is not limited thereto.

The reaction performed in Synthesis Schemes above is not limited to the Buchwald-Hartwig reaction. A Migita-Kosugi-Stille coupling reaction using an organotin compound, a coupling reaction using a Grignard reagent, an Ullmann reaction using copper or a copper compound, a nucleophilic substitution, or the like can be used.

A variety of kinds of the compound (a1) and the compound (a2) described above are commercially available or can be synthesized.

The organic compound of one embodiment of the present invention can be synthesized in the above manner, but the present invention is not limited to this and other synthesis methods may be employed for the synthesis.

Among the above organic compounds, an organic compound having low water solubility is preferable because of its high resistance to processing by a lithography method. For example, among the above organic compounds, an organic compound having water solubility lower than 3.9×10⁻⁴ g/mL at one atmospheric pressure and room temperature (RT) is preferable, and an organic compound having water solubility lower than 6.1×10⁻⁵ g/mL is further preferable. Note that the solubility in this specification refers to a value obtained by dividing the weight of a solute by the weight of a solution.

The structures described in this embodiment can be used in an appropriate combination with any of the structures described in the other embodiments.

### Embodiment 2

In this embodiment, structures of a light-emitting device using the organic compound of one embodiment of the present invention are described. Since the organic compound of one embodiment of the present invention is an organic compound having low water solubility, even when a light-emitting device using the organic compound of one embodiment of the present invention is manufactured by a manufacturing method including treatment using water, it is possible to prevent problems such as dissolution of a layer including the organic compound and permeation of a chemical solution into the layer including the organic compound; thus, the light-emitting device can have favorable characteristics.

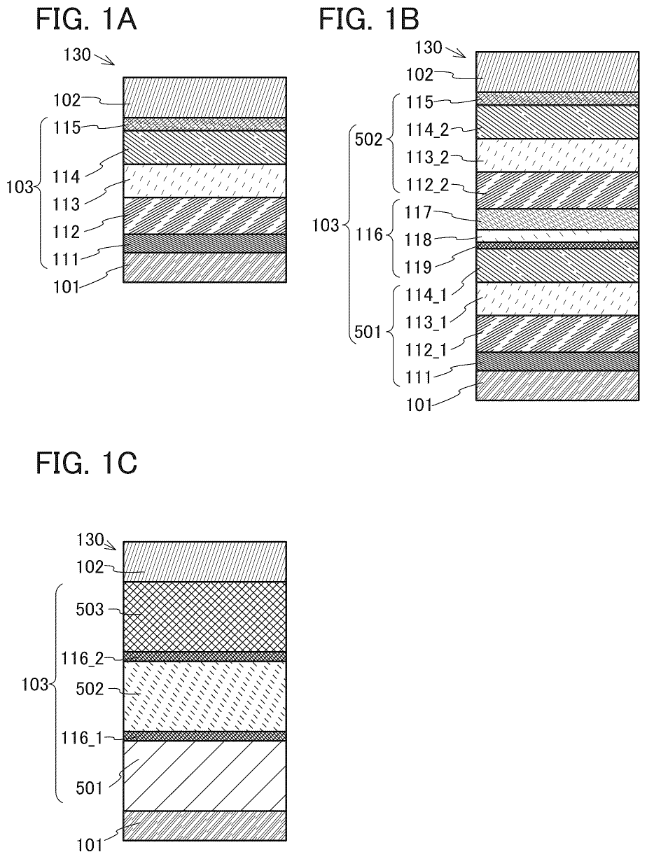

FIG. 1A illustrates a light-emitting device 130, which is an example of the light-emitting device of one embodiment of the present invention. The light-emitting device 130 is a light-emitting device that includes, between a first electrode 101 including an anode and a second electrode 102 including a cathode, an organic compound layer 103 including a light-emitting layer 113.

FIG. 1B illustrates the light-emitting device 130 that is another example of the light-emitting device of one embodiment of the present invention. The light-emitting device 130 is a tandem light-emitting device. The light-emitting device 130 includes, as the organic compound layer 103, a first light-emitting unit 501 including a first light-emitting layer 113_1, a second light-emitting unit 502 including a second light-emitting layer 1132, and an intermediate layer 116.

Although a light-emitting device including one intermediate layer 116 and two light-emitting units is described as an example in this embodiment, the light-emitting device may include n intermediate layers (n is an integer greater than or equal to 1) and n+1 light-emitting units.

For example, the light-emitting device 130 illustrated in FIG. 1C is an example of a tandem light-emitting device with n=2 that includes, as the organic compound layer 103, the first light-emitting unit 501, a first intermediate layer 116_1, the second light-emitting unit 502, a second intermediate layer 1162, and a third light-emitting unit 503. The color gamut of light emitted by the light-emitting layers in the light-emitting units may be the same or different. In addition, the light-emitting layer may have a single-layer structure or a stacked-layer structure. For example, white light emission can be obtained with a structure where light-emitting layers in the first light-emitting unit and the third light-emitting unit emit light in a blue region and a light-emitting layer having a stacked-layer structure in the second light-emitting unit emit light in a red region and light in a green region.

The light-emitting device 130 may be a light-emitting device manufactured by a lithography method, for example. In the case of the light-emitting device manufactured by a lithography method, at least the light-emitting layer 113 or the second light-emitting layer 113_2 and the organic compound layers which are closer to the first electrode 101 than the light-emitting layer 113 or the second light-emitting layer 113_2 are processed at the same time; thus, the end portions of the layers are substantially aligned in the perpendicular direction.

The organic compound layer 103 may include another functional layer in addition to the light-emitting layer. FIG. 1A illustrates a structure where, in addition to the light-emitting layer 113, a hole-injection layer 111, a hole-transport layer 112, an electron-transport layer 114, and an electron-injection layer 115 are provided in the organic compound layer 103. The first light-emitting unit 501 and the second light-emitting unit 502 may each include another functional layer in addition to the light-emitting layer. FIG. 1B illustrates a structure where the first light-emitting unit 501 is provided with the hole-injection layer 111, a first hole-transport layer 1121, and a first electron-transport layer 114_1 in addition to the first light-emitting layer 113_1 and the second light-emitting unit 502 is provided with a second hole-transport layer 1122, a second electron-transport layer 1142, and the electron-injection layer 115 in addition to the second light-emitting layer 113_2. However, the structure of the organic compound layer 103 of the present invention is not limited to this structure; any of the layers may be absent or another layer may be added. Typical example of the another layer include a carrier-blocking layer (a hole-blocking layer or an electron-blocking layer) and an exciton-blocking layer.

### <<Structure of Intermediate Layer>>

First, a material that can be used for the intermediate layer 116 is described. The organic compound of one embodiment of the present invention described in Embodiment 1 can be used for the intermediate layer 116. The intermediate layer 116 preferably has a stacked-layer structure including a first layer 119 and a second layer 117, and instead of the donor substance, the organic compound of one embodiment of the present invention described in Embodiment 1 is preferably used for the first layer 119. Since the organic compound of one embodiment of the present invention is an organic compound having low water solubility, even when a light-emitting device using the organic compound for an intermediate layer is manufactured by a manufacturing method including treatment using water, it is possible to prevent problems such as dissolution of a layer including the organic compound and permeation of a chemical solution into the layer including the organic compound; thus, the light-emitting device can have favorable characteristics.

The second layer 117 is positioned closer to the second electrode 102 than the first layer 119 is. Between the first layer 119 and the second layer 117, a third layer 118 for smooth donation and acceptance of electrons between the two layers may be provided.

Since the first layer 119 is included in the intermediate layer 116, the first layer 119 serves as an electron-injection layer in the light-emitting unit on the anode side. Thus, an electron-injection layer may be but is not necessarily included in the light-emitting unit on the anode side (the first light-emitting unit 501 in FIG. 1). Similarly, since the second layer 117 is included in the intermediate layer 116, the second layer 117 serves as a hole-injection layer in the light-emitting unit on the cathode side. Thus, a hole-injection layer may be but is not necessarily included in the light-emitting unit on the cathode side (the second light-emitting unit 502 in FIG. 1B).

Note that the first layer 119 may include an organic compound having an electron-transport property in addition to the organic compound of one embodiment of the present invention. For example, as the first layer 119, a mixed layer of the organic compound of one embodiment of the present invention and an organic compound having an electron-transport property can be used. When the organic compound of one embodiment of the present invention and an organic compound having an electron-transport property are used for the first layer 119, the light-emitting device can have higher emission efficiency.

The organic compound having an electron-transport property that can be used for the first layer 119 is preferably a substance having an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property.

An organic compound having a π-electron deficient heteroaromatic ring is preferable as the above organic compound. The organic compound having a π-electron deficient heteroaromatic ring is preferably one or more of an organic compound having a heteroaromatic ring having a polyazole skeleton, an organic compound having a heteroaromatic ring having a pyridine skeleton, an organic compound having a heteroaromatic ring having a diazine skeleton, and an organic compound having a heteroaromatic ring having a triazine skeleton.

Specific examples of the organic compound having an electron-transport property that can be used for the first layer 119 include organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: COl1), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); organic compounds having a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: Bphen), bathocuproine (abbreviation: BCP), 2,9-di(naphthalen-2-yl)-4,7-diphenyl-1,10-phenanthroline (abbreviation: NBphen), and 2,2′-(1,3-phenylene)bis(9-phenyl-1,10-phenanthroline) (abbreviation: mPPhen2P); organic compounds having a diazine skeleton, such as 2-[3-(dibenzothiophen-4-yl)phenyl]dibenzo[f,h]quinoxaline (abbreviation: 2mDBTPDBq-II), 2-[3-(3′-dibenzothiophen-4-yl)biphenyl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′: 4,5]furo[2,3-b]pyrazine (abbreviation: 9mDBtBPNfpr), 9-[(3′-dibenzothiophen-4-yl)biphenyl-4-yl]naphtho[1′,2′: 4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′: 4,5]furo[3,2-d]pyrimidine (abbreviation: 8mDBtBPNfpm), 8-[(2,2′-binaphthalen)-6-yl]-4-[3-(dibenzothiophen-4-yl)phenyl]-[1]benzofuro[3,2-d]pyrimidine (abbreviation: 8(PN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), and 7-[4-(9-phenyl-9H-carbazol-2-yl)quinazolin-2-yl]-7H-dibenzo[c,g]carbazole (abbreviation: PC-cgDBCzQz); and organic compounds having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris(3′-(pyridin-3-yl)biphenyl-3-yl)-1,3,5-triazine (abbreviation: TmPPPyTz), 2-[3-(2,6-dimethyl-3-pyridinyl)-5-(9-phenanthrenyl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), and 2-(biphenyl-3-yl)-4-phenyl-6-{8-[(1,1′:4′,1″-terphenyl)-4-yl]-1-dibenzofuranyl}-1,3,5-triazine (abbreviation: mBP-TPDBfTzn). In particular, organic compounds having a phenanthroline skeleton, such as Bphen, BCP, NBphen, and mPPhen2P, are preferable, and an organic compound having a phenanthroline dimeric structure, such as mPPhen2P, is further preferable because of its excellent stability.

The second layer 117 is preferably formed using a composite material including the above material having an acceptor property and an organic compound having a hole-transport property. As the organic compound having a hole-transport property used for the composite material, any of a variety of organic compounds such as aromatic amine compounds, heteroaromatic compounds, aromatic hydrocarbons, and high molecular compounds (e.g., oligomers, dendrimers, or polymers) can be used. Note that the organic compound having a hole-transport property used for the composite material preferably has a hole mobility higher than or equal to 1×10⁻⁶ cm²/Vs. The organic compound having a hole-transport property used for the composite material is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton in the ring is preferable; specifically, a carbazole ring, a dibenzothiophene ring, or a ring in which an aromatic ring or a heteroaromatic ring is further condensed to the carbazole ring or the dibenzothiophene ring is preferable.

Such an organic compound having a hole-transport property further preferably has any of a carbazole skeleton, a dibenzofuran skeleton, a dibenzothiophene skeleton, and an anthracene skeleton. In particular, an aromatic amine including a substituent having a dibenzofuran ring or a dibenzothiophene ring, an aromatic monoamine having a naphthalene ring, or an aromatic monoamine in which a 9-fluorenyl group is bonded to nitrogen of amine through an arylene group may be used. Note that the organic compound having a hole-transport property preferably has an N,N-bis(4-biphenyl)amino group because a light-emitting device having a long lifetime can be manufactured.

Specific examples of the above-described organic compounds having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNPNB), 4,4′-diphenyl-4″-(7;1′-binaphthyl-2-yl)triphenylamine (abbreviation: BBAαNPNB-03), 4,4′-diphenyl-4″-(7-phenyl)naphthyl-2-yltriphenylamine (abbreviation: BBAβPNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiPNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

As the material having a hole-transport property, any of the following aromatic amine compounds can also be used: N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N′-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B), and the like.

Examples of the substance having an acceptor property that is included in the second layer 117 include organic compounds having an electron-withdrawing group (a halogen group, a cyano group, or the like), such as 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F₆-TCNNQ), and 2-(7-dicyanomethylen-1,3,4,5,6,8,9,10-octafluoro-7H-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as a molybdenum oxide, a vanadium oxide, a ruthenium oxide, a tungsten oxide, or a manganese oxide can also be used, other than the above-described organic compounds.

The third layer 118 contains a substance having an electron-transport property and has a function of preventing an interaction between the first layer 119 and the second layer 117 and smoothly transferring electrons. The LUMO level of the substance having an electron-transport property contained in the third layer 118 is preferably between the LUMO level of the acceptor substance in the second layer 117 and the LUMO level of an organic compound contained in a layer that is included in the light-emitting unit on the first electrode 101 side and is in contact with the intermediate layer 116 (the first electron-transport layer 1141 in the first light-emitting unit 501 in FIG. 1i). As a specific value of the energy level, the LUMO level of the substance having an electron-transport property used for the third layer 118 is preferably higher than or equal to −5.0 eV, further preferably higher than or equal to −5.0 eV and lower than or equal to −3.0 eV, still further preferably higher than or equal to −4.30 eV and lower than or equal to −3.00 eV, yet still further preferably higher than or equal to −4.30 eV and lower than or equal to −3.30 eV, in which case electrons generated in the second layer 117 can be easily injected to the first layer 119 and accordingly an increase in driving voltage of the light-emitting device can be suppressed. Note that as the substance having an electron-transport property used for the third layer 118, a phthalocyanine-based material or a metal complex having a metal-oxygen bond and an aromatic ligand is preferably used.

Specifically, it is possible to use a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI), (C60-Iₕ)[5,6]fullerene (abbreviation: C₆₀), (C70-D5h)[5,6]fullerene (abbreviation: C₇₀), or phthalocyanine (abbreviation: H₂Pc). Alternatively, it is possible to use a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). It is particularly preferable to use a phthalocyanine-based metal complex such as copper phthalocyanine or zinc phthalocyanine or 2,3,8,9,14,15-hexafluorodiquinoxalino[2,3-a:2′,3′-c]phenazine.

The thickness of the third layer 118 is preferably greater than or equal to 1 nm and less than or equal to 10 nm, further preferably greater than or equal to 2 nm and less than or equal to 5 nm.

Then, components of the above light-emitting device 130, other than the intermediate layer 116, are described.

### <<Structure of First Electrode>>

The first electrode 101 is an electrode including an anode. The first electrode 101 may have a stacked-layer structure where a layer in contact with the organic compound layer 103 functions as the anode. The anode is preferably formed using any of a metal, an alloy, and a conductive compound with a high work function (specifically, higher than or equal to 4.0 eV), a mixture thereof, or the like. Specific examples include indium oxide-tin oxide (ITO: Indium Tin Oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, indium oxide-zinc oxide is formed by a sputtering method using a target obtained by adding 1 to 20 wt % of zinc oxide to indium oxide. Furthermore, indium oxide containing tungsten oxide and zinc oxide (IWZO) can be formed by a sputtering method using a target in which tungsten oxide and zinc oxide are added to indium oxide at 0.5 to 5 wt % and 0.1 to 1 wt %, respectively. Other examples of the material used for the anode include gold (Au), platinum (Pt), nickel (Ni), tungsten (W), chromium (Cr), molybdenum (Mo), iron (Fe), cobalt (Co), copper (Cu), palladium (Pd), and a nitride of a metal material (e.g., titanium nitride). Graphene can also be used for the anode. Note that when the composite material contained in the second layer 117 in the intermediate layer 116 is used for a layer (typically, a hole-injection layer) that is in contact with the anode, an electrode material can be selected regardless of its work function.

### <<Structure of Hole-Injection Layer>>

The hole-injection layer 111 is provided in contact with the anode and has a function of facilitating injection of holes into the organic compound layer 103 (the first light-emitting unit 501). The hole-injection layer 111 can be formed using phthalocyanine (abbreviation: H₂Pc), a phthalocyanine-based compound or complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N-(3-methylphenyl)-N-phenylamino]phenyl}-N-phenylamino)biphenyl (abbreviation: DNTPD), a high molecular compound such as poly(3,4-ethylenedioxythiophene)/(polystyrenesulfonic acid) (abbreviation: PEDOT/PSS), or the like.

The hole-injection layer 111 may be formed using a substance having an electron-acceptor property. As the substance having an acceptor property, any of substances described as examples of the acceptor substance used for the composite material contained in the second layer 117 in the intermediate layer 116 can similarly be used.

The composite material contained in the second layer 117 in the intermediate layer 116 may be similarly used to form the hole-injection layer 111.

Further preferably, in the hole-injection layer 111, the organic compound having a hole-transport property used for the composite material has a relatively deep HOMO level higher than or equal to −5.7 eV and lower than or equal to −5.4 eV. When the organic compound having a hole-transport property used for the composite material has a relatively deep HOMO level, holes can be easily injected into the hole-transport layer to easily provide a light-emitting device having a long lifetime. In addition, when the organic compound having a hole-transport property used for the composite material has a relatively deep HOMO level, induction of holes can be inhibited properly so that a light-emitting device having a longer lifetime can be obtained.

The formation of the hole-injection layer 111 can improve the hole-injection property, whereby a light-emitting device having a low driving voltage can be obtained.

Among substances having an acceptor property, an organic compound having an acceptor property is easy to use because it is easily deposited by vapor deposition.

Since the second layer 117 in the intermediate layer 116 functions as a hole-injection layer, another hole-injection layer is not provided in the second light-emitting unit 502; however, a hole-injection layer may be provided in the second light-emitting unit 502.

The hole-transport layer (the first hole-transport layer 112_1 or the second hole-transport layer 112_2) includes an organic compound having a hole-transport property. The organic compound having a hole-transport property preferably has a hole mobility higher than or equal to 1×10⁻⁶ cm²/Vs.

Examples of the material having a hole-transport property include compounds having an aromatic amine skeleton, such as 4,4′-bis[N-(1-naphthyl)-N-phenylamino]biphenyl (abbreviation: NPB), NN-diphenyl-N,N′-bis(3-methylphenyl)-4,4′-diaminobiphenyl (abbreviation: TPD), N,N′-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bicarbazole (abbreviation: βNCCP), 9-(3-biphenyl)-9′-(2-naphthyl)-3,3′-bi-9H-carbazole (abbreviation: PNCCmBP), 9-(4-biphenyl)-9′-(2-naphthyl)-3,3′-bi-9H-carbazole (abbreviation: PNCCBP), 9,9′-di-2-naphthyl-3,3′-9H,9′H-bicarbazole (abbreviation: BisPNCz), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′:3′,1″-terphenyl]-5′-yl-3,3′-9H,9′H-bicarbazole, 9-(2-naphthyl)-9′-[1,1′:4′,1″-terphenyl]-4-yl-3,3′-9H,9′H-bicarbazole, 9-(2-naphthyl)-9′-[1,1′:3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, and 9-(triphenylen-2-yl)-9′-[1,1′:3′,1″-terphenyl]-4-yl-3,3′-9H,9′H-bicarbazole; compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. Note that any of the substances given as examples of the material having a hole-transport property used for the composite material for the hole-injection layer 111 can also be suitably used as the material contained in the hole-transport layer.

### <<Structure of Light-Emitting Layer>>

The light-emitting layer (the light-emitting layer 113, the first light-emitting layer 113_1 and the second light-emitting layer 1132) preferably contains a light-emitting substance and a host material. The light-emitting layer may additionally contain other materials. Alternatively, the light-emitting layer may be a stack of two layers with different compositions.

The light-emitting substance may be a fluorescent substance, a phosphorescent substance, a substance exhibiting thermally activated delayed fluorescence (TADF), or any of other light-emitting substances.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,NA-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis[N,N′,N′-triphenyl-1,4-phenylenediamine](abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), 9,10-bis(2-biphenyl)-2-(N,N′,N′-triphenyl-1,4-phenylenediamine-N-yl)anthracene (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N-diphenyl-N,N-(1,6-pyrene-diyl)bis[(6-phenylbenzo[b]naphtho[1,2-d]furan)-8-amine](abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, or high reliability.

Examples of the material that can be used when a phosphorescent substance is used as the light-emitting substance in the light-emitting layer are as follows.

The examples include organometallic iridium complexes having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); organometallic iridium complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having an imidazole skeleton, such asfac-tris[1-(2,6-diisopropylphenyl)-2-phenyl-1H-imidazole]iridium(III) (abbreviation: [Ir(iPrpim)₃]) and tris[3-(2,6-dimethylphenyl)-7-methylimidazo[1,2-f]phenanthridinato]iridium(III) (abbreviation: [Ir(dmpimpt-Me)₃]); and organometallic iridium complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac). These compounds exhibit blue phosphorescent light and have an emission peak in the wavelength range of 450 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN2)phenyl-κC]iridium(III) (abbreviation: Ir(5mppy-d₃)₂(mbfpypy-d₃)), {2-(methyl-d₃)-8-[4-(1-methylethyl-1-d)-2-pyridinyl-KN]benzofuro[2,3-b]pyridin-7-yl-κC}bis{5-(methyl-d₃)-2-[5-(methyl-d₃)-2-pyridinyl-KN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); and a rare earth metal complex such as tris(acetylacetonato)(monophenanthroline)terbium(III) (abbreviation: [Tb(acac)₃(Phen)]). These are mainly compounds that exhibit green phosphorescent light and have an emission peak in the wavelength range of 500 nm to 600 nm. Note that organometallic iridium complexes having a pyrimidine skeleton have distinctively high reliability or emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]) and bis(1-phenylisoquinolinato-N,C^{2′})iridium(III) acetylacetonate (abbreviation: [Ir(piq)₂(acac)]); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds exhibit red phosphorescent light and have an emission peak in the wavelength range of 600 nm to 700 nm. Organometallic iridium complexes having a pyrazine skeleton can provide red light emission with favorable chromaticity.

Besides the above phosphorescent compounds, known phosphorescent compounds may be selected and used.

As the TADF material, a fullerene, a derivative thereof, an acridine, a derivative thereof, an eosin derivative, or the like can be used. Furthermore, a metal-containing porphyrin, such as a porphyrin containing magnesium (Mg), zinc (Zn), cadmium (Cd), tin (Sn), platinum (Pt), indium (In), or palladium (Pd), can be given as an example. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

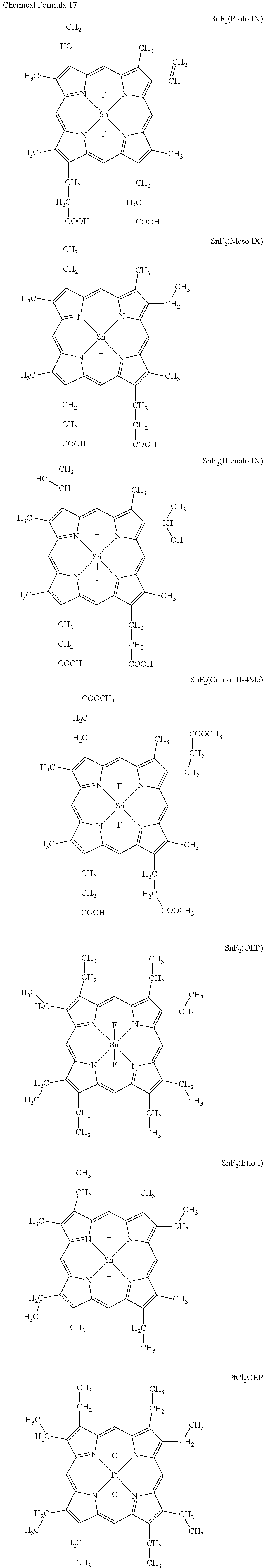

A heterocyclic compound having one or both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. Such a heterocyclic compound is preferable because of having excellent electron-transport and hole-transport properties owing to a π-electron rich heteroaromatic ring and a π-electron deficient heteroaromatic ring. Among skeletons having a π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. Note that a dibenzofuran skeleton and a dibenzothiophene skeleton are preferable as a furan skeleton and a thiophene skeleton, respectively. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring is directly bonded to a π-electron deficient heteroaromatic ring is particularly preferable because the electron-donating property of the π-electron rich heteroaromatic ring and the electron-accepting property of the π-electron deficient heteroaromatic ring are both improved, the energy difference between the S1 level and the T1 level becomes small, and thus thermally activated delayed fluorescence can be obtained efficiently. Note that an aromatic ring to which an electron-withdrawing group such as a cyano group is bonded may be used instead of a π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring having a cyano group or a nitrile group such as benzonitrile or cyanobenzene, a heteroaromatic ring, a carbonyl skeleton such as benzophenone, a phosphine oxide skeleton, a sulfone skeleton, or the like can be used. As described above, a π-electron deficient skeleton and a π-electron rich skeleton can be used instead of at least one of a π-electron deficient heteroaromatic ring and a π-electron rich heteroaromatic ring.

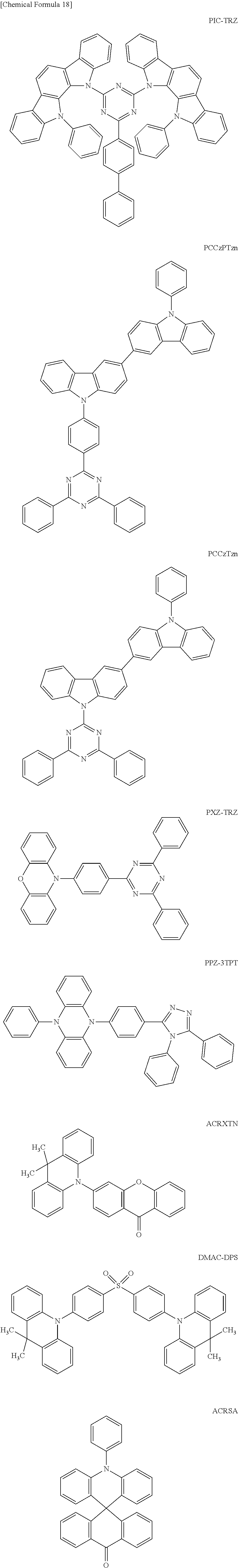

As the TADF material, a TADF material whose singlet excited state and triplet excited state are in a thermal equilibrium state may be used. Since such a TADF material enables a short emission lifetime (excitation lifetime), a decrease in efficiency of a light-emitting device in a high-luminance region can be inhibited. Specifically, a material having the following molecular structure can be used.

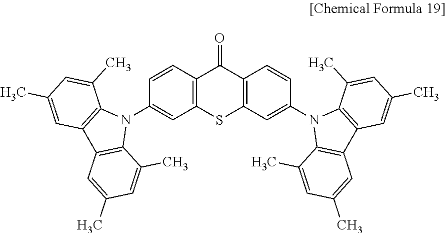

Note that a TADF material is a material having a small difference between the S1 level and the T1 level and a function of converting triplet excitation energy into singlet excitation energy by reverse intersystem crossing. Thus, it is possible to upconvert triplet excitation energy into singlet excitation energy (i.e., reverse intersystem crossing) using a small amount of thermal energy and efficiently generate a singlet excited state. In addition, the triplet excitation energy can be converted into luminescence.

An exciplex whose excited state is formed of two kinds of substances has an extremely small difference between the S1 level and the T1 level and has a function of a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the S1 level and the level of energy with a wavelength of the line obtained by extrapolating a tangent to the phosphorescent spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than the S1 level of the TADF material. In addition, the T1 level of the host material is preferably higher than the T1 level of the TADF material.

As the host material in the light-emitting layer, various carrier-transport materials such as materials having an electron-transport property and/or materials having a hole-transport property, and the TADF materials can be used.

As the material having a hole-transport property, the above materials given as the material having a hole-transport property can be similarly used.

As the material having an electron-transport property, the above materials given as the material having an electron-transport property can be similarly used.

As the TADF material that can be used as the host material, the above materials given as the TADF material can be similarly used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S1 level of the TADF material is preferably higher than the S1 level of the fluorescent substance in order that high emission efficiency be achieved. Furthermore, the T1 level of the TADF material is preferably higher than the S1 level of the fluorescent substance. Thus, the T1 level of the TADF material is preferably higher than the T1 level of the fluorescent substance.

It is also preferable to use a TADF material that emits light whose wavelength overlaps with the wavelength of a lowest-energy-side absorption band of the fluorescent substance. This enables smooth transfer of excitation energy from the TADF material to the fluorescent substance and accordingly enables efficient light emission, which is preferable.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protecting group around a luminophore (a skeleton which causes light emission) of the fluorescent substance. As the protecting group, a substituent having no π bond is preferable, and a saturated hydrocarbon is preferable. Specific examples include an alkyl group having 3 to 10 carbon atoms, a substituted or unsubstituted cycloalkyl group having 3 to 10 carbon atoms, and a trialkylsilyl group having 3 to 10 carbon atoms. The substituent having no π bond is poor in carrier-transport performance; thus, the TADF material and the luminophore of the fluorescent substance can be made away from each other with little influence on carrier-transportation or carrier recombination. Here, the luminophore refers to an atomic group (skeleton) that causes light emission in a fluorescent substance. The luminophore is preferably a skeleton having a π bond, further preferably includes an aromatic ring, and still further preferably includes a condensed aromatic ring or a condensed heteroaromatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a material having an anthracene skeleton is suitable as the host material. The use of a substance having an anthracene skeleton as the host material for the fluorescent substance makes it possible to obtain a light-emitting layer having both high emission efficiency and high durability. As the substance having an anthracene skeleton that is used as the host material, a substance having a diphenylanthracene skeleton, in particular, a substance having a 9,10-diphenylanthracene skeleton, is chemically stable and thus is preferable. The host material preferably has a carbazole skeleton, in which case the hole-injection and hole-transport properties are improved; further preferably, the host material has a benzocarbazole skeleton in which a benzene ring is further condensed to a carbazole skeleton because the HOMO level thereof is shallower than that of the host material having a carbazole skeleton by approximately 0.1 eV, so that holes enter the host material easily. In particular, the host material preferably has a dibenzocarbazole skeleton, in which case the HOMO level thereof is shallower than that of the host material having a carbazole skeleton by approximately 0.1 eV, so that holes enter the host material easily, the hole-transport property is improved, and the heat resistance is increased. Accordingly, a substance having both a 9,10-diphenylanthracene skeleton and a carbazole skeleton (or a benzocarbazole skeleton or a dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9-(1-naphthyl)-10-[4-(2-naphthyl)phenyl]anthracene (abbreviation: α,N-βNPAnth), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthracenyl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

The host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, the phosphorescent substance can be used as an energy donor for supplying excitation energy to the fluorescent substance.

An exciplex may be formed of these mixed materials. These mixed materials are preferably selected to form an exciplex that exhibits light emission overlapping with the wavelength of a lowest-energy-side absorption band of the light-emitting substance, in which case energy can be transferred smoothly and light emission can be obtained efficiently. The use of such a structure is preferable because the driving voltage can also be reduced.

At least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to the LUMO level of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed, for example, in the following manner: when the emission spectrum of the material having a hole-transport property, the emission spectrum of the material having an electron-transport property, and the emission spectrum of a mixed film of these materials are compared, a phenomenon in which the emission spectrum of the mixed film is shifted to the longer wavelength than the emission spectrum of each of the materials (or has another peak on the longer wavelength side) is observed. Alternatively, when the transient photoluminescence (PL) of the material having a hole-transport property, the transient PL of the material having an electron-transport property, and the transient PL of the mixed film of these materials are compared, a difference in transient response is observed, for example, the transient PL lifetime of the mixed film has a longer lifetime component or has a larger portion of a delayed component than that of each of the materials. The transient PL can be rephrased as transient electroluminescence (EL). That is, the formation of an exciplex can also be confirmed by comparing the transient EL of the material having a hole-transport property, the transient EL of the material having an electron-transport property, and the transient EL of the mixed film of these materials and observing a difference in transient response.

### <<Structure of Electron-Transport Layer>>

The electron-transport layer (the electron-transport layer 114, the first electron-transport layer 114_1, and the second electron-transport layer 1142) is a layer containing a substance having an electron-transport property. The material having an electron-transport property is preferably a substance having an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property. An organic compound having a π-electron deficient heteroaromatic ring is preferable as the above organic compound. The organic compound having a π-electron deficient heteroaromatic ring is preferably one or more of an organic compound having a heteroaromatic ring having a polyazole skeleton, an organic compound having a heteroaromatic ring having a pyridine skeleton, an organic compound having a heteroaromatic ring having a diazine skeleton, and an organic compound having a heteroaromatic ring having a triazine skeleton.

As the organic compound having an electron-transport property that can be used for the electron-transport layer, the organic compound that can be used as the organic compound having an electron-transport property in the first layer of the intermediate layer 116 can be similarly used. Among them, the organic compound having a heteroaromatic ring having a diazine skeleton, the organic compound having a heteroaromatic ring having a pyridine skeleton, and the organic compound having a heteroaromatic ring having a triazine skeleton have high reliability and thus are preferable. In particular, the organic compound having a heteroaromatic ring having a diazine (pyrimidine or pyrazine) skeleton and the organic compound having a heteroaromatic ring having a triazine skeleton have a high electron-transport property to contribute to a reduction in driving voltage.

The electron mobility of the electron-transport layer is preferably higher than or equal to 1×10⁻⁷ cm²/Vs and lower than or equal to 5×10⁻⁵ cm²/Vs in the case where the square root of the electric field strength [V/cm] is 600. The amount of electrons injected into the light-emitting layer can be controlled by the reduction in the electron-transport property of the electron-transport layer, whereby the light-emitting layer can be prevented from having excess electrons. It is particularly preferable to employ this structure when the hole-injection layer is formed using a composite material that includes a material having a hole-transport property with a relatively deep HOMO level higher than or equal to −5.7 eV and lower than or equal to −5.4 eV, in which case a long lifetime can be achieved. In this case, the material having an electron-transport property preferably has a HOMO level higher than or equal to −6.0 eV.

### <<Structure of Electron-Injection Layer>>

A layer containing an alkali metal, an alkaline earth metal, a rare earth metal complex, a compound thereof, or a complex thereof, such as lithium fluoride (LiF), cesium fluoride (CsF), calcium fluoride (CaF₂), 8-quinolinolato-lithium (abbreviation: Liq), or ytterbium (Yb), may be provided as the electron-injection layer 115. An electride or a layer that is formed using a substance having an electron-transport property and that contains an alkali metal, an alkaline earth metal, or a compound thereof may be used as the electron-injection layer 115. Examples of the electride include a substance in which electrons are added at high concentration to calcium oxide-aluminum oxide.

Note that as the electron-injection layer 115, it is possible to use a layer that contains a substance having an electron-transport property (preferably an organic compound having a bipyridine skeleton) and including a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device having higher external quantum efficiency can be provided.

The organic compound of one embodiment of the present invention described in Embodiment 1 can be used for the electron-injection layer 115. The electron-injection layer 115 may contain a substance having an electron-transport property in addition to the organic compound of one embodiment of the present invention described in Embodiment 1.

### <<Structure of Second Electrode>>

The second electrode 102 is an electrode including a cathode. The second electrode 102 may have a stacked-layer structure where a layer in contact with the organic compound layer 103 functions as the cathode. As a substance of the cathode, any of a metal, an alloy, and an electrically conductive compound with a low work function (specifically, lower than or equal to 3.8 eV), a mixture thereof, or the like can be used. Specific examples of such a cathode material include elements belonging to Group 1 or 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the second electrode 102 and the electron-transport layer, a variety of conductive materials such as Al, Ag, ITO, and indium oxide-tin oxide containing silicon or silicon oxide can be used for the cathode regardless of the work function.

When the second electrode 102 is formed using a material transmitting visible light, the light-emitting device can emit light from the second electrode 102 side.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Any of a variety of methods can be used for forming the organic compound layer 103, regardless of whether it is a dry process or a wet process. For example, a vacuum evaporation method, a gravure printing method, an offset printing method, a screen printing method, an ink-jet method, a spin coating method, or the like may be used.

Different film formation methods may be used to form the electrodes or the layers described above.

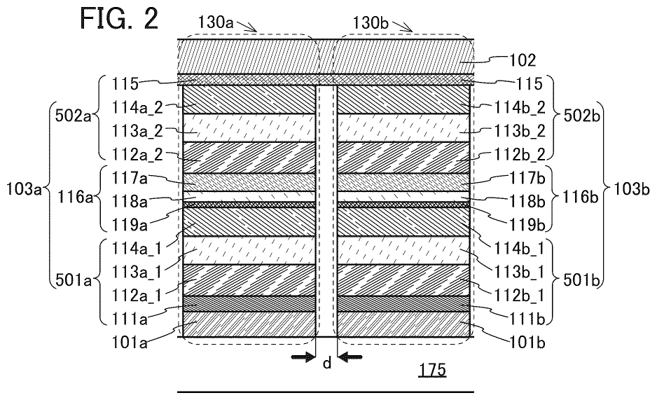

FIG. 2 illustrates two adjacent light-emitting devices (a light-emitting device 130a and a light-emitting device 130b) included in a display apparatus of one embodiment of the present invention.

The light-emitting device 130a includes an organic compound layer 103a between a first electrode 101a and the second electrode 102 over an insulating layer 175. The organic compound layer 103a has a structure where a first light-emitting unit 501a and a second light-emitting unit 502a are stacked with an intermediate layer 116a interposed therebetween. Although two light-emitting units are stacked in the example illustrated in FIG. 2, three or more light-emitting units may be stacked. The first light-emitting unit 501a includes a hole-injection layer 11a, a first hole-transport layer 112a_1, a first light-emitting layer 113a_1, and a first electron-transport layer 114a_1. The intermediate layer 116a includes a second layer 117a, a third layer 118a, and a first layer 119a. The third layer 118a is not necessarily provided. The second light-emitting unit 502a includes a second hole-transport layer 112a_2, a second light-emitting layer 113a_2, a second electron-transport layer 114a_2, and the electron-injection layer 115.

The light-emitting device 130b includes an organic compound layer 103b between a first electrode 101b and the second electrode 102 over the insulating layer 175. The organic compound layer 103b has a structure where a first light-emitting unit 501b and a second light-emitting unit 502b are stacked with an intermediate layer 116b interposed therebetween. Although two light-emitting units are stacked in the example illustrated in FIG. 2, three or more light-emitting units may be stacked. The first light-emitting unit 501b includes a hole-injection layer 111b, a first hole-transport layer 112b_1, a first light-emitting layer 113b_1, and a first electron-transport layer 114b_1. The intermediate layer 116b includes a second layer 117b, a third layer 118b, and a first layer 119b. The third layer 118b is not necessarily provided. The second light-emitting unit 502b includes a second hole-transport layer 112b_2, a second light-emitting layer 113b_2, a second electron-transport layer 114b_2, and the electron-injection layer 115.

The electron-injection layer 115 and the second electrode 102 are each preferably one continuous layer shared by the light-emitting device 130a and the light-emitting device 130b. The organic compound layer 103a and the organic compound layer 103b, except for the electron-injection layer 115, are processed by a photolithography method after the second electron-transport layer 114a_2 is formed and after the second electron-transport layer 114b_2 is formed and thus are independent of each other. Furthermore, since the end portions (outlines) of the organic compound layer 103a except for the electron-injection layer 115 are processed by a photolithography method, the end portions are substantially aligned in the direction perpendicular to the substrate. Since the end portions (outlines) of the organic compound layer 103b except for the electron-injection layer 115 are processed by a photolithography method, the end portions are substantially aligned in the direction perpendicular to the substrate.

Since the organic compound layers are processed by a photolithography method, a distance d between the first electrode 101a and the first electrode 101b can be smaller than that of the case where mask vapor deposition is performed, and can be greater than or equal to 2 μm and less than or equal to 5 μm.

This embodiment can be combined as appropriate with any of the other embodiments or examples. In this specification, in the case where a plurality of structure examples are shown in one embodiment, the structure examples can be combined as appropriate.

### Embodiment 3

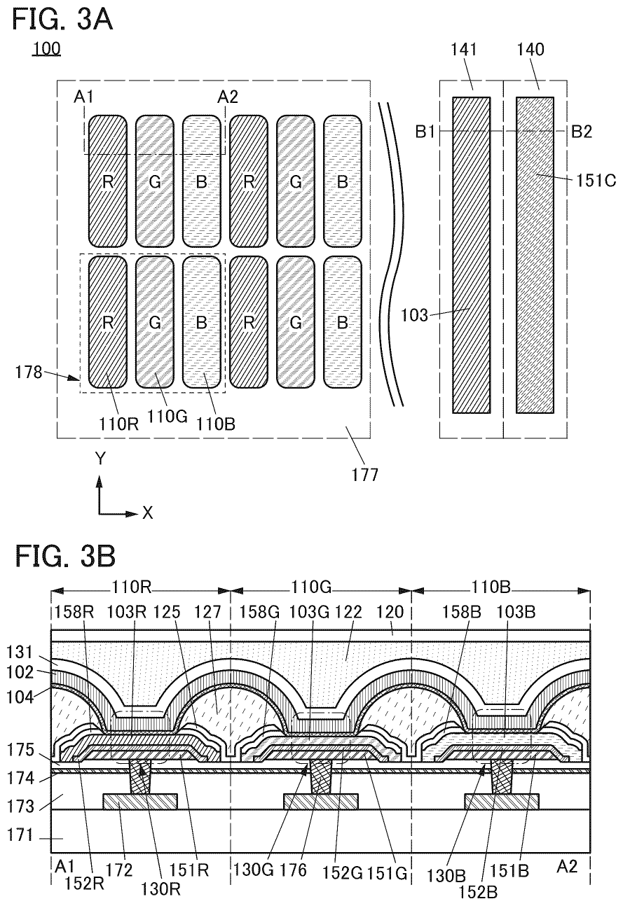

As illustrated in FIG. 3A and FIG. 3B, a plurality of the light-emitting devices 130 are formed over the insulating layer 175 to constitute a display apparatus. In this embodiment, display apparatuses of embodiments of the present invention are described in detail.

A display apparatus 100 includes a pixel portion 177 in which a plurality of pixels 178 are arranged in a matrix. The pixels 178 each include a subpixel 110R, a subpixel 110G, and a subpixel 110B.

In this specification and the like, for example, matters common to the subpixel 110R, the subpixel 110G, and the subpixel 110B are sometimes described using the collective term “subpixel 110”. In the same manner, in the description common to other components that are distinguished by alphabets, reference numerals without alphabets are sometimes used.

The subpixel 110R emits red light, the subpixel 110G emits green light, and the subpixel 110B emits blue light. Thus, an image can be displayed on the pixel portion 177. Note that in this embodiment, subpixels of three colors of red (R), green (G), and blue (B) are given as examples; however, subpixels of a different combination of colors may be employed. The number of subpixels is not limited to three and may be four or more. Examples of four subpixels include subpixels of four colors of R, G, B, and white (W), subpixels of four colors of R, G, B, and Y, and four subpixels of R, G, B, and infrared light (IR).

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are positioned on the right side of the pixel portion 177, the positions of the region 141 and the connection portion 140 are not particularly limited. The number of the regions 141 and the number of the connection portions 140 can each be one or more.

FIG. 3B is an example of a cross-sectional view taken along the dashed-dotted line A1-A2 in FIG. 3A. As illustrated in FIG. 3B, the display apparatus 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layer 175, the insulating layer 174, and the insulating layer 173, and a plug 176 is provided so as to fill the opening.

In the pixel portion 177, the light-emitting device 130 is provided over the insulating layer 175 and the plug 176. A protective layer 131 is provided to cover the light-emitting device 130. A substrate 120 is attached onto the protective layer 131 with a resin layer 122. In a region between adjacent light-emitting devices 130, an inorganic insulating layer 125 and an insulating layer 127 over the inorganic insulating layer 125 are preferably provided.

Although FIG. 3B illustrates a plurality of cross sections of the inorganic insulating layer 125 and the insulating layer 127, it is preferable that the inorganic insulating layer 125 and the insulating layer 127 be each a continuous layer when the display apparatus 100 is seen from above. In other words, the inorganic insulating layer 125 and the insulating layer 127 are preferably insulating layers having an opening portion over a first electrode.

In FIG. 3B, a light-emitting device 130R, a light-emitting device 130G, and a light-emitting device 130B are illustrated as the light-emitting device 130. The light-emitting device 130R, the light-emitting device 130G, and the light-emitting device 130B emit light of different colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

The display apparatus of one embodiment of the present invention is a top-emission display apparatus where light is emitted in the direction opposite to a substrate over which the light-emitting devices are formed. Note that the display apparatus of one embodiment of the present invention may be of a bottom-emission type.

Examples of a light-emitting substance contained in the light-emitting device 130 include organic compounds or organometallic complexes such as a substance exhibiting fluorescence (a fluorescent material), a substance exhibiting phosphorescence (a phosphorescent material), and a substance exhibiting thermally activated delayed fluorescence (a TADF material). Other examples include inorganic compounds such as quantum dots.

The light-emitting device 130R has a structure described in Embodiment 2. The light-emitting device 130R includes the first electrode (pixel electrode) including a conductive layer 151R and a conductive layer 152R, an organic compound layer 103R over the first electrode, a common layer 104 over the organic compound layer 103R, and the second electrode (common electrode) 102 over the common layer 104. Note that the common layer 104 is not necessarily provided, but is preferably provided to reduce damage to the organic compound layer 103R during processing. In the case where the common layer 104 is provided, the common layer 104 is preferably an electron-injection layer. Furthermore, in the case where the common layer 104 is provided, a stacked-layer structure of the organic compound layer 103R and the common layer 104 corresponds to the organic compound layer 103 in Embodiment 2.

The light-emitting device 130G has a structure described in Embodiment 2. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 102 over the common layer 104. Note that the common layer 104 is not necessarily provided, but is preferably provided to reduce damage to the organic compound layer 103G during processing. In the case where the common layer 104 is provided, the common layer 104 is preferably an electron-injection layer. Furthermore, in the case where the common layer 104 is provided, a stacked-layer structure of the organic compound layer 103G and the common layer 104 corresponds to the organic compound layer 103 in Embodiment 2.

The light-emitting device 130B has a structure described in Embodiment 2. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 102 over the common layer 104. Note that the common layer 104 is not necessarily provided, but is preferably provided to reduce damage to the organic compound layer 103B during processing. In the case where the common layer 104 is provided, the common layer 104 is preferably an electron-injection layer. Furthermore, in the case where the common layer 104 is provided, a stacked-layer structure of the organic compound layer 103B and the common layer 104 corresponds to the organic compound layer 103 in Embodiment 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, the organic compound layers 103G, and the organic compound layers 103B are island-shaped layers isolated on a light-emitting device basis or on an emission color basis. Providing the island-shaped organic compound layer 103 in each of the light-emitting devices 130 can inhibit leakage current between the adjacent light-emitting devices 130 even in a high-resolution display apparatus. This can prevent crosstalk, so that the display apparatus can achieve extremely high contrast. Specifically, a display apparatus having high current efficiency at low luminance can be obtained.

The island-shaped organic compound layer 103 is formed by forming an EL film and processing the EL film by a lithography method.

The organic compound layer 103 is preferably provided to cover the top surface and the side surface of the first electrode (pixel electrode) of the light-emitting device 130. Such a structure can easily increase the aperture ratio of the display apparatus 100 as compared with the structure where an end portion of the organic compound layer 103 is positioned inward from an end portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited. Furthermore, the distance between a light-emitting region (i.e., a region overlapping with the pixel electrode) in the organic compound layer 103 and the end portion of the organic compound layer 103 can be increased. Since the end portion of the organic compound layer 103 might be damaged by processing, the use of a region away from the end portion of the organic compound layer 103 as the light-emitting region can improve the reliability of the light-emitting device 130.

In the display apparatus of one embodiment of the present invention, the first electrode (pixel electrode) of the light-emitting device preferably has a stacked-layer structure. For example, in the example illustrated in FIG. 3B, the first electrode of the light-emitting device 130 has a stacked-layer structure of a conductive layer 151 and a conductive layer 152. In the case where the display apparatus 100 has a top-emission structure and the pixel electrode of the light-emitting device 130 functions as an anode, for example, it is preferable that the conductive layer 151 have high visible light reflectance and the conductive layer 152 have a visible-light-transmitting property and a high work function. In the case where the display apparatus 100 is of a top-emission type, the higher the visible light reflectance of the pixel electrode is, the higher the efficiency of extraction of the light emitted by the organic compound layer 103 can be. In the case where the pixel electrode functions as an anode, the higher the work function of the pixel electrode is, the easier it is to inject holes into the organic compound layer 103. Thus, when the pixel electrode of the light-emitting device 130 has a stacked-layer structure of the conductive layer 151 with high visible light reflectance and the conductive layer 152 with a high work function, the light-emitting device 130 can have high light extraction efficiency and a low driving voltage.

In the case where the conductive layer 151 has high visible light reflectance, the visible light reflectance of the conductive layer 151 is preferably higher than or equal to 40% and lower than or equal to 100%, further preferably higher than or equal to 70% and lower than or equal to 100%, for example. When used as an electrode having a visible-light-transmitting property, the conductive layer 152 preferably has a visible light transmittance higher than or equal to 40%, for example.

Here, in the case of having a stacked-layer structure of a plurality of layers, the pixel electrode might change in quality as a result of a reaction between the plurality of layers, for example. For example, when a film formed after the formation of the pixel electrode is removed by a wet etching method, contact of a chemical solution with the pixel electrode might cause galvanic corrosion.

In view of the above, the conductive layer 152 is formed to cover the top surface and the side surface of the conductive layer 151 in the display apparatus 100 of this embodiment. This can inhibit the chemical solution from coming into contact with the conductive layer 151 even in the case where a film formed after formation of the pixel electrode including the conductive layer 151 and the conductive layer 152 is removed by a wet etching method, for example. Thus, occurrence of galvanic corrosion to the pixel electrode can be inhibited, for example. Thus, since the display apparatus 100 can be manufactured by a method giving a high yield, an inexpensive display apparatus can be obtained. In addition, generation of a defect in the display apparatus 100 can be inhibited, which makes the display apparatus 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, an indium tin oxide, an indium zinc oxide, zinc oxide, zinc oxide containing gallium, titanium oxide, an indium zinc oxide containing gallium, an indium zinc oxide containing aluminum, an indium tin oxide containing silicon, an indium zinc oxide containing silicon, and the like. In particular, an indium tin oxide containing silicon can be suitably used for the conductive layer 152 because of having a high work function, for example, a work function higher than or equal to 4.0 eV.

The conductive layer 151 may have a stacked-layer structure of a plurality of layers including different materials and the conductive layer 152 may have a stacked-layer structure of a plurality of layers including different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 has a stacked-layer structure of two or more layers, for example, a layer in contact with the conductive layer 152 can be formed using a material that can be used for the conductive layer 152.

The conductive layer 151 preferably has an end portion with a tapered shape. Specifically, the end portion of the conductive layer 151 preferably has a tapered shape with a taper angle of less than 90°. In that case, the conductive layer 152 provided along the side surface of the conductive layer 151 also has a tapered shape. When the side surface of the conductive layer 152 has a tapered shape, coverage with the organic compound layer 103 provided along the side surface of the conductive layer 152 can be improved.

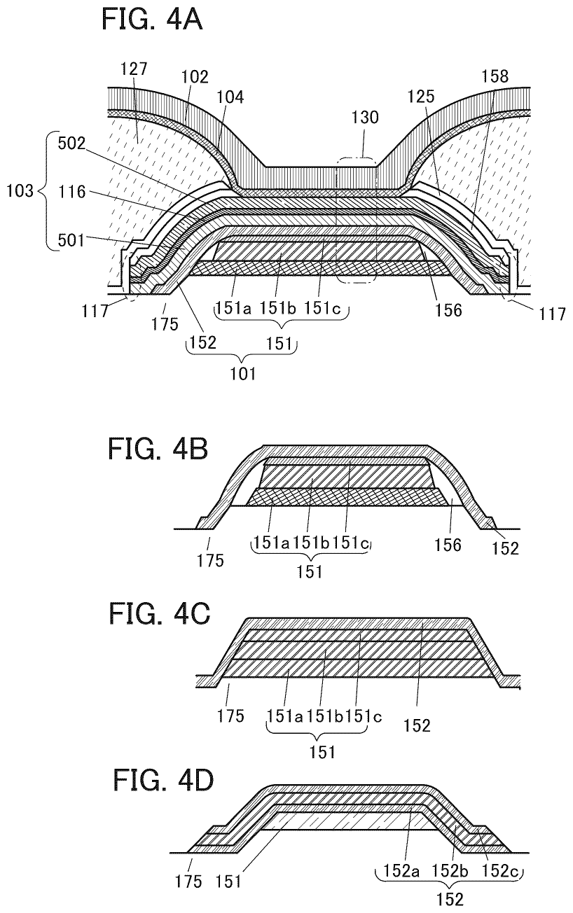

FIG. 4A illustrates the case where the conductive layer 151 has a stacked-layer structure of a plurality of layers including different materials. As illustrated in FIG. 4A, the conductive layer 151 includes a conductive layer 151a, a conductive layer 151b over the conductive layer 151a, and a conductive layer 151c over the conductive layer 151b. In other words, the conductive layer 151 illustrated in FIG. 4A has a three-layer stacked structure. In the case where the conductive layer 151 has a stacked-layer structure of a plurality of layers as described above, the visible light reflectance of at least one of the layers included in the conductive layer 151 is higher than that of the conductive layer 152.

In the example illustrated in FIG. 4A, the conductive layer 151b is interposed between the conductive layer 151a and the conductive layer 151c. A material that is less likely to change in quality than a material for the conductive layer 151b is preferably used for the conductive layer 151a and the conductive layer 151c. For example, a material that is less likely to cause migration due to contact with the insulating layer 175 than the material for the conductive layer 151b can be used for the conductive layer 151a. For the conductive layer 151c, a material that is less likely to be oxidized than the material for the conductive layer 151b and that forms an oxide having lower electrical resistivity than an oxide of the material for the conductive layer 151b can be used.

In this manner, the structure where the conductive layer 151b is interposed between the conductive layer 151a and the conductive layer 151c can expand the range of choices for the material for the conductive layer 151b. The conductive layer 151b, for example, can thus have higher visible light reflectance than at least one of the conductive layer 151a and the conductive layer 151c. For example, aluminum can be used for the conductive layer 151b. Note that an alloy containing aluminum may be used for the conductive layer 151b. For the conductive layer 151a, titanium, a material which has lower visible light reflectance than aluminum and is less likely to cause migration even at the time of contact with the insulating layer 175 than aluminum, can be used. For the conductive layer 151c, it is possible to use titanium, a material which has lower visible light reflectance than aluminum and is less likely to be oxidized than aluminum and whose oxide has lower electrical resistivity than aluminum oxide.

For the conductive layer 151c, silver or an alloy containing silver may be used. Silver is characterized by its visible light reflectance higher than that of titanium. In addition, silver is characterized by being less likely to be oxidized than aluminum, and silver oxide is characterized by its electrical resistivity lower than that of aluminum oxide. Thus, the use of silver or an alloy containing silver for the conductive layer 151c can suitably increase the visible light reflectance of the conductive layer 151 and inhibit an increase in the electrical resistance of the pixel electrode due to oxidation of the conductive layer 151b. Here, as the alloy containing silver, an alloy of silver, palladium, and copper (also referred to as Ag—Pd—Cu or APC) can be used, for example. When the conductive layer 151c is formed using silver or an alloy containing silver and the conductive layer 151b is formed using aluminum, the visible light reflectance of the conductive layer 151c can be higher than that of the conductive layer 151b. Here, the conductive layer 151b may be formed using silver or an alloy containing silver. The conductive layer 151a may be formed using silver or an alloy containing silver.

Meanwhile, a film formed using titanium has better processability in etching than a film formed using silver. Thus, the use of titanium for the conductive layer 151c facilitates the formation of the conductive layer 151c. Note that a film formed using aluminum also has better processability in etching than a film formed using silver.

The conductive layer 151 having a stacked-layer structure of a plurality of layers as described above can improve the characteristics of the display apparatus. For example, the display apparatus 100 can have high light extraction efficiency and high reliability.

Here, in the case where the light-emitting device 130 has a microcavity structure, the use of silver or an alloy containing silver, which is a material having high visible light reflectance, for the conductive layer 151c can suitably increase the light extraction efficiency of the display apparatus 100.

As described above, the side surface of the conductive layer 151 preferably has a tapered shape. Specifically, the side surface of the conductive layer 151 preferably has a tapered shape with a taper angle less than 90°. For example, in the conductive layer 151 illustrated in FIG. 4A, the side surface of at least one of the conductive layer 151a, the conductive layer 151b, and the conductive layer 151c preferably has a tapered shape.

The conductive layer 151 illustrated in FIG. 4A can be formed by a photolithography method. Specifically, first, a conductive film to be the conductive layer 151a, a conductive film to be the conductive layer 151b, and a conductive film to be the conductive layer 151c are sequentially formed. Next, a resist mask is formed over the conductive film to be the conductive layer 151c. Then, the conductive films in a region not overlapping with the resist mask are removed by an etching method, for example. Here, the side surface of the conductive layer 151 can have a tapered shape by processing the conductive films under conditions where the resist mask is easily recessed (reduced in size) as compared with the case where the conductive layer 151 is formed such that the side surface does not have a tapered shape, i.e., a perpendicular side surface is formed.

Here, when the conductive film is processed under conditions where the resist mask is easily recessed (reduced in size), the conductive film might be easily processed in the horizontal direction. That is, the etching sometimes might become isotropic as compared with the case where the conductive layer 151 is formed to have a perpendicular side surface.

In the case where the conductive layer 151 has a stacked-layer structure of a plurality of layers formed of different materials, the plurality of layers sometimes differ in processability in the horizontal direction. For example, the conductive layer 151a, the conductive layer 151b, and the conductive layer 151c sometimes differ in processability in the horizontal direction.

In that case, after the processing of the conductive film, as illustrated in FIG. 4A, the side surface of the conductive layer 151b may be positioned inward from the side surfaces of the conductive layer 151a and the conductive layer 151c and a protruding portion may be formed. This might impair coverage of the conductive layer 151 with the conductive layer 152 to cause step disconnection of the conductive layer 152.

In view of this, an insulating layer 156 is preferably provided as illustrated in FIG. 4A. FIG. 4A illustrates an example where the insulating layer 156 is provided over the conductive layer 151a to include a region overlapping with the side surface of the conductive layer 151b. Such a structure can inhibit occurrence of the step disconnection or a reduction in the thickness of the conductive layer 152 due to the protruding portion; thus, connection defects or an increase in driving voltage can be inhibited.

Although FIG. 4A illustrates the structure where the side surface of the conductive layer 151b is entirely covered with the insulating layer 156, part of the side surface of the conductive layer 151b is not necessarily covered with the insulating layer 156. Also in a pixel electrode with a later-described structure, part of the side surface of the conductive layer 151b is not necessarily covered with the insulating layer 156.

In the case where the conductive layer 151 has the structure illustrated in FIG. 4A, the conductive layer 152 is provided to cover the conductive layer 151a, the conductive layer 151b, and the conductive layer 151c and the insulating layer 156 and to be electrically connected to the conductive layer 151a, the conductive layer 151b, and the conductive layer 151c. This can prevent a chemical solution from coming into contact with the conductive layer 151a, the conductive layer 151b, and the conductive layer 151c even when a film formed after formation of the conductive layer 152 is removed by a wet etching method, for example. It is thus possible to inhibit occurrence of corrosion in the conductive layer 151a, the conductive layer 151b, and the conductive layer 151c. As described above, the display apparatus 100 can be manufactured by a high-yield method. In addition, generation of a defect can be inhibited, which makes the display apparatus 100 highly reliable.

Here, the insulating layer 156 preferably has a curved surface as illustrated in FIG. 4A. In this case, step disconnection of the conductive layer 152 covering the insulating layer 156 is less likely to occur than in the case where the insulating layer 156 has a perpendicular side surface (a side surface parallel to the Z direction), for example. In addition, step disconnection of the conductive layer 152 covering the insulating layer 156 is less likely to occur also in the case where the side surface of the insulating layer 156 has a tapered shape, specifically, a tapered shape with a taper angle less than 90°, than in the case where the insulating layer 156 has a perpendicular side surface, for example. As described above, the display apparatus 100 can be manufactured by a high-yield method. In addition, generation of a defect can be inhibited, which makes the display apparatus 100 highly reliable.

FIG. 4A illustrates the structure where the side surface of the conductive layer 151b is positioned inward from that of the conductive layer 151a and that of the conductive layer 151c; however, one embodiment of the present invention is not limited thereto. For example, the side surface of the conductive layer 151b may be positioned outward from that of the conductive layer 151a. The side surface of the conductive layer 151b may be positioned outward from that of the conductive layer 151c.

FIG. 4B to FIG. 4D illustrate other structures of the first electrode 101. FIG. 4B illustrates a structure of the first electrode 101 in FIG. 4A, in which the insulating layer 156 covers the side surfaces of the conductive layer 151a, the conductive layer 151b, and the conductive layer 151c instead of covering only the side surface of the conductive layer 151b.

FIG. 4C illustrates a structure of the first electrode 101 in FIG. 4A, in which the insulating layer 156 is not provided.

FIG. 4D illustrates a structure of the first electrode 101 in FIG. 4A, in which the conductive layer 151 does not have a stacked-layer structure and the conductive layer 152 has a stacked-layer structure.

A conductive layer 152a has higher adhesion to the conductive layer 152b than the insulating layer 175 does, for example. For the conductive layer 152a, an oxide containing one or more selected from indium, tin, zinc, gallium, titanium, aluminum, and silicon can be used. For example, it is preferable to use a conductive oxide containing one or more of indium oxide, an indium tin oxide, an indium zinc oxide, zinc oxide, zinc oxide containing gallium, titanium oxide, an indium titanium oxide, zinc titanate, an aluminum zinc oxide, an indium zinc oxide containing gallium, an indium zinc oxide containing aluminum, an indium tin oxide containing silicon, an indium zinc oxide containing silicon, and the like. Accordingly, peeling of the conductive layer 152b can be inhibited. The conductive layer 152b is not in contact with the insulating layer 175.

The conductive layer 152b is a layer whose visible light reflectance (e.g., reflectance with respect to light with a predetermined wavelength longer than or equal to 400 nm and shorter than 750 nm) is higher than that of the conductive layer 151, the conductive layer 152a, and a conductive layer 152c. The visible light reflectance of the conductive layer 152b can be, for example, higher than or equal to 70% and lower than or equal to 100%, and is preferably higher than or equal to 80% and lower than or equal to 100%, further preferably higher than or equal to 90% and lower than or equal to 100%. For the conductive layer 152b, a material having higher visible light reflectance than aluminum can be used, for example. Specifically, for the conductive layer 152b, silver or an alloy containing silver can be used, for example. An example of the alloy containing silver is an alloy of silver, palladium, and copper (APC). Consequently, the display apparatus 100 can be a display apparatus with high light extraction efficiency. Note that a metal other than silver may be used for the conductive layer 152b.

In the case where the conductive layer 151 and the conductive layer 152 function as the anode, the conductive layer 152c is preferably a layer having a high work function. The conductive layer 152c is a layer having a higher work function than the conductive layer 152b, for example. For the conductive layer 152c, a material similar to the material that can be used for the conductive layer 152a can be used, for example. For example, the conductive layer 152a and the conductive layer 152c can be formed using the same kind of material. For example, in the case where an indium tin oxide is used for the conductive layer 152a, an indium tin oxide can also be used for the conductive layer 152c.

In the case where the conductive layer 151 and the conductive layer 152 function as the cathode, the conductive layer 152c is preferably a layer having a low work function. The conductive layer 152c is a layer having a lower work function than the conductive layer 152b, for example.

The conductive layer 152c is preferably a layer having high visible light transmittance (e.g., transmittance with respect to light with a predetermined wavelength longer than or equal to 400 nm and shorter than 750 nm). For example, the visible light transmittance of the conductive layer 152c is preferably higher than those of the conductive layer 151 and the conductive layer 152b. The visible light transmittance of the conductive layer 152c can be, for example, higher than or equal to 60% and lower than or equal to 100%, and is preferably higher than or equal to 70% and lower than or equal to 100%, further preferably higher than or equal to 80% and lower than or equal to 100%. In that case, the amount of light absorbed by the conductive layer 152c after being emitted from the organic compound layer 103 can be reduced. As described above, the conductive layer 152b under the conductive layer 152c can be a layer having high visible light reflectance. Thus, the display apparatus 100 can have high light extraction efficiency.

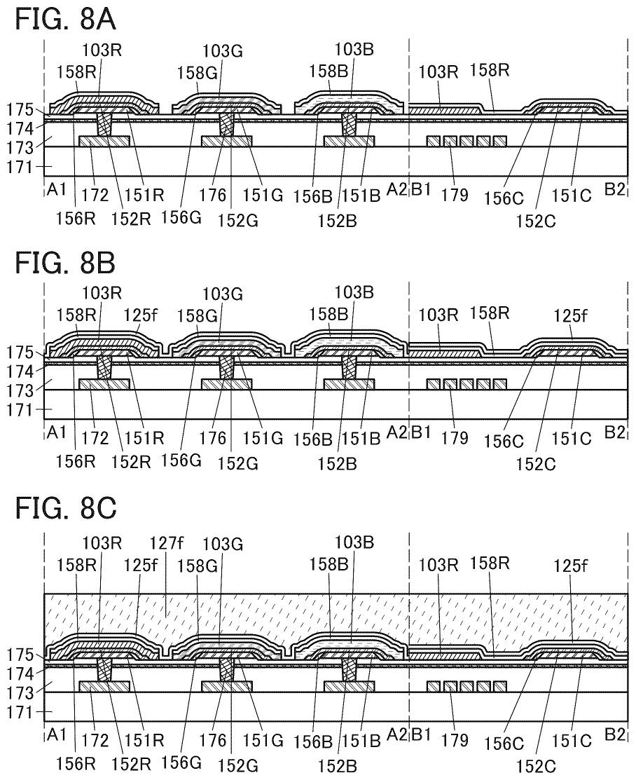

Next, an example of a method for manufacturing the display apparatus 100 having the structure illustrated in FIG. 3A is described with reference to FIG. 8 to FIG. 16. An organic layer of the light-emitting device included in the display apparatus 100 is formed by a manufacturing process including treatment using water. The use of the organic compound of one embodiment of the present invention for the organic layer of the light-emitting device included in the display apparatus of one embodiment of the present invention prevents problems such as dissolution of the layer including the organic compound and permeation of a chemical solution into the layer including the organic compound even when the manufacturing method including treatment using water is employed; consequently, the light-emitting device can have favorable characteristics.

### Manufacturing Method Example

Thin films included in the display apparatus (insulating films, semiconductor films, conductive films, and the like) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic chemical vapor deposition (MOCVD: Metal Organic CVD) method.

The thin films included in the display apparatus (insulating films, semiconductor films, conductive films, and the like) can be formed by a wet film formation method such as spin coating, dipping, spray coating, inkjetting, dispensing, screen printing, offset printing, a doctor knife method, slit coating, roll coating, curtain coating, or knife coating.

Specifically, for manufacture of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display apparatus can be processed by, for example, a photolithography method. Alternatively, a nanoimprinting method, a sandblasting method, a lift-off method, or the like may be used to process the thin films. Alternatively, island-shaped thin films may be directly formed by a film formation method using a shielding mask such as a metal mask.

As the lithography method, a photolithography method can be used, for example. There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for light exposure in a photolithography method, for example, an i-line (with a wavelength of 365 nm), a g-line (with a wavelength of 436 nm), an h-line (with a wavelength of 405 nm), or light in which these lines are mixed can be used. Besides, ultraviolet rays, KrF laser light, ArF laser light, or the like can be used. The light exposure may be performed by liquid immersion exposure technique. As the light used for light exposure, extreme ultraviolet (EUV) light or X-rays may be used. Instead of the light used for the light exposure, an electron beam can be used. It is preferable to use extreme ultraviolet light, X-rays, or an electron beam because extremely minute processing is possible. Note that when light exposure is performed by scanning with a beam such as an electron beam, a photomask is not needed.

For etching of thin films, a dry etching method, a wet etching method, a sandblast method, or the like can be used.

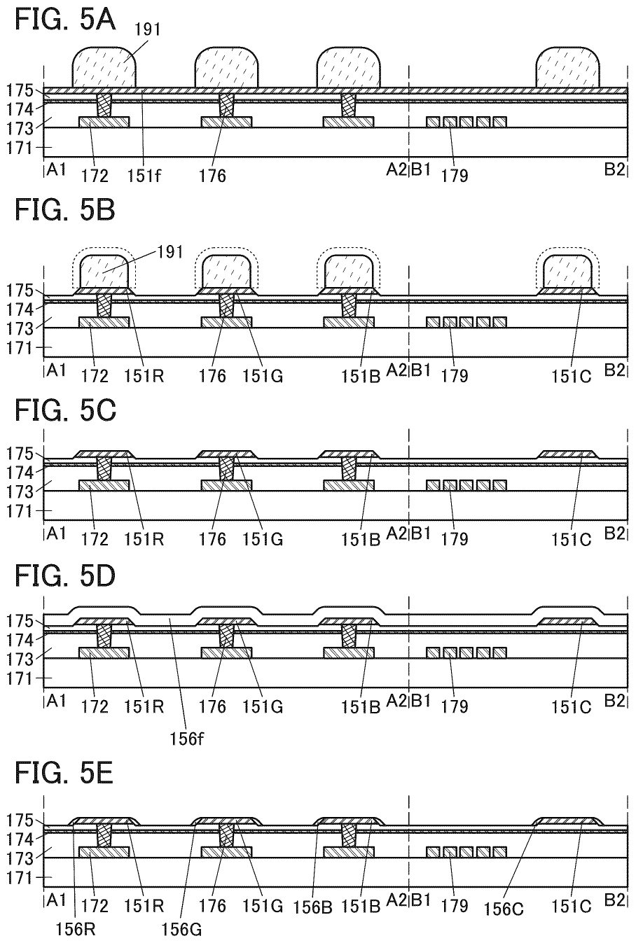

First, the insulating layer 171 is formed over a substrate (not illustrated), as illustrated in FIG. 5A. Next, the conductive layer 172 and a conductive layer 179 are formed over the insulating layer 171, and the insulating layer 173 is formed over the insulating layer 171 to cover the conductive layer 172 and the conductive layer 179. Then, the insulating layer 174 is formed over the insulating layer 173, and the insulating layer 175 is formed over the insulating layer 174.

As the substrate, a substrate having heat resistance high enough to withstand at least heat treatment performed later can be used. When an insulating substrate is used as the substrate, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like; a compound semiconductor substrate of silicon germanium or the like; or an SOI substrate.

Next, openings reaching the conductive layer 172 are formed in the insulating layer 175, the insulating layer 174, and the insulating layer 173, as illustrated in FIG. 5A. Then, the plugs 176 are formed to fill the openings.

Next, a conductive film 151f to be the conductive layer 151R, the conductive layer 151G, the conductive layer 151B, and the conductive layer 151C later is formed over the plugs 176 and the insulating layer 175, as illustrated in FIG. 5A. For formation of the conductive film 151f, a sputtering method or a vacuum evaporation method can be used, for example. A metal material can be used for the conductive film 151f, for example.

Next, as illustrated in FIG. 5A, a resist mask 191 is formed over the conductive film 151f. The resist mask 191 can be formed by application of a photosensitive material (photoresist), light exposure, and development.

Subsequently, as illustrated in FIG. 5B, the conductive film 151f in a region not overlapping with the resist mask 191, for example, is removed by an etching method, specifically a dry etching method, for example. Note that in the case where the conductive film 151f includes a layer formed using a conductive oxide such as an indium tin oxide, for example, the layer may be removed by a wet etching method. In this manner, the conductive layer 151 is formed. Here, in the case where part of the conductive film 151f is removed by a dry etching method, for example, a depressed portion may be formed in a region of the insulating layer 175 not overlapping with the conductive layer 151.

Next, as illustrated in FIG. 5C, the resist mask 191 is removed. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Next, as illustrated in FIG. 5D, an insulating film 156f to be an insulating layer 156R, an insulating layer 156G, an insulating layer 156B, and an insulating layer 156C later is formed over the conductive layer 151R, the conductive layer 151G, the conductive layer 151B, the conductive layer 151C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film containing silicon, a nitride insulating film containing silicon, an oxynitride insulating film containing silicon, a nitride oxide insulating film containing silicon, or the like can be used as the insulating film 156f. For the insulating film 156f, silicon oxynitride can be used, for example.

Subsequently, as illustrated in FIG. 5E, the insulating film 156f is processed to form the insulating layer 156R, the insulating layer 156G, the insulating layer 156B, and the insulating layer 156C. The insulating layer 156 can be formed by performing etching substantially uniformly on the top surface of the insulating film 156f, for example. Such uniform etching for planarization is also referred to as etch-back processing. Note that the insulating layer 156 may be formed by a lithography method.

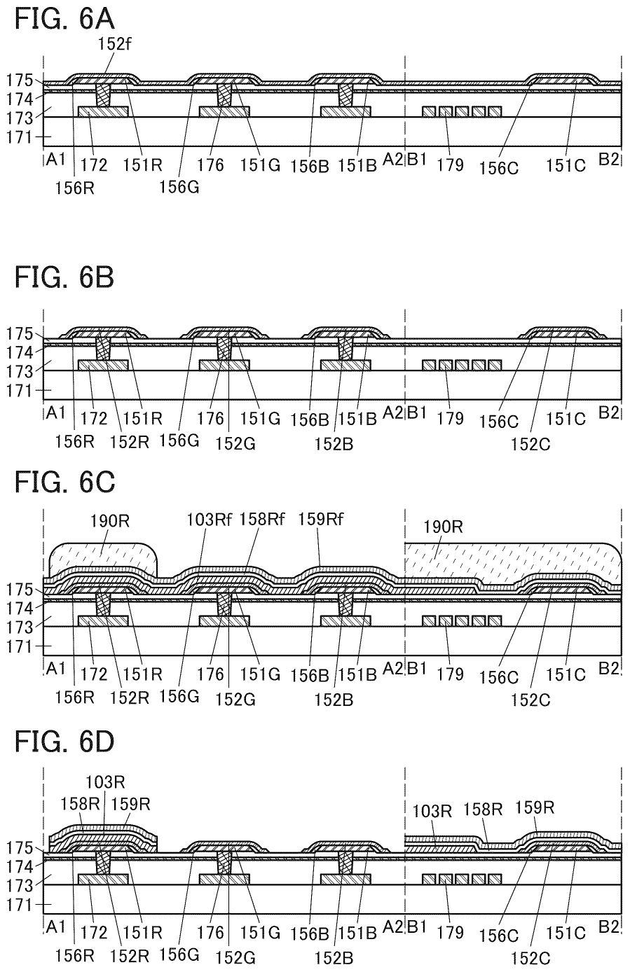

Then, as illustrated in FIG. 6A, a conductive film 152f to be the conductive layer 152R, the conductive layer 152G, the conductive layer 152B, and a conductive layer 152C later is formed over the conductive layer 151R, the conductive layer 151G, the conductive layer 151B, the conductive layer 151C, the insulating layer 156R, the insulating layer 156G, the insulating layer 156B, the insulating layer 156C, and the insulating layer 175. Specifically, the conductive film 152f is formed to cover the conductive layer 151R, the conductive layer 151G, the conductive layer 151B, the conductive layer 151C, the insulating layer 156R, the insulating layer 156G, the insulating layer 156B, and the insulating layer 156C, for example.

The conductive film 152f can be formed by a sputtering method or a vacuum evaporation method, for example. A conductive oxide can be used for the conductive film 152f, for example. The conductive film 152f can have a stacked-layer structure of a film formed using a metal material and a film formed thereover using a conductive oxide. For example, the conductive film 152f can have a stacked-layer structure of a film formed using titanium, silver, or an alloy containing silver and a film formed thereover using a conductive oxide.

The conductive film 152f can be formed by an ALD method. In that case, an oxide containing one or more selected from indium, tin, zinc, gallium, titanium, aluminum, and silicon can be used for the conductive film 152f. In that case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film containing a plurality of kinds of metals, such as an indium tin oxide, is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In—O film, and then a precursor containing tin is introduced, the precursor is purged, and an oxidizer is introduced to form a Sn—O film. Here, when the number of cycles of forming an In—O film is larger than the number of cycles of forming a Sn—O film, the number of In atoms contained in the conductive film 152f can be larger than the number of Sn atoms contained therein.

For example, to form a zinc oxide film as the conductive film 152f, a Zn—O film is formed in the above procedure. For example, to form an aluminum zinc oxide film as the conductive film 152f, a Zn—O film and an Al—O film are formed in the above procedure. For example, to form a titanium oxide film as the conductive film 152f, a Ti—O film is formed in the above procedure. For example, to form an indium tin oxide film containing silicon as the conductive film 152f, an In—O film, a Sn—O film, and a Si—O film are formed in the above procedure. For example, to form a zinc oxide film containing gallium, a Ga—O film and a Zn—O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Then, as illustrated in FIG. 6B, the conductive film 152f is processed by a photolithography method, for example, so that the conductive layer 152R, the conductive layer 152G, the conductive layer 152B, and the conductive layer 152C are formed. Specifically, the conductive film 152f is partly removed by an etching method after a resist mask is formed, for example. The conductive film 152f can be removed by a wet etching method, for example. The conductive film 152f may be removed by a dry etching method. Through the above steps, the pixel electrode including the conductive layer 151 and the conductive layer 152 is formed.

Next, hydrophobization treatment for the conductive layer 152 is preferably performed. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and inhibit film peeling. Note that the hydrophobization treatment is not necessarily performed.

Next, as illustrated in FIG. 6C, an organic compound film 103Rf to be the organic compound layer 103R later is formed over the conductive layer 152R, the conductive layer 152G, the conductive layer 152B, and the insulating layer 175.

As illustrated in FIG. 6C, the organic compound film 103Rf is not formed over the conductive layer 152C. The organic compound film 103Rf can be formed only in an intended region by using a mask for specifying a film formation area (also referred to as an area mask or a rough metal mask to be distinguished from a fine metal mask), for example. Employing a film formation step using an area mask and a processing step using a resist mask enables a light-emitting device to be manufactured by a relatively easy process.

The organic compound film 103Rf can be formed by an evaporation method, specifically a vacuum evaporation method, for example. The organic compound film 103Rf may be formed by a transfer method, a printing method, an inkjet method, a coating method, or the like.

Next, as illustrated in FIG. 6C, a sacrificial film 158Rf to be a sacrificial layer 158R later and a mask film 159Rf to be a mask layer 159R later are sequentially formed over the organic compound film 103Rf, the conductive layer 152C, and the insulating layer 175.

Although this embodiment describes an example where the mask film is formed with a two-layer structure of the sacrificial film 158Rf and the mask film 159Rf, the mask film may have a single-layer structure or a stacked-layer structure of three or more layers. In addition, in this specification and the like, the mask layer may be referred to as a sacrificial layer.

The sacrificial layer provided over the organic compound film 103Rf can reduce damage to the organic compound film 103Rf in the manufacturing process of the display apparatus, increasing the reliability of the light-emitting device.

As the sacrificial film 158Rf, a film that is highly resistant to the processing conditions for the organic compound film 103Rf, specifically, a film having high etching selectivity with the organic compound film 103Rf is used. As the mask film 159Rf, a film having high etching selectivity with the sacrificial film 158Rf is used.

The sacrificial film 158Rf and the mask film 159Rf are formed at a temperature lower than the upper temperature limit of the organic compound film 103Rf The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

As the sacrificial film 158Rf and the mask film 159Rf, it is preferable to use a film that can be removed by a wet etching method. Using a wet etching method can reduce damage to the organic compound film 103Rf in processing the sacrificial film 158Rf and the mask film 159Rf, as compared with the case of using a dry etching method.

The sacrificial film 158Rf and the mask film 159Rf can be formed by a sputtering method, an ALD method (a thermal ALD method or a PEALD method), a CVD method, or a vacuum evaporation method, for example. Alternatively, the above-described wet process may be used for the formation.

Note that the sacrificial film 158Rf, which is formed over and in contact with the organic compound film 103Rf, is preferably formed by a formation method that causes less damage to the organic compound film 103Rf than a formation method of the mask film 159Rf. For example, the sacrificial film 158Rf is preferably formed by an ALD method or a vacuum evaporation method rather than a sputtering method.

As the sacrificial film 158Rf and the mask film 159Rf, it is possible to use one or more of a metal film, an alloy film, a metal oxide film, a semiconductor film, an organic insulating film, and an inorganic insulating film, for example.

For the sacrificial film 158Rf and the mask film 159Rf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material including any of the metal materials, for example. It is particularly preferable to use a low-melting-point material such as aluminum or silver. The use of a metal material capable of blocking ultraviolet rays for one or both of the sacrificial film 158Rf and the mask film 159Rf is preferable, in which case the organic compound film 103Rf can be inhibited from being irradiated with ultraviolet rays and deteriorating.

For each of the sacrificial film 158Rf and the mask film 159Rf, it is possible to use a metal oxide such as an In—Ga—Zn oxide, indium oxide, an In—Zn oxide, an In—Sn oxide, an indium titanium oxide (In—Ti oxide), an indium tin zinc oxide (In—Sn—Zn oxide), an indium titanium zinc oxide (In—Ti—Zn oxide), an indium gallium tin zinc oxide (In—Ga—Sn—Zn oxide), or an indium tin oxide containing silicon.

In addition, in place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

As each of the sacrificial film and the mask film, a film containing a material having a light-blocking property, particularly with respect to ultraviolet rays, is preferably used. Although a variety of materials such as a metal, an insulator, a semiconductor, and a metalloid that have a property of blocking ultraviolet rays can be used as the material having a light-blocking property, each of the sacrificial film and the mask film is preferably a film capable of being processed by etching and is particularly preferably a film having good processability because part or the whole of each of the sacrificial film and the mask film is removed in a later step.

For example, a semiconductor material with excellent compatibility with a semiconductor manufacturing process, such as silicon or germanium, is preferably used for the sacrificial film and the mask film. Alternatively, an oxide or a nitride of the semiconductor material can be used. Alternatively, a non-metallic material such as carbon and the like or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

The use of a film containing a material having a property of blocking ultraviolet rays as each of the sacrificial film and the mask film can inhibit the organic compound layer from being irradiated with ultraviolet rays in alight exposure step, for example. The organic compound layer is inhibited from being damaged by ultraviolet rays, so that the reliability of the light-emitting device can be improved.

Note that the same effect is obtained when a film containing a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As each of the sacrificial film 158Rf and the mask film 159Rf, an aluminum oxide film can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and the inorganic insulating layer 125 that is to be formed later. For example, an aluminum oxide film formed by an ALD method can be used for both the sacrificial film 158Rf and the inorganic insulating layer 125. Here, for the sacrificial film 158Rf and the inorganic insulating layer 125, the same film formation condition may be used or different film formation conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, the sacrificial film 158Rf is a layer most or all of which is to be removed in a later step, and thus is preferably easy to process. Therefore, the sacrificial film 158Rf is preferably formed at a substrate temperature lower than that for formation of the inorganic insulating layer 125.

An organic material may be used for one or both of the sacrificial film 158Rf and the mask film 159Rf. For example, as the organic material, a material that can be dissolved in a solvent chemically stable with respect to at least the uppermost film of the organic compound film 103Rf may be used. Specifically, a material that is dissolved in water or alcohol can be suitably used. In forming a film of such a material, it is preferable to apply the material dissolved in a solvent such as water or alcohol by a wet film formation method and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

For each of the sacrificial film 158Rf and the mask film 159Rf, an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluororesin such as perfluoropolymer may be used.

For example, an organic film (e.g., a PVA film) formed by an evaporation method or the above wet film formation method can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 6C. The resist mask 190R can be formed by application of a photosensitive material (photoresist), light exposure, and development.

Either a positive resist material or a negative resist material may be used to form the resist mask 190R.

The resist mask 190R is provided at a position overlapping with the conductive layer 152R. Note that the resist mask 190R is preferably provided also at a position overlapping with the conductive layer 152C. This can inhibit the conductive layer 152C from being damaged during the manufacturing process of the display apparatus. Note that the resist mask 190R is not necessarily provided over the conductive layer 152C. The resist mask 190R is preferably provided to cover the area from the end portion of the organic compound film 103Rf to the end portion of the conductive layer 152C (the end portion on the organic compound film 103Rf side), as illustrated in the cross-sectional view along B1-B2 in FIG. 6C.

Subsequently, as illustrated in FIG. 6D, part of the mask film 159Rf is removed using the resist mask 190R, whereby the mask layer 159R is formed. The mask layer 159R remains over the conductive layer 152R and over the conductive layer 152C. After that, the resist mask 190R is removed. Then, part of the sacrificial film 158Rf is removed using the mask layer 159R as a mask (also referred to as a hard mask), whereby the sacrificial layer 158R is formed.

The sacrificial film 158Rf and the mask film 159Rf can be processed by a wet etching method or a dry etching method. The sacrificial film 158Rf and the mask film 159Rf are preferably processed by isotropic etching.

Using a wet etching method can reduce damage to the organic compound film 103Rf in processing the sacrificial film 158Rf and the mask film 159Rf, as compared with the case of using a dry etching method. In the case of using a wet etching method, it is preferable to use a developer, a tetramethylammonium hydroxide aqueous solution (TMAH), dilute hydrofluoric acid, oxalic acid, phosphoric acid, acetic acid, nitric acid, or a chemical solution containing a mixed solution of any of these acids, for example.

Since the organic compound film 103Rf is not exposed in processing the mask film 159Rf, the range of choices for the processing method is wider than that for processing the sacrificial film 158Rf Specifically, even in the case where a gas containing oxygen is used as the etching gas in processing the mask film 159Rf, deterioration of the organic compound film 103Rf can be inhibited.

In the case of using a dry etching method for processing the sacrificial film 158Rf, deterioration of the organic compound film 103Rf can be inhibited by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using a combination of CHF₃ and He or a combination of CHF₃, He, and CH₄. In the case where an In—Ga—Zn oxide film formed by a sputtering method is used as the mask film 159Rf, part of the mask film 159Rf can be removed by a wet etching method using a diluted phosphoric acid. Alternatively, part of the mask film 159Rf may be removed by a dry etching method using CH₄ and Ar. Alternatively, part of the mask film 159Rf can be removed by a wet etching method using a diluted phosphoric acid. In the case where a tungsten film formed by a sputtering method is used as the mask film 159Rf, part of the mask film 159Rf can be removed by a dry etching method using SF₆, a combination of CF₄ and O₂, or a combination of CF₄, Cl₂, and O₂.

The resist mask 190R can be removed by a method similar to that for the resist mask 191. The resist mask 190R can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is positioned on the outermost surface and the organic compound film 103Rf is not exposed; thus, the organic compound film 103Rf can be inhibited from being damaged in the step of removing the resist mask 190R. In addition, the range of choices for the method for removing the resist mask 190R can be widened.

Next, as illustrated in FIG. 6D, the organic compound film 103Rf is processed to form the organic compound layer 103R. For example, part of the organic compound film 103Rf is removed using the mask layer 159R and the sacrificial layer 158R as a hard mask to form the organic compound layer 103R.

Accordingly, as illustrated in FIG. 6D, a stacked-layer structure of the organic compound layer 103R, the sacrificial layer 158R, and the mask layer 159R remains over the conductive layer 152R. The conductive layer 152G and the conductive layer 152B are exposed.

FIG. 6D illustrates an example where the end portion of the organic compound layer 103R is positioned outward from the end portion of the conductive layer 152R. Such a structure can increase the aperture ratio of the pixel. Although not illustrated in FIG. 6D, by the above etching treatment, a depressed portion may be formed in the insulating layer 175 in a region not overlapping with the organic compound layer 103R.

The organic compound layer 103R covers the top surface and the side surface of the conductive layer 152R and thus, the subsequent steps can be performed without exposure of the conductive layer 152R. When the end portion of the conductive layer 152R is exposed, corrosion might occur in the etching step, for example. A product generated by corrosion of the conductive layer 152R may be unstable, and for example, might be dissolved in a solution when wet etching is performed and might be scattered in an atmosphere when dry etching is performed. The product dissolved in a solution or scattered in an atmosphere might be attached to a surface to be processed, the side surface of the organic compound layer 103R, and the like, which adversely affects the characteristics of the light-emitting device or forms a leakage path between the light-emitting devices in some cases. In a region where the end portion of the conductive layer 152R is exposed, adhesion between layers in contact with each other might be lowered, which might be likely to cause peeling of the organic compound layer 103R or the conductive layer 152R.

Thus, the structure where the organic compound layer 103R covers the top surface and the side surface of the conductive layer 152R can improve the yield and characteristics of the light-emitting device, for example.

As described above, the resist mask 190R is preferably provided to cover the area from the end portion of the organic compound layer 103R to the end portion of the conductive layer 152C (the end portion on the organic compound layer 103R side) in the cross section B1-B2. Thus, as illustrated in FIG. 6D, the sacrificial layer 158R and the mask layer 159R are provided to cover the area from the end portion of the organic compound layer 103R to the end portion of the conductive layer 152C (the end portion on the organic compound layer 103R side) in the cross section B1-B2. Hence, the insulating layer 175 can be inhibited from being exposed in the cross section B1-B2, for example. This can prevent the insulating layer 175, the insulating layer 174, and the insulating layer 173 from being partly removed by etching and thus prevent the conductive layer 179 from being exposed. Thus, unintentional electrical connection between the conductive layer 179 and another conductive layer can be inhibited. For example, a short circuit between the conductive layer 179 and a common electrode 155 formed in a later step can be inhibited.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Thus, the etching can be performed under a low-power condition while an adequately high etching rate is maintained. Thus, damage to the organic compound film 103Rf can be inhibited. Furthermore, a defect such as attachment of a reaction product generated in the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He or Ar as the etching gas, for example. Alternatively, a gas containing oxygen and at least one kind of the above is preferably used as the etching gas. Alternatively, an oxygen gas may be used as the etching gas. Specifically, for example, a gas containing H₂ and Ar or a gas containing CF₄ and He can be used as the etching gas. For example, a gas containing CF₄, He, and oxygen can be used as the etching gas. For example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf, and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a lithography method. Note that part of the organic compound film 103Rf may be removed using the resist mask 190R. Then, the resist mask 190R may be removed.

Next, hydrophobic treatment for the conductive layer 152G, for example, is preferably performed. At the time of processing the organic compound film 103Rf, the surface of the conductive layer 152G changes to have hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling. Note that the hydrophobization treatment is not necessarily performed.

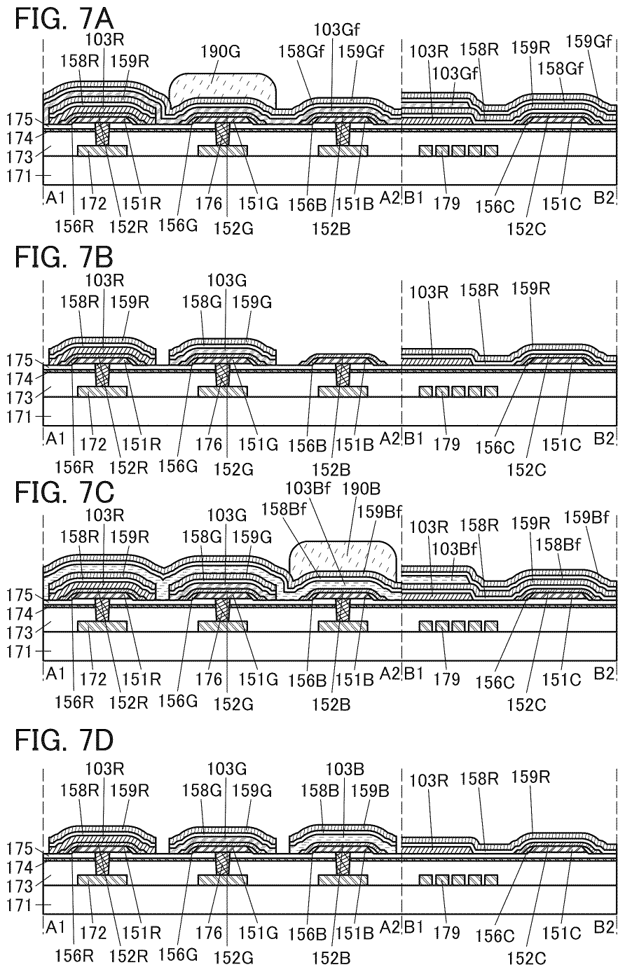

Next, as illustrated in FIG. 7A, an organic compound film 103Gf to be the organic compound layer 103G later is formed over the conductive layer 152G, the conductive layer 152B, the mask layer 159R, and the insulating layer 175.

The organic compound film 103Gf can be formed by a method similar to that for forming the organic compound film 103Rf The organic compound film 103Gf can have a structure similar to that of the organic compound film 103Rf.

Then, as illustrated in FIG. 7A, a sacrificial film 158Gf to be a sacrificial layer 158G later and a mask film 159Gf to be a mask layer 159G later are sequentially formed over the organic compound film 103Gf and the mask layer 159R. After that, a resist mask 190G is formed. The materials and the formation methods of the sacrificial film 158Gf and the mask film 159Gf are similar to conditions applicable to the sacrificial film 158Rf and the mask film 159Rf The materials and the formation method of the resist mask 190G are similar to conditions applicable to the resist mask 190R.

The resist mask 190G is provided at a position overlapping with the conductive layer 152G.

Subsequently, as illustrated in FIG. 7B, part of the mask film 159Gf is removed using the resist mask 190G, whereby the mask layer 159G is formed. The mask layer 159G remains over the conductive layer 152G. After that, the resist mask 190G is removed. Next, part of the sacrificial film 158Gf is removed using the mask layer 159G as a mask, whereby the sacrificial layer 158G is formed. Next, the organic compound film 103Gf is processed to form the organic compound layer 103G. For example, part of the organic compound film 103Gf is removed using the mask layer 159G and the sacrificial layer 158G as a hard mask, whereby the organic compound film 103G is formed.

Accordingly, as illustrated in FIG. 7B, the stacked-layer structure of the organic compound layer 103G, the sacrificial layer 158G, and the mask layer 159G remains over the conductive layer 152G. The mask layer 159R and the conductive layer 152B are exposed.

Next, hydrophobic treatment for the conductive layer 152B, for example, is preferably performed. At the time of processing the organic compound film 103Gf, the surface of the conductive layer 152B changes to have hydrophilic properties in some cases, for example. The hydrophobic treatment for the conductive layer 152B, for example, can increase the adhesion between the conductive layer 152B and a layer to be formed in a later step (which is the organic compound layer 103B here) and inhibit film peeling. Note that the hydrophobization treatment is not necessarily performed.

Next, as illustrated in FIG. 7C, an organic compound film 103Bf to be the organic compound layer 103B later is formed over the conductive layer 152B, the mask layer 159R, the mask layer 159G, and the insulating layer 175.

The organic compound film 103Bf can be formed by a method similar to that for forming the organic compound film 103Rf The organic compound film 103Bf can have a structure similar to that of the organic compound film 103Rf.

Then, as illustrated in FIG. 7C, a sacrificial film 158Bf to be a sacrificial layer 158B later and a mask film 159Bf to be a mask layer 159B later are sequentially formed over the organic compound film 103Bf and the mask layer 159R. After that, a resist mask 190B is formed. The materials and the formation methods of the sacrificial film 158Bf and the mask film 159Bf are similar to conditions applicable to the sacrificial film 158Rf and the mask film 159Rf The materials and the formation method of the resist mask 190B are similar to conditions applicable to the resist mask 190R.

The resist mask 190B is provided at a position overlapping with the conductive layer 152B.

Subsequently, as illustrated in FIG. 7D, part of the mask film 159Bf is removed using the resist mask 190B, whereby the mask layer 159B is formed. The mask layer 159B remains over the conductive layer 152B. After that, the resist mask 190B is removed. Then, part of the sacrificial film 158Bf is removed using the mask layer 159B as a mask, whereby the sacrificial layer 158B is formed. Next, the organic compound film 103Bf is processed to form the organic compound layer 103B. For example, part of the organic compound film 103Bf is removed using the mask layer 159B and the sacrificial layer 158B as a hard mask, whereby the organic compound layer 103B is formed.

Accordingly, as illustrated in FIG. 7D, a stacked-layer structure of the organic compound layer 103B, the sacrificial layer 158B, and the mask layer 159B remains over the conductive layer 152B. The mask layer 159R and the mask layer 159G are exposed.

Note that the side surfaces of the organic compound layer 103R, the organic compound layer 103G, and the organic compound layer 103B are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layer 103R, the organic compound layer 103G, and the organic compound layer 103B, which are formed by a photolithography method as described above, can be shortened to less than or equal to 8 μm, less than or equal to 5 μm, less than or equal to 3 μm, less than or equal to 2 μm, or less than or equal to 1 μm. Here, the distance can be specified, for example, by a distance between facing end portions of two adjacent layers among the organic compound layer 103R, the organic compound layer 103G, and the organic compound layer 103B. The distance between the island-shaped organic compound layers is shortened in this manner, whereby a display apparatus with high resolution and a high aperture ratio can be provided. In addition, the distance between the first electrodes of adjacent light-emitting devices can also be shortened to, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

Next, the mask layer 159R, the mask layer 159G, and the mask layer 159B are preferably removed as illustrated in FIG. 8A. The sacrificial layer 158R, the sacrificial layer 158G, the sacrificial layer 158B, the mask layer 159R, the mask layer 159G, and the mask layer 159B might remain in the display apparatus in some cases depending on the subsequent steps. Removing the mask layer 159R, the mask layer 159G, and the mask layer 159B at this stage can inhibit the mask layer 159R, the mask layer 159G, and the mask layer 159B from remaining in the display apparatus. In the case where a conductive material is used for the mask layer 159R, the mask layer 159G, and the mask layer 159B, removing the mask layer 159R, the mask layer 159G, and the mask layer 159B in advance can inhibit generation of leakage current, formation of a capacitor, and the like due to the mask layer 159R, the mask layer 159G, and the mask layer 159B, for example.

This embodiment describes an example where the mask layer 159R, the mask layer 159G, and the mask layer 159B are removed; however, the mask layer 159R, the mask layer 159G, and the mask layer 159B are not necessarily removed. For example, in the case where the mask layer 159R, the mask layer 159G, and the mask layer 159B contain the material having a property of blocking ultraviolet rays, the procedure preferably proceeds to the next step without removing the mask layer 159R, the mask layer 159G, and the mask layer 159B, in which case the organic compound layer can be protected from ultraviolet rays.

The step of removing the mask layers can be performed by a method similar to that for the step of processing the mask layers. Specifically, by using a wet etching method, damage applied to the organic compound layer 103R, the organic compound layer 103G, and the organic compound layer 103B at the time of removing the mask layers can be reduced as compared with the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layer 103R, the organic compound layer 103G, and the organic compound layer 103B and water adsorbed on the surfaces of the organic compound layer 103R, the organic compound layer 103G, and the organic compound layer 103B. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 8B, the inorganic insulating film 125f to be the inorganic insulating layer 125 later is formed to cover the organic compound layer 103R, the organic compound layer 103G, the organic compound layer 103B, the sacrificial layer 158R, the sacrificial layer 158G, and the sacrificial layer 158B.

As described later, an insulating film to be the insulating layer 127 later is formed in contact with the top surface of the inorganic insulating film 125f. Therefore, the top surface of the inorganic insulating film 125f preferably has high affinity for a material used for the insulating film (e.g., a photosensitive resin composition containing an acrylic resin). To improve the affinity, surface treatment is preferably performed so that the top surface of the inorganic insulating film 125f is made hydrophobic (or its hydrophobic properties are improved). For example, it is preferable to perform the treatment using a silylation agent such as hexamethyldisilazane (HMIDS). By making the top surface of the inorganic insulating film 125f hydrophobic in such a manner, an insulating film 127f can be formed with favorable adhesion. Note that the above-described hydrophobic treatment may be performed as the surface treatment.

Then, as illustrated in FIG. 8C, the insulating film 127f to be the insulating layer 127 later is formed over the inorganic insulating film 125f.

The inorganic insulating film 125f and the insulating film 127f are preferably formed by a formation method that causes less damage to the organic compound layer 103R, the organic compound layer 103G, and the organic compound layer 103B. The inorganic insulating film 125f, which is formed in contact with the side surfaces of the organic compound layer 103R, the organic compound layer 103G, and the organic compound layer 103B, is particularly preferably formed by a method that causes less damage to the organic compound layer 103R, the organic compound layer 103G, and the organic compound layer 103B than the method for forming the insulating film 127f.

The inorganic insulating film 125f and the insulating film 127f are each formed at a temperature lower than the upper temperature limit of the organic compound layer 103R, the organic compound layer 103G, and the organic compound layer 103B. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case deposition damage can be reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher film formation rate than an ALD method. In that case, a highly reliable display apparatus can be manufactured with high productivity.

The insulating film 127f is preferably formed by the aforementioned wet film formation method. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limit of the organic compound layer 103R, the organic compound layer 103G, and the organic compound layer 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent contained in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are interposed between any two of the conductive layer 152R, the conductive layer 152G, and the conductive layer 152B and around the conductive layer 152C. Thus, the conductive layer 152R, the conductive layer 152G, the conductive layer 152B, and the conductive layer 152C are irradiated with visible light or ultraviolet rays from above. Note that when a negative photosensitive material is used for the insulating film 127f, the region where the insulating layer 127 is to be formed is irradiated with visible light or ultraviolet rays.

The width of the insulating layer 127 to be formed later can be controlled in accordance with the exposed region of the insulating film 127f. In this embodiment, processing is performed such that the insulating layer 127 includes a portion overlapping with the top surface of the conductive layer 151.

Light used for the light exposure preferably includes the i-line (wavelength: 365 nm). The light used for the light exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layer 158R, the sacrificial layer 158G, and the sacrificial layer 158B) and the inorganic insulating film 125f, diffusion of oxygen into the organic compound layer 103R, the organic compound layer 103G, and the organic compound layer 103B can be inhibited. When the organic compound layer is irradiated with light (visible light or ultraviolet rays), an organic compound included in the organic compound layer is brought into an excited state and a reaction between the organic compound and oxygen contained in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layer 158 and the inorganic insulating film 125f over the island-shaped organic compound layer, bonding of oxygen in the atmosphere to the organic compound included in the organic compound layer can be inhibited.

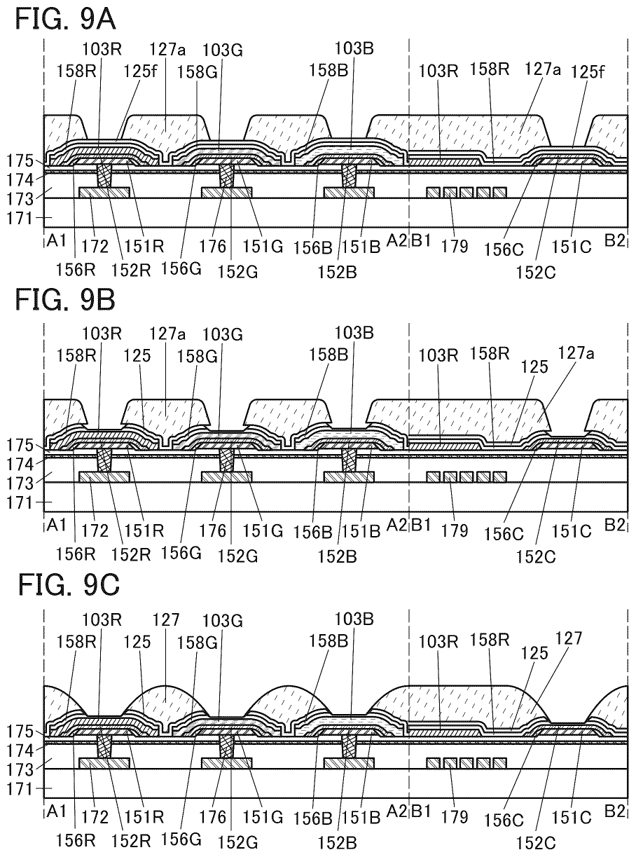

Next, as illustrated in FIG. 9A, development is performed to remove the exposed region of the insulating film 127f, whereby an insulating layer 127a is formed. The insulating layer 127a is formed in regions that are interposed between any two of the conductive layer 152R, the conductive layer 152G, and the conductive layer 152B and a region surrounding the conductive layer 152C. Here, when an acrylic resin is used for the insulating film 127f, an alkaline solution, e.g., TMAH, can be used as a developer.

Then, a residue (what is called scum) due to the development may be removed. For example, the residue can be removed by ashing using oxygen plasma.

Etching may be performed to adjust the surface level of the insulating layer 127a. The insulating layer 127a may be processed by ashing using oxygen plasma, for example. In the case where a non-photosensitive material is used for the insulating film 127f, the surface level of the insulating film 127f can be adjusted by the ashing, for example.

Next, as illustrated in FIG. 9B, etching treatment is performed using the insulating layer 127a as a mask to remove part of the inorganic insulating film 125f and reduce the thickness of part of the sacrificial layer 158R, the sacrificial layer 158G, and the sacrificial layer 158B. Thus, the inorganic insulating layer 125 is formed under the insulating layer 127a. Moreover, the surfaces of the thin portions of the sacrificial layer 158R, the sacrificial layer 158G, and the sacrificial layer 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layer 158R, the sacrificial layer 158G, and the sacrificial layer 158B, in which case the first etching treatment can be performed collectively.

By etching using the insulating layer 127a with a tapered side surface as a mask, the side surface of the inorganic insulating layer 125 and upper end portions of the side surfaces of the sacrificial layer 158R, the sacrificial layer 158G, and the sacrificial layer 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layer 158R, the sacrificial layer 158G, and the sacrificial layer 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure where a high-frequency voltage is applied to one of the parallel plate electrodes. Alternatively, a structure may be employed where different high-frequency voltages are applied to one of the parallel plate electrodes. Alternatively, a structure may be employed where high-frequency voltages with the same frequency are applied to the parallel plate electrodes. Alternatively, a structure may be employed where high-frequency voltages with different frequencies are applied to the parallel plate electrodes.

In the case of performing dry etching, a by-product or the like generated by the dry etching might be deposited on the top surface and the side surface of the insulating layer 127a, for example. Thus, a component contained in the etching gas, a component contained in the inorganic insulating film 125f, components contained in the sacrificial layer 158R, the sacrificial layer 158G, and the sacrificial layer 158B, or the like might be contained in the insulating layer 127 after the display apparatus is completed.

The first etching treatment is preferably performed by wet etching. The use of wet etching can reduce damage to the organic compound layer 103R, the organic compound layer 103G, and the organic compound layer 103B, as compared with the case of using dry etching. Wet etching can be performed using an alkaline solution, for example. For example, TMAH, which is an alkaline solution, can be used for the wet etching of an aluminum oxide film. In that case, puddle wet etching can be performed. Note that the inorganic insulating film 125f is preferably formed using a material similar to that of the sacrificial layer 158R, the sacrificial layer 158G, and the sacrificial layer 158B, in which case the etching treatment can be performed collectively.

In the first etching treatment, the etching treatment is stopped when the sacrificial layer 158R, the sacrificial layer 158G, and the sacrificial layer 158B are thinned before the sacrificial layers are completely removed. The sacrificial layer 158R, the sacrificial layer 158G, and the sacrificial layer 158B remain respectively over the organic compound layer 103R, the organic compound layer 103G, and the organic compound layer 103B in this manner, whereby the organic compound layer 103R, the organic compound layer 103G, and the organic compound layer 103B can be prevented from being damaged by treatment in a later step.

Next, light exposure is preferably performed on the entire substrate so that the insulating layer 127a is irradiated with visible light or ultraviolet rays. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a into a tapered shape.

Here, when a barrier insulating layer against oxygen (e.g., an aluminum oxide film) is provided as each of the sacrificial layer 158R, the sacrificial layer 158G, and the sacrificial layer 158B, diffusion of oxygen into the organic compound layer 103R, the organic compound layer 103G, and the organic compound layer 103B can be inhibited. When the organic compound layer is irradiated with light (visible light or ultraviolet rays), an organic compound included in the organic compound layer is brought into an excited state and a reaction with oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layer 158R, the sacrificial layer 158G, and the sacrificial layer 158B over the island-shaped organic compound layer, bonding of oxygen in the atmosphere to the organic compound contained in the organic compound layer can be inhibited.

After that, heat treatment (also referred to as post-baking) is performed. The heat treatment can change the insulating layer 127a into the insulating layer 127 having a tapered side surface (see FIG. 9C). The heat treatment is performed at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be either an air atmosphere or an inert gas atmosphere. Alternatively, the heating atmosphere may be either an atmospheric pressure atmosphere or a reduced-pressure atmosphere. The heat treatment in this step is preferably performed at a higher substrate temperature than the heat treatment (pre-baking) after formation of the insulating film 127f. Accordingly, adhesion between the insulating layer 127 and the inorganic insulating layer 125 can be improved, and corrosion resistance of the insulating layer 127 can be increased.

When the sacrificial layer 158R, the sacrificial layer 158G, and the sacrificial layer 158B are not completely removed by the first etching treatment and the sacrificial layer 158R, the sacrificial layer 158G, and the sacrificial layer 158B with reduced thicknesses remain, the organic compound layer 103R, the organic compound layer 103G, and the organic compound layer 103B can be prevented from being damaged and deteriorating in the heat treatment. This increases the reliability of the light-emitting device.

The side surface of the insulating layer 127 might have a concave shape depending on the material of the insulating layer 127, and the temperature, time, and atmosphere of the post-baking. For example, the insulating layer 127 is more likely to be changed in shape to have a concave shape as the post-baking is performed at higher temperature or for a longer time.

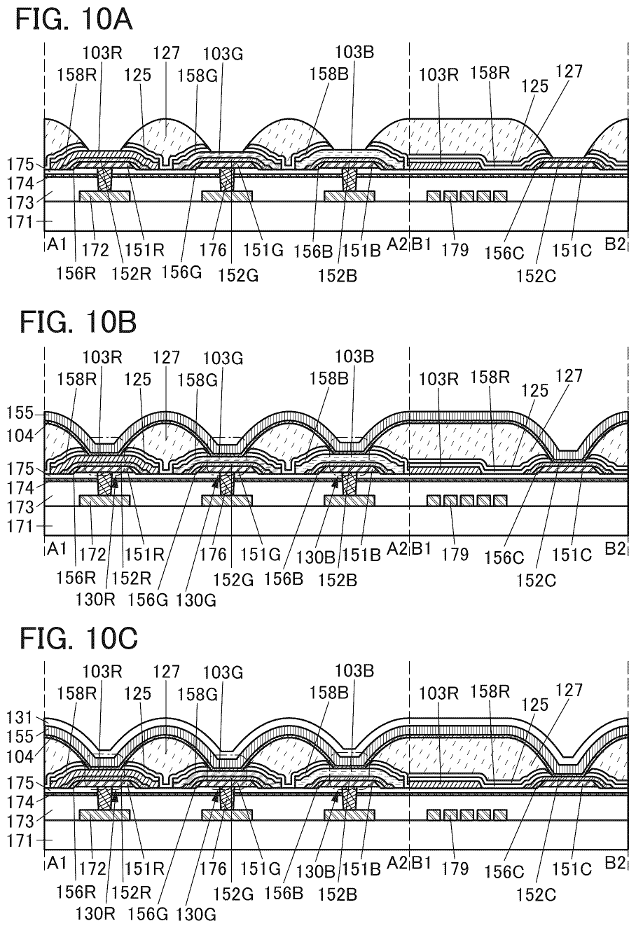

Next, as illustrated in FIG. 10A, etching treatment is performed using the insulating layer 127 as a mask to remove parts of the sacrificial layer 158R, the sacrificial layer 158G, and the sacrificial layer 158B. Note that part of the inorganic insulating layer 125 is also removed in some cases. Thus, openings are formed in the sacrificial layer 158R, the sacrificial layer 158G, and the sacrificial layer 158B, and the top surfaces of the organic compound layer 103R, the organic compound layer 103G, the organic compound layer 103B, and the conductive layer 152C are exposed. Note that the etching treatment using the insulating layer 127 as a mask may be hereinafter referred to as second etching treatment.

The end portion of the inorganic insulating layer 125 is covered with the insulating layer 127. FIG. 10A illustrates an example where part of an end portion of the sacrificial layer 158G (specifically, a tapered portion formed by the first etching treatment) is covered with the insulating layer 127 and the tapered portion formed by the second etching treatment is exposed.

If the first etching treatment is not performed and the inorganic insulating layer 125 and the mask layer are collectively etched after the post-baking, the inorganic insulating layer 125 and the mask layer under the end portion of the insulating layer 127 may disappear because of side etching and a cavity may be formed. The cavity causes unevenness of the surface where the common electrode 155 is formed, so that step disconnection is likely to occur in the common electrode 155. Even when a cavity is formed due to side etching of the inorganic insulating layer 125 and the mask layer by the first etching treatment, the post-baking performed subsequently can make the insulating layer 127 fill the cavity. After that, the thinned mask layer is etched by the second etching treatment; thus, the amount of side etching decreases, a cavity is less likely to be formed, and even if a cavity is formed, it can be extremely small. Therefore, the surface where the common electrode 155 is formed can be flatter.

Note that the insulating layer 127 may cover the entire end portion of the sacrificial layer 158G. For example, the end portion of the insulating layer 127 may droop to cover the end portion of the sacrificial layer 158G. As another example, the end portion of the insulating layer 127 may be in contact with the top surface of at least one of the organic compound layer 103R, the organic compound layer 103G, and the organic compound layer 103B. As described above, in the case where light exposure is not performed on the insulating layer 127a after development, the shape of the insulating layer 127 is likely to change in some cases.

The second etching treatment is performed by wet etching. The use of wet etching can reduce damage to the organic compound layer 103R, the organic compound layer 103G, and the organic compound layer 103B, as compared with the case of using dry etching. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layer 151 and the conductive layer 152 that has a lower spontaneous potential than the other suffers from galvanic corrosion in some cases. For example, when the conductive layer 151 is formed using aluminum and the conductive layer 152 is formed using an indium tin oxide, the conductive layer 152 sometimes corrodes. This might decrease the yield of the display apparatus. In addition, the reliability of the display apparatus is decreased in some cases.

The conductive layer 152 is formed to cover the top surface and the side surface of the conductive layer 151 as described above; thus, even when gaps exist at the interface between the organic compound layer 103 and the sacrificial layer 158, the interface between the organic compound layer 103 and the inorganic insulating layer 125, and the interface between the organic compound layer 103 and the insulating layer 175, the chemical solution can be prevented from coming into contact with the conductive layer 151 in the second etching treatment. Thus, corrosion of the pixel electrode, e.g., the conductive layer 152, can be prevented.

Furthermore, when the insulating layer 156 is formed to include a region overlapping with the side surface of the conductive layer 151 and the conductive layer 152 is formed to cover the conductive layer 151 and the insulating layer 156, step disconnection of the conductive layer 152 can be prevented, whereby the chemical solution can be prevented from coming into contact with the conductive layer 151 in the second etching treatment, for example. Thus, corrosion of the pixel electrode, e.g., the conductive layer 152, can be prevented.

As described above, by providing the insulating layer 127, the inorganic insulating layer 125, the sacrificial layer 158R, the sacrificial layer 158G, and the sacrificial layer 158B, a connection defect due to a disconnected portion and an increase in electrical resistance due to a locally thinned portion can be inhibited from occurring in the common electrode 155 between the light-emitting devices. Thus, the display quality of the display apparatus of one embodiment of the present invention can be improved.

Heat treatment is further performed after parts of the organic compound layer 103R, the organic compound layer 103G, and the organic compound layer 103B are exposed. The heat treatment can remove water contained in the organic compound layers, water adsorbed onto the surfaces of the organic compound layers, and the like. The shape of the insulating layer 127 may be changed by the heat treatment. Specifically, the insulating layer 127 may be widened to cover at least one of the end portion of the inorganic insulating layer 125, the end portions of the sacrificial layer 158R, the sacrificial layer 158G, and the sacrificial layer 158B, and the top surfaces of the organic compound layer 103R, the organic compound layer 103G, and the organic compound layer 103B.

When the temperature of the heat treatment is too low, water contained in the organic compound layers, water adsorbed onto the surfaces of the organic compound layers, and the like cannot be sufficiently removed. When the temperature of the heat treatment is too high, the organic compound layer 103 might deteriorate and the insulating layer 127 might change in shape excessively. Thus, the temperature of the heat treatment is preferably higher than the temperature at which water is released from the organic compound layer 103 and lower than the T_{g} of the organic compound included in the organic compound layer 103, further preferably lower than the T_{g} of the organic compound included in the top surface of the organic compound layer 103. Specifically, the heat treatment is preferably performed at a substrate temperature higher than or equal to 80° C. and lower than or equal to 130° C., preferably higher than or equal to 90° C. and lower than or equal to 120° C., further preferably higher than or equal to 100° C. and lower than or equal to 120° C., still further preferably higher than or equal to 100° C. and lower than or equal to 110° C. The heating atmosphere may be either an air atmosphere or an inert gas atmosphere. Although the heating atmosphere may be an atmospheric-pressure atmosphere or a reduced-pressure atmosphere, a reduced-pressure atmosphere is preferably employed to prevent re-adsorption of water released from the organic compound layer 103.

By the heat treatment, water contained in the organic compound layer 103R, the organic compound layer 103G, and the organic compound layer 103B, water adsorbed onto the surfaces of the organic compound layers, and the like can be sufficiently removed without causing deterioration of the organic compound layers and an excessive change in the shape of the insulating layer 127. Thus, degradation of the characteristics of the light-emitting devices can be prevented.

Then, as illustrated in FIG. 10B, the common layer 104 and the common electrode 155 are formed over the organic compound layer 103R, the organic compound layer 103G, the organic compound layer 103B, the conductive layer 152C, and the insulating layer 127. The common layer 104 and the common electrode 155 can be formed by a method such as a sputtering method or a vacuum evaporation method. The common layer 104 may be formed by an evaporation method and the common electrode 155 may be formed by a sputtering method.

Next, as illustrated in FIG. 10C, the protective layer 131 is formed over the common electrode 155. The protective layer 131 can be formed by a vacuum evaporation method, a sputtering method, a CVD method, an ALD method, or the like.

Subsequently, the substrate 120 is attached onto the protective layer 131 with the resin layer 122, whereby the display apparatus can be manufactured. In the method for manufacturing the display apparatus of one embodiment of the present invention, the insulating layer 156 is provided to include a region overlapping with the side surface of the conductive layer 151, and the conductive layer 152 is formed to cover the conductive layer 151 and the insulating layer 156 as described above. This can increase the yield of the display apparatus and inhibit generation of a defect.

As described above, in the method for manufacturing a display apparatus of one embodiment of the present invention, the island-shaped organic compound layer 103R, the island-shaped organic compound layer 103G, and the island-shaped organic compound layer 103B are formed not by using a fine metal mask but by processing a film formed over the entire surface; thus, the island-shaped layers can be formed to have a uniform thickness. Accordingly, a display apparatus having high resolution or a display apparatus having a high aperture ratio can be achieved. Furthermore, even when the resolution or the aperture ratio is high and the distance between the subpixels is extremely short, the organic compound layer 103R, the organic compound layer 103G, and the organic compound layer 103B can be inhibited from being in contact with each other in the adjacent subpixels. As a result, generation of a leakage current between the subpixels can be inhibited. This can prevent crosstalk, so that the display apparatus can achieve extremely high contrast. Furthermore, even when a display apparatus includes a tandem light-emitting device formed by a lithography method, the display apparatus can have favorable characteristics.

### Embodiment 4

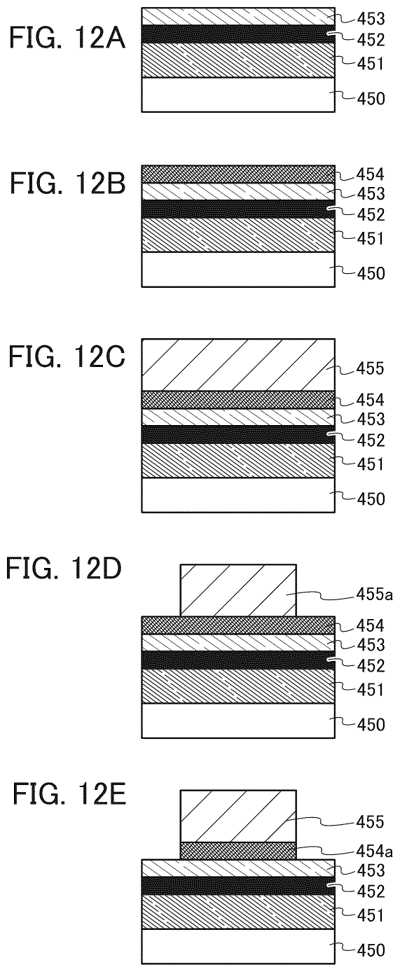

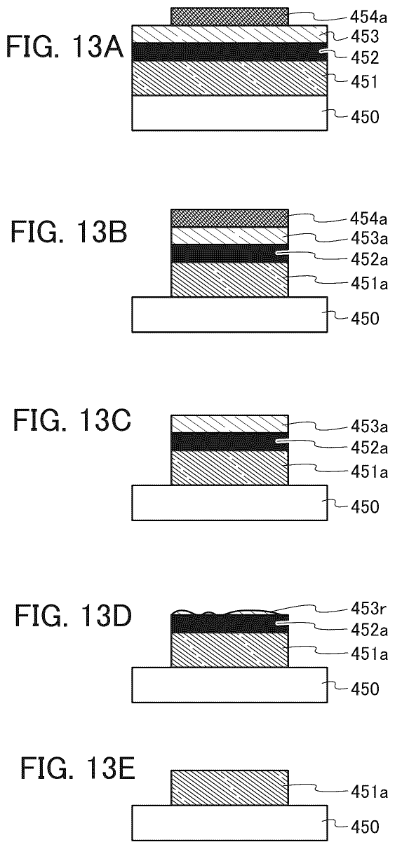

In this embodiment, a method for processing an organic compound film with the use of the organic compound of one embodiment of the present invention for an organic mask film is described with reference to FIG. 12 and FIG. 13.

The shape processing of an organic compound film by a lithography method requires many problems to be overcome. Examples of these problems include the influence of exposure of the organic compound film to the air, the influence of light irradiation at the time of exposure of a photosensitive resin to light, the influence of a developer to which the light-exposed photosensitive resin is exposed at the time of development, and the influence of formation of a metal film that is sometimes formed to reduce the influence of the developer.

These influences are considered problematic because they eliminate the organic compound film itself, or damage a surface of the organic compound film to significantly degrade the characteristics of a device to be manufactured later, for example.

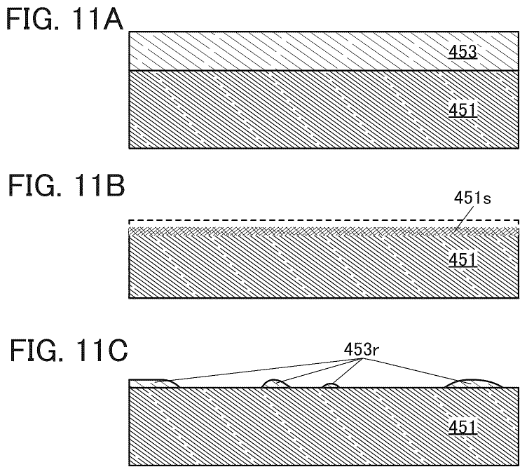

Here, as one means to solve the above problems, an inorganic mask film 453 is provided over and in contact with an organic compound film 451 as a protective film as in FIG. 11A, and then a step that would cause the above-described problems is performed. As the inorganic mask film, for example, an aluminum oxide film is used. Since an aluminum oxide film can be formed dense and is highly capable of blocking liquid and gas, the adverse effects caused by the step can be inhibited. Furthermore, the aluminum oxide film can be formed and removed by a method that causes less damage to the organic compound film, and thus is extremely suitable as the inorganic mask film 453 protecting the organic compound film 451.

Note that an atomic layer deposition (ALD) method that is capable of forming a denser film and causes less damage to the organic compound film is preferable as a formation method of the aluminum oxide film.

As described above, since the formation and removal of an aluminum oxide film cause relatively less damage to the organic semiconductor film, an aluminum oxide film can be suitably used as a protective film in processing of the organic compound film by a lithography method. However, it is natural that a surface 451s of the organic compound film 451 is damaged, as illustrated in FIG. 11B, when the surface of the organic compound film is excessively exposed to a step of removing the aluminum oxide film, whereby the characteristics of the organic semiconductor might be degraded. Accordingly, the time taken for the removal of the aluminum oxide film is preferably as short as possible.

In order to minimize the time of the removal step, the treatment is preferably ended once the inorganic mask film 453 is removed from an upper surface of the organic compound film. However, it is extremely difficult to determine whether the inorganic mask film 453 is removed from the upper surface of the organic compound film. In the case where the inorganic mask film 453 has in-plane variation in the film quality, in-plane variation is also caused in the etching rate in etching that is the removal step of the inorganic mask film 453; accordingly, even when some portions of the inorganic mask film can be removed, inorganic mask residues 453r might remain in the other portions, as illustrated in FIG. 11C. In particular, in the case where an aluminum oxide film is provided as the inorganic mask film 453 over the organic compound film 451 by an ALD method, the film formation cannot be performed at high temperature; thus, the above-described in-plane variation is easily caused and accordingly the inorganic mask residues 453r, which remain due to the in-plane variation, might be generated in some portions. When the inorganic mask film remains over the organic compound film, the driving voltage of a device manufactured later might increase. Note that excessive etching for removing all of the inorganic mask residues 453r is extremely undesirable because such etching might cause side etching of the inorganic mask film that should remain in the process and is provided in an adjacent pixel direction (unremoved inorganic mask film) from the lateral direction.

Thus, in one embodiment of the present invention, an organic mask film 452 is provided between the organic compound film 451 and the inorganic mask film 453 to facilitate the removal of the aluminum oxide film. For the organic mask film 452, the organic compound of one embodiment of the present invention can be used. Note that for the organic mask film 452, an organic compound having high water solubility is further preferably used. As described above, the organic compound of one embodiment of the present invention sometimes has increased water solubility depending on the number of ring members of the bicyclic guanidine skeleton. The organic compound of one embodiment of the present invention having such increased water solubility is further preferably used for the organic mask film 452.

First, the organic compound film 451 is formed over a base film 450 (FIG. 12A). The base film may be either an insulating film or a conductive film depending on a device manufactured later. The organic compound film 451 may be formed by a dry process such as an evaporation method or a wet process such as a spin coating method.

Next, the organic mask film 452 including the organic compound of one embodiment of the present invention described in Embodiment 1 is formed over the organic compound film 451 (FIG. 12A). The organic mask film 452 is preferably formed by a vacuum evaporation method.

Next, the inorganic mask film 453 is formed over the organic mask film 452 (FIG. 12A). The inorganic mask film 453 is preferably formed by a method that causes less damage to the film in contact with the organic compound film 451.

A film 454 to be a hard mask formed of a metal film or a metal compound film is preferably formed over the inorganic mask film 453 (FIG. 12B). Since the inorganic mask film 453 can reduce damage to the organic compound film 451, a film formation method that causes relatively great damage to a formation surface, such as a sputtering method, can be selected for the formation of the film 454 to be a hard mask.

Examples of a material forming the film 454 to be a hard mask include silicon, silicon nitride, silicon oxide, tungsten, titanium, molybdenum, tantalum, tantalum nitride, an alloy containing molybdenum and niobium, or an alloy metal oxide containing molybdenum and tungsten, and a metal oxide such as an indium gallium zinc oxide (also denoted as In—Ga—Zn oxide or IGZO). It is also possible to use indium oxide, an indium zinc oxide (In—Zn oxide), an indium tin oxide (In—Sn oxide), an indium titanium oxide (In—Ti oxide), an indium tin zinc oxide (In—Sn—Zn oxide), an indium titanium zinc oxide (In—Ti—Zn oxide), an indium gallium tin zinc oxide (In—Ga—Sn—Zn oxide), or the like. Alternatively, an indium tin oxide containing silicon can also be used, for example.

Then, a photosensitive resin is applied to the film 454 to be a hard mask, so that a resin film 455 is formed. The photosensitive resin may be either a positive type resist or a negative type resist.

Next, a photomask layer 455a is formed by performing light exposure in accordance with the photosensitivity of the resin and performing development (FIG. 12D), and the film 454 to be a hard mask is etched using the photomask layer 455a, whereby a hard mask layer 454a is formed (FIG. 12E).

The film 454 to be a hard mask may be etched by either wet etching or dry etching. This etching is preferably performed under the condition where the film 454 to be a hard mask has a high selectivity with respect to the inorganic mask film 453.

After the hard mask layer 454a is formed, the photomask layer 455a is removed (FIG. 13A). Owing to the film 454 to be a hard mask and the inorganic mask film 453, the organic compound film 451 is prevented from being adversely affected, for example, being eliminated or being damaged, by treatment in forming and removing the photomask layer 455a; thus, an organic semiconductor device having favorable characteristics can be manufactured.

After that, etching is performed using the film 454 to be a hard mask as a mask, whereby an organic compound layer 451a, an organic mask layer 452a, and an inorganic mask layer 453a are formed (FIG. 13B). This etching may be either wet etching or dry etching, but dry etching is preferable.

After processing the organic compound layer 451a is completed, the hard mask layer 454a is removed (FIG. 13C). The hard mask layer 454a is removed by etching. Although either wet etching or dry etching can be employed, dry etching is preferable.. This etching is preferably performed under the condition where the hard mask layer 454a has a high selectivity with respect to the inorganic mask layer 453a.

Lastly, the inorganic mask layer 453a and the organic mask layer 452a are treated with water or a liquid containing water as a solvent to be removed at once (FIG. 13E).

As the removing method, the inorganic mask layer 453a and the organic mask layer 452a are immersed in water or the liquid containing water as a solvent for a predetermined time, and then showered with pure water. The hard mask layer 454a and the organic mask layer 452a can be removed by just performing this step when the organic mask film 452 is formed using an organic compound having high water solubility. The liquid used for the removal is preferably water because water causes less damage to the organic semiconductor layer 451a.

Note that after the hard mask layer 454a is removed, the inorganic mask layer 453a may be removed to some extent before the organic mask layer 452a is treated with water or the liquid containing water as a solvent (FIG. 13D). The inorganic mask layer 453a is removed by etching. Although either wet etching or dry etching can be employed, wet etching using an alkaline solution or an acid solution is preferable, and wet etching using an alkaline solution is further preferable. The surface of the organic compound layer 451a is prevented from being exposed to the alkaline solution or the acid solution owing to the organic mask layer 452a, whereby degradation of the characteristics can be prevented. Furthermore, treatment is performed such that inorganic mask residues 453r remain over the organic mask layer 452a to some extent, whereby the next step of removing the organic mask layer 452a can be performed more smoothly.

Since the organic mask layer 452a uses the organic compound film of one embodiment of the present invention having an electron-injection property, the organic mask film 452 is not necessarily removed completely and may remain partially or entirely.

The organic compound layer 451a processed through the above steps has less processing damage, so that an organic device can have favorable characteristics. Furthermore, the inorganic mask residues 453r can be inhibited from remaining on the surface of the organic compound layer 451a, whereby an increase in voltage of an organic device manufactured later can be prevented.

The structure of this embodiment can be used in combination with any of the structures of the other embodiments as appropriate.

### Embodiment 5

In this embodiment, display apparatuses of embodiments of the present invention will be described.

The display apparatus of this embodiment can be a high-resolution display apparatus. Accordingly, the display apparatus of this embodiment can be used for display portions of information terminals (wearable devices) such as watch-type and bracelet-type information terminals and display portions of wearable devices capable of being worn on a head, such as a VR device like a head-mounted display (HMD) and a glasses-type AR device.

The display apparatus of this embodiment can be a display apparatus having high definition or a large-sized display apparatus. Accordingly, the display apparatus of this embodiment can be used for display portions of electronic appliances such as a digital camera, a digital video camera, a digital photo frame, a mobile phone, a portable game console, a portable information terminal, and an audio reproducing device, in addition to display portions of electronic appliances with a relatively large screen, such as a television device, a desktop or laptop personal computer, a monitor of a computer or the like, digital signage, and a large game machine such as a pachinko machine.

### [Display Module]

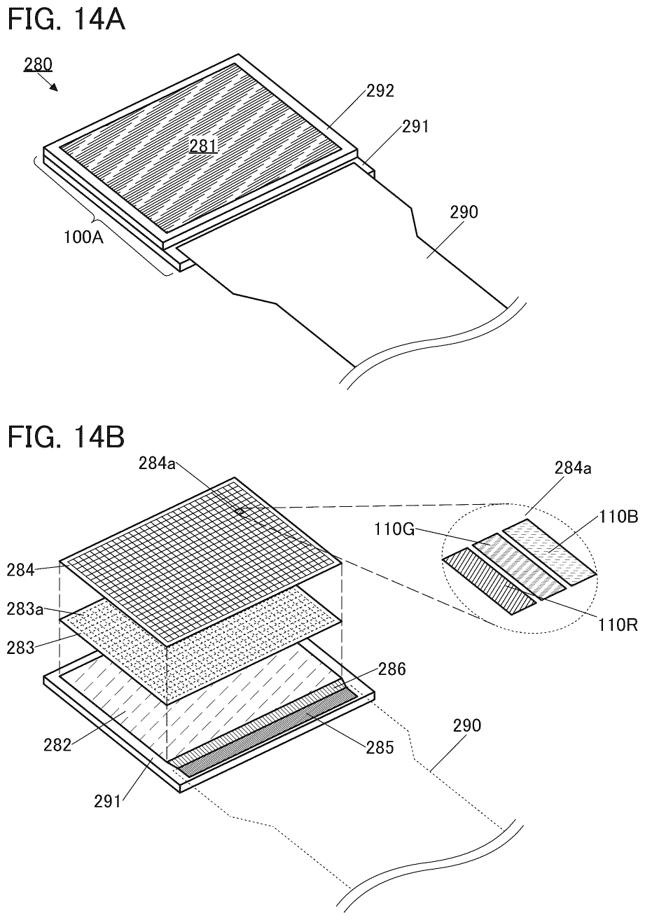

FIG. 14A is a perspective view of a display module 280. The display module 280 includes a display apparatus 100A and an FPC 290.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 14B is a perspective view schematically illustrating a structure on the substrate 291 side. Over the substrate 291, a circuit portion 282, a pixel circuit portion 283 over the circuit portion 282, and the pixel portion 284 over the pixel circuit portion 283 are stacked. A terminal portion 285 to be connected to the FPC 290 is provided over the substrate 291 in a portion that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side of FIG. 14B. The pixel 284a can employ any of the structures described in the above embodiments. FIG. 14B illustrates an example where the pixel 284a has a structure similar to that of the pixel 178 illustrated in FIG. 3.

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor for one light-emitting device. A gate signal is input to a gate of the selection transistor, and a video signal is input to a source or a drain of the selection transistor. Thus, an active-matrix display apparatus is achieved.

The circuit portion 282 includes a circuit for driving the pixel circuits 283a in the pixel circuit portion 283. For example, the circuit portion 282 preferably includes one or both of agate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure where one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high resolution. For example, the pixels 284a are preferably arranged in the display portion 281 with a resolution higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

Such a display module 280 has extremely high resolution, and thus can be suitably used for a VR device such as an HMD or a glasses-type AR device. For example, even in the case of a structure where the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be suitably used in a display portion of a wearable electronic appliance, such as a wrist watch.

### [Display Apparatus 100A]

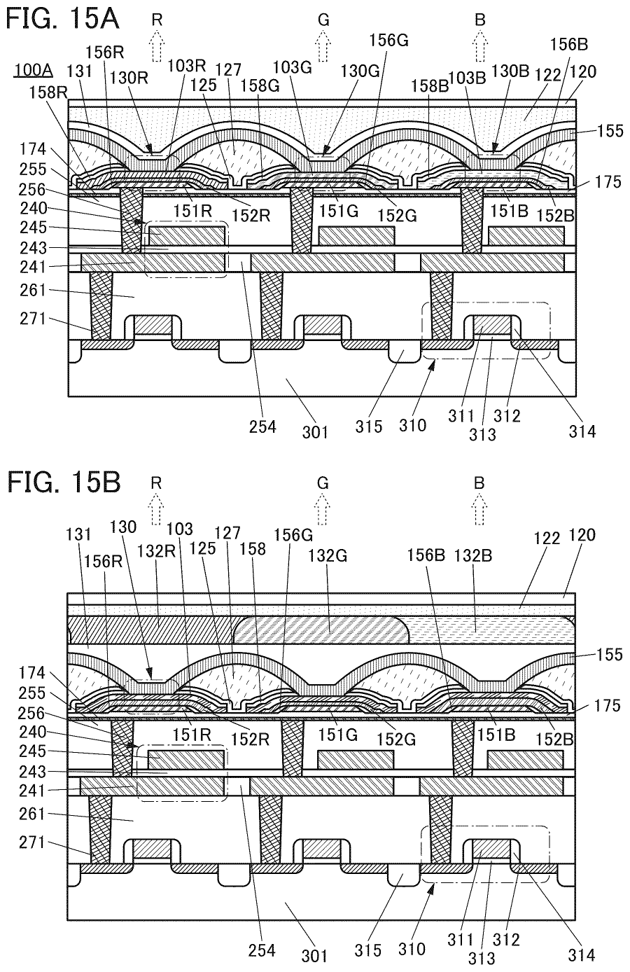

The display apparatus 100A illustrated in FIG. 15A includes a substrate 301, the light-emitting device 130R, the light-emitting device 130G, the light-emitting device 130B, a capacitor 240, and a transistor 310.

The substrate 301 corresponds to the substrate 291 in FIG. 14A and FIG. 14B. The transistor 310 is a transistor including a channel formation region in the substrate 301. As the substrate 301, a semiconductor substrate such as a single crystal silicon substrate can be used, for example. The transistor 310 includes part of the substrate 301, a conductive layer 311, low-resistance regions 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source and a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 positioned therebetween. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240, the insulating layer 174 is provided over the insulating layer 255, and the insulating layer 175 is provided over the insulating layer 174. The light-emitting device 130R, the light-emitting device 130G, and the light-emitting device 130B are provided over the insulating layer 175. FIG. 15A illustrates an example where the light-emitting device 130R, the light-emitting device 130G, and the light-emitting device 130B each have the stacked-layer structure illustrated in FIG. 6A. An insulator is provided in a region between adjacent light-emitting devices. In FIG. 15A, for example, the inorganic insulating layer 125 and the insulating layer 127 over the inorganic insulating layer 125 are provided in this region.

The insulating layer 156R is provided to include a region overlapping with the side surface of the conductive layer 151R of the light-emitting device 130R. The insulating layer 156G is provided to include a region overlapping with the side surface of the conductive layer 151G of the light-emitting device 130G. The insulating layer 156B is provided to include a region overlapping with the side surface of the conductive layer 151B of the light-emitting device 130B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R of the light-emitting device 130R. The sacrificial layer 158G is positioned over the organic compound layer 103G of the light-emitting device 130G. The sacrificial layer 158B is positioned over the organic compound layer 103B of the light-emitting device 130B.

Each of the conductive layer 151R, the conductive layer 151G, and the conductive layer 151B is electrically connected to one of the source and the drain of the corresponding transistor 310 through a plug 256 embedded in the insulating layer 243, the insulating layer 255, the insulating layer 174, and the insulating layer 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 131 is provided over the light-emitting device 130R, the light-emitting device 130G, and the light-emitting device 130B. The substrate 120 is attached onto the protective layer 131 with the resin layer 122. Embodiment 3 can be referred to for the details of the light-emitting devices 130 and the components thereover up to the substrate 120. The substrate 120 corresponds to the substrate 292 in FIG. 14A.

FIG. 15B illustrates a modification example of the display apparatus 100A illustrated in FIG. 15A. The display apparatus illustrated in FIG. 15B includes the coloring layer 132R, the coloring layer 132G, and the coloring layer 132B, and each of the light-emitting devices 130 includes a region overlapping with one of the coloring layer 132R, the coloring layer 132G, and the coloring layer 132B. In the display apparatus illustrated in FIG. 15B, the light-emitting device 130 can emit white light, for example. For example, the coloring layer 132R can transmit red light, the coloring layer 132G can transmit green light, and the coloring layer 132B can transmit blue light.

This embodiment can be combined as appropriate with any of the other embodiments or examples. In this specification, in the case where a plurality of structure examples are shown in one embodiment, the structure examples can be combined as appropriate.

### Embodiment 6

In this embodiment, electronic appliances of embodiments of the present invention will be described.

Electronic appliances of this embodiment each include the display apparatus of one embodiment of the present invention in a display portion. The display apparatus of one embodiment of the present invention is highly reliable and can be easily increased in resolution and definition. Thus, the display apparatus of one embodiment of the present invention can be used for a display portion of a variety of electronic appliances.

Examples of electronic appliances include a digital camera, a digital video camera, a digital photo frame, a mobile phone, a portable game machine, a portable information terminal, and an audio reproducing device in addition to electronic appliances with a relatively large screen, such as a television device, a desktop or laptop personal computer, a monitor of a computer or the like, digital signage, and a large game machine like a pachinko machine.

In particular, the display apparatus of one embodiment of the present invention can have high resolution, and thus can be suitably used for an electronic appliance including a relatively small display portion. Examples of such an electronic appliance include watch-type and bracelet-type information terminal devices (wearable devices) and wearable devices capable of being worn on a head, such as a VR device like a head-mounted display, a glasses-type AR device, and an MR device.

The definition of the display apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, the definition is preferably 4K, 8K, or higher. The pixel density (resolution) of the display apparatus of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. With the use of such a display apparatus having one or both of high definition and high resolution, the electronic appliance can provide higher realistic sensation, sense of depth, and the like in personal use such as portable use and home use. There is no particular limitation on the screen ratio (aspect ratio) of the display apparatus of one embodiment of the present invention. For example, the display apparatus is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance of this embodiment can have a variety of functions. For example, the electronic appliance of this embodiment can have a function of displaying a variety of data (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

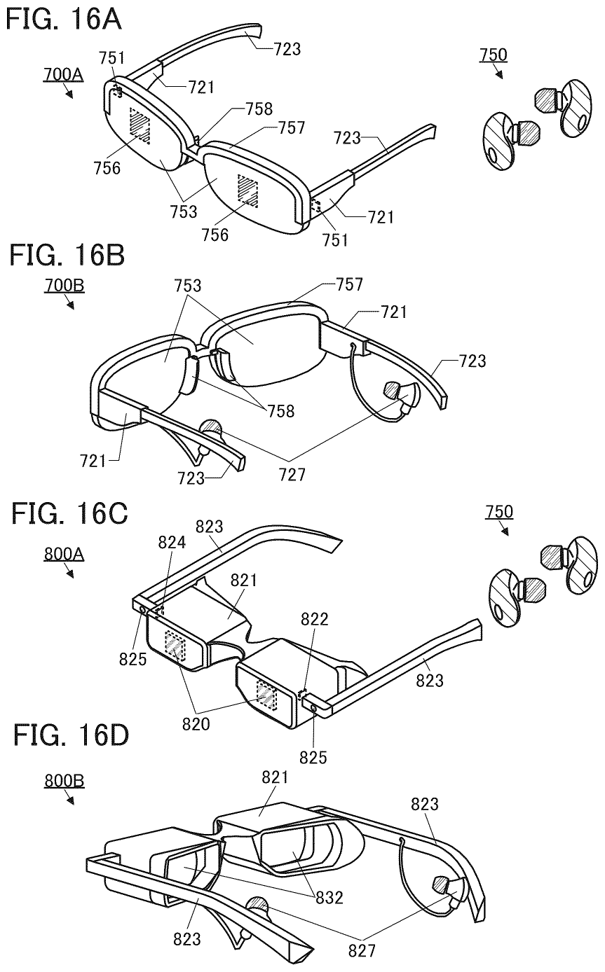

Examples of a wearable device capable of being worn on a head are described with reference to FIG. 16A to FIG. 16D. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic appliance 700A illustrated in FIG. 16A and an electronic appliance 700B illustrated in FIG. 16B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display apparatus of one embodiment of the present invention can be used for the display panel 751. Thus, a highly reliable electronic appliance can be obtained.

The electronic appliance 700A and the electronic appliance 700B can each project images displayed on the display panels 751 onto display regions 756 of the optical members 753. Since the optical members 753 have a light-transmitting property, a user can see images displayed on the display regions, which are superimposed on transmission images seen through the optical members 753. Accordingly, the electronic appliance 700A and the electronic appliance 700B are electronic appliances capable of AR display.

In the electronic appliance 700A and the electronic appliance 700B, a camera capable of capturing images of the front side may be provided as the image capturing portion. Furthermore, when the electronic appliance 700A and the electronic appliance 700B are provided with an acceleration sensor such as a gyroscope sensor, the orientation of the user's head can be sensed and an image corresponding to the orientation can be displayed on the display region 756.

The communication portion includes a wireless communication device, and a video signal, for example, can be supplied by the wireless communication device. Note that instead of the wireless communication device or in addition to the wireless communication device, a connector to which a cable for supplying a video signal and a power supply potential can be connected may be provided.

The electronic appliance 700A and the electronic appliance 700B are provided with a battery, so that they can be charged wirelessly and/or by wire.

A touch sensor module may be provided in the housing 721. The touch sensor module has a function of detecting touch on the outer surface of the housing 721. Detecting a tap operation, a slide operation, or the like by the user with the touch sensor module enables various types of processing. For example, processing such as a pause or a restart of a moving image can be executed by a tap operation, and processing such as fast forward or fast rewind can be executed by a slide operation. The touch sensor module is provided in each of the two housings 721, whereby the range of the operation can be increased.

Any of various touch sensors can be used for the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic appliance 800A illustrated in FIG. 16C and an electronic appliance 800B illustrated in FIG. 16D each include a pair of display portions 820, a housing 821, a communication portion 822, a pair of wearing portions 823, a control portion 824, a pair of image capturing portions 825, and a pair of lenses 832.

The display apparatus of one embodiment of the present invention can be used in the display portions 820. Thus, a highly reliable electronic appliance can be obtained.

The display portions 820 are provided at positions inside the housing 821 so as to be seen through the lenses 832. When the pair of display portions 820 display different images, three-dimensional display using parallax can be performed.

The electronic appliance 800A and the electronic appliance 800B can be regarded as electronic appliances for VR. The user wearing the electronic appliance 800A or the electronic appliance 800B can see images displayed on the display portions 820 through the lenses 832.

The electronic appliance 800A and the electronic appliance 800B each preferably include a mechanism for adjusting the lateral positions of the lenses 832 and the display portions 820 so that the lenses 832 and the display portions 820 are positioned optimally in accordance with the positions of the user's eyes. Moreover, the electronic appliance 800A and the electronic appliance 800B each preferably include a mechanism for adjusting focus by changing the distance between the lenses 832 and the display portions 820.

The electronic appliance 800A or the electronic appliance 800B can be mounted on the user's head with the wearing portions 823. Note that FIG. 16C illustrates an example where the wearing portion 823 has a shape like a temple (also referred to as a joint or the like) of glasses; however, one embodiment of the present invention is not limited thereto. The wearing portion 823 can have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided to cover a plurality of fields of view, such as a telescope field of view and a wide field of view.

Although an example where the image capturing portion 825 is provided is shown here, a range sensor (hereinafter also referred to as a sensing portion) capable of measuring a distance between the user and an object just needs to be provided. That is, the image capturing portion 825 is one embodiment of the sensing portion. As the sensing portion, an image sensor or a range image sensor such as a light detection and ranging (LiDAR) sensor can be used, for example. With the use of images obtained by the camera and images obtained by the distance image sensor, more pieces of information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliance 800A and the electronic appliance 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A illustrated in FIG. 16A has a function of transmitting information to the earphones 750 with the wireless communication function. As another example, the electronic appliance 800A illustrated in FIG. 16C has a function of transmitting information to the earphones 750 with the wireless communication function.

The electronic appliance may include an earphone portion. The electronic appliance 700B illustrated in FIG. 16B includes earphone portions 727. For example, the earphone portion 727 and the control portion can be connected to each other by wire. Part of a wiring that connects the earphone portion 727 and the control portion may be positioned inside the housing 721 or the wearing portion 723.

Similarly, the electronic appliance 800B illustrated in FIG. 16D includes earphone portions 827. For example, the earphone portion 827 and the control portion 824 can be connected to each other by wire. Part of a wiring that connects the earphone portion 827 and the control portion 824 may be positioned inside the housing 821 or the wearing portion 823. Alternatively, the earphone portions 827 and the wearing portions 823 may include magnets. This is preferable because the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of what is called a headset by including the audio input mechanism.

As described above, both the glasses-type device (e.g., the electronic appliance 700A and the electronic appliance 700B) and the goggles-type device (e.g., the electronic appliance 800A and the electronic appliance 800B) are preferable as the electronic appliance of one embodiment of the present invention.

The electronic appliance of one embodiment of the present invention can transmit information to earphones by wire or wirelessly.

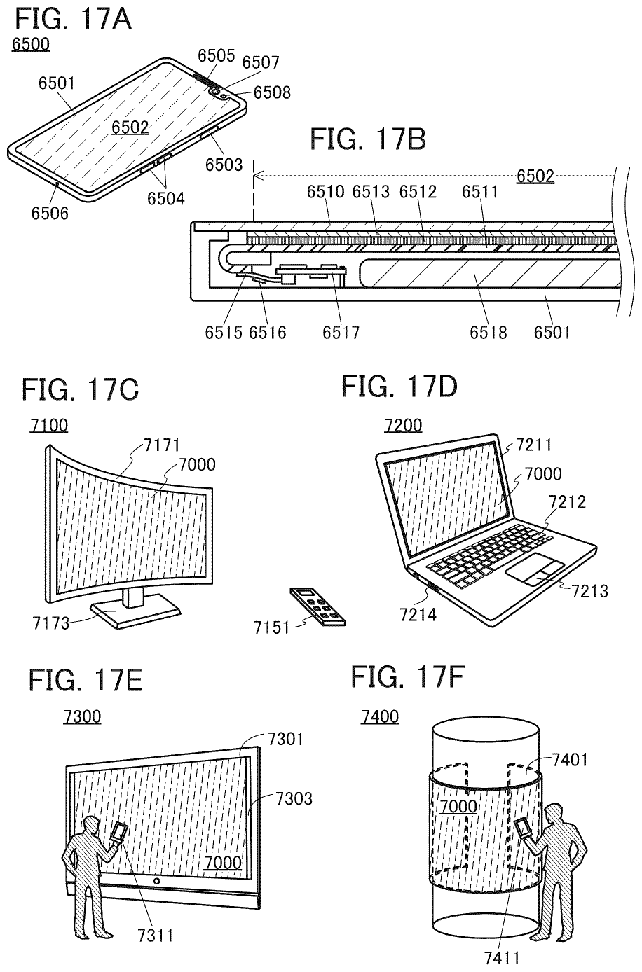

An electronic appliance 6500 illustrated in FIG. 17A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display apparatus of one embodiment of the present invention can be used in the display portion 6502. Thus, a highly reliable electronic appliance can be obtained.

FIG. 17B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

A flexible display of one embodiment of the present invention can be used as the display panel 6511. Thus, an extremely lightweight electronic appliance can be achieved. Since the display panel 6511 is extremely thin, the battery 6518 with high capacity can be mounted while an increase in thickness of the electronic appliance is suppressed. Moreover, part of the display panel 6511 is folded back so that a connection portion with the FPC 6515 is provided on the back side of a pixel portion, whereby an electronic appliance with a narrow bezel can be achieved.

FIG. 17C illustrates an example of a television device. In a television device 7100, a display portion 7000 is incorporated in a housing 7171. Here, the housing 7171 is supported by a stand 7173.

The display apparatus of one embodiment of the present invention can be used in the display portion 7000. Thus, a highly reliable electronic appliance can be obtained.

Operation of the television device 7100 illustrated in FIG. 17C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel provided in the remote control 7151, channels and volume can be controlled and videos displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 17D illustrates an example of a laptop personal computer. A laptop personal computer 7200 includes a housing 7211, a keyboard 7212, a pointing device 7213, an external connection port 7214, and the like. In the housing 7211, the display portion 7000 is incorporated.

The display apparatus of one embodiment of the present invention can be used in the display portion 7000. Thus, a highly reliable electronic appliance can be obtained.

FIG. 17E and FIG. 17F illustrate examples of digital signage.

Digital signage 7300 illustrated in FIG. 17E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 17F is digital signage 7400 attached to a cylindrical pillar 7401. The digital signage 7400 includes the display portion 7000 provided along a curved surface of the pillar 7401.

The display apparatus of one embodiment of the present invention can be used in the display portion 7000 illustrated in each of FIG. 17E and FIG. 17F. Thus, a highly reliable electronic appliance can be obtained.

A larger area of the display portion 7000 can increase the amount of information that can be provided at a time. The larger display portion 7000 attracts more attention, so that the effectiveness of the advertisement can be increased, for example.

A touch panel is preferably used in the display portion 7000, in which case intuitive operation by a user is possible in addition to display of an image or a moving image on the display portion 7000. Moreover, for an application for providing information such as route information or traffic information, usability can be enhanced by intuitive operation.

As illustrated in FIG. 17E and FIG. 17F, it is preferable that the digital signage 7300 or the digital signage 7400 can work with an information terminal 7311 or an information terminal 7411 such as a smartphone a user has through wireless communication. For example, information of an advertisement displayed on the display portion 7000 can be displayed on a screen of the information terminal 7311 or the information terminal 7411. By operation of the information terminal 7311 or the information terminal 7411, display on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

This embodiment can be combined as appropriate with any of the other embodiments or examples. In this specification, in the case where a plurality of structure examples are shown in one embodiment, the structure examples can be combined as appropriate.

### Example 1

### Synthesis Example 1

In this example, a synthesis method of 1-(3′,4′,5′-triphenyl-1,1′:2′,1″-terphenyl-3-yl)-1,3,4,6,7,8-hexahydro-2H-pyrimide[1,2-a]pyrimidine (abbreviation: Ph3TP-hpp) (Structural Formula (100)), which is the organic compound of one embodiment of the present invention, is described.

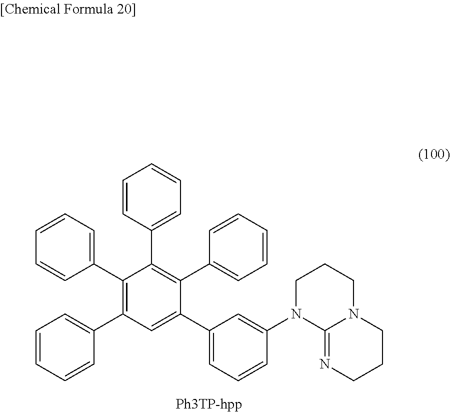 [SMILES: c1ccc(-c2cc(-c3cccc(N4CCCN5CCCN=C54)c3)c(-c3ccccc3)c(-c3ccccc3)c2-c2ccccc2)cc1]

### <Synthesis of Ph3TP-hpp>

Into a 300-mL three-neck flask were put 4.5 g (8.4 mmol) of 3-bromo-3′,4′,5′-triphenyl-1,1′:2′,1″-terphenyl, 1.5 g (11 mmol) of 1,3,4,6,7,8-hexahydro-2H-pyrimide[1,2-a]pyrimidine, 0.31 mg (0.50 mmol) of (±)-2,2′-bis(diphenylphosphino)-1,1′-binaphthyl (abbreviation: rac-BINAP), 2.4 g (21 mmol) of potassium-tert-butoxide, and 84 mL of dehydrated toluene, and the mixture was degassed by being stirred under reduced pressure. To this mixture was added 75 mg (0.33 mmol) of palladium(II) acetate, and the mixture was stirred at 90° C. under a nitrogen stream for 12 hours. After the stirring, the mixture was cooled down to room temperature. An insoluble matter was removed from the mixture by suction filtration, and the obtained filtrate was subjected to extraction with toluene. The extracted solution was concentrated to give a solid. A small amount of toluene was added to the solid, irradiation with ultrasonic waves was performed, and a solid was collected by suction filtration, whereby a target yellow solid was obtained (0.27 g, in a yield of 5.4%). The synthesis scheme is shown in Formula (a-1) below.

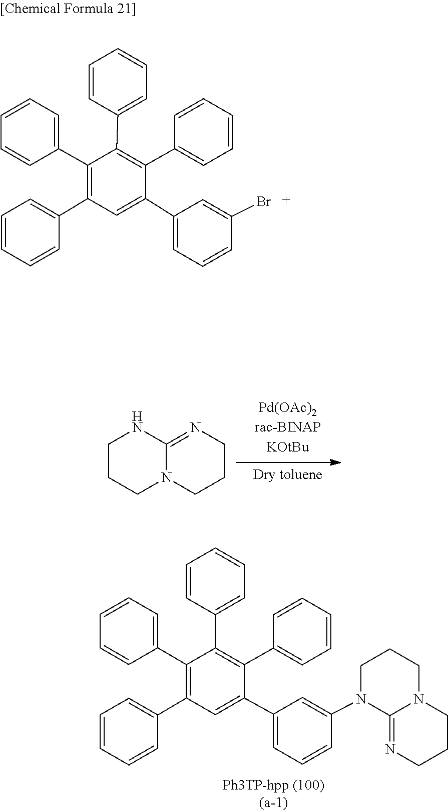 [SMILES: Brc1cccc(-c2cc(-c3ccccc3)c(-c3ccccc3)c(-c3ccccc3)c2-c2ccccc2)c1 ; C1CN=C2NCCCN2C1 ; c1ccc(-c2cc(-c3cccc(N4CCCN5CCCN=C54)c3)c(-c3ccccc3)c(-c3ccccc3)c2-c2ccccc2)cc1]

By a train sublimation method, 0.27 g of the obtained yellow solid was purified by sublimation. In the purification by sublimation, heating was performed for 20 hours at an argon flow rate of 5 mL/min, a pressure of 2.8 Pa, and a heating temperature of 210° C. As a result, a target white solid was obtained (0.14 g, at a collection rate of 52%).

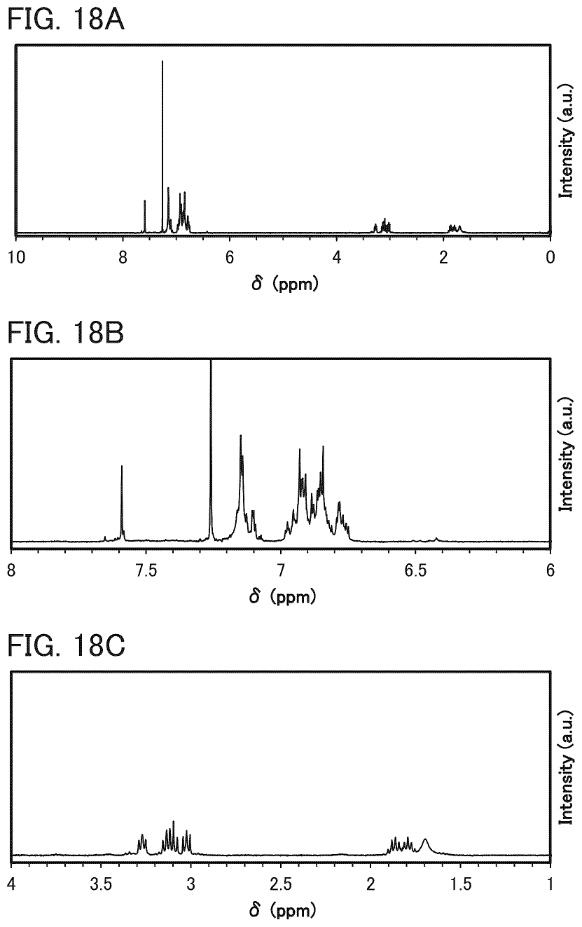

FIG. 18A to FIG. 18C show the ¹H NMR spectra of Ph3TP-hpp after the purification by sublimation. Results of ¹H NMR measurement are shown below. The results show that Ph3 TP-hpp was obtained.

¹H NMR (CDCl₃, 300 MHz): δ=7.59 (s, 1H), 7.16-6.75 (m, 24H), 3.27 (t, J=5.7 Hz, 2H), 3.16-3.08 (m, 4H), 3.02 (t, J=5.9 Hz, 2H), 1.90-1.75 (m, 4H).

The glass transition temperature (Tg) of Ph3TP-hpp was measured. The T_{g} was measured with a differential scanning calorimeter (DSC8500 produced by PerkinElmer Japan Co., Ltd.) in a state where a powder was put on an aluminum cell and the temperature was increased at a rate of 40° C./min. As a result, the T_{g} of Ph3TP-hpp was 107° C.

### <Solubility Test of Ph3TP-Hpp by LC/MS Analysis>

In the LC/MS analysis, LC (liquid chromatography) separation was carried out with Acquity UPLC (registered trademark) produced by Waters Corporation, and MS analysis (mass spectrometry) was carried out with Xevo G2 Tof MS produced by Waters Corporation. Acquity UPLC BEH C8 (2.1×100 mm, 1.7 μm) was used as a column for the LC separation. Acetonitrile was used for Mobile Phase A and a 0.1% aqueous solution of formic acid was used for Mobile Phase B. The injection amount of the sample was 5.0 μL. Note that in the analysis, the wavelength of a photodiode array detector was set to 246 nm±1 nm.

In a 5-mL sample bottle, 1 mg of Ph3TP-hpp was put and 2 mL of toluene was added thereto, and then this mixture was irradiated with ultrasonic waves for five minutes. After it was confirmed that the solid was completely dissolved, this solution was diluted by 2.5 times with acetonitrile, whereby the concentration of the solution was adjusted to 200 mg/L. This solution was diluted with acetonitrile, whereby solutions with concentrations of 20 mg/L, 10 mg/L, and 4 mg/L were prepared. The prepared solutions were subjected to LC/MS analysis, and the peak area values derived from Ph3TP-hpp, which were obtained at the respective solution concentrations, were used to form calibration curves.

Next, the water solubility of Ph3TP-hpp was measured.

In a 5-mL sample bottle, 1 mg of Ph3TP-hpp was put and 1 mL of water was added thereto, and then this mixture was irradiated with ultrasonic waves for five minutes. This mixture was filtered through a membrane filter to remove the solid, and the obtained filtrate was diluted by five times with acetonitrile. The obtained solution was subjected to LC/MS analysis.

From the calibration curve and the signal intensity obtained by the LC/MS analysis, it is found that the solubility of Ph3TP-hpp dissolved in 1 mL of water is 0.031 mg. The weight fraction of the water solubility of Ph3TP-hpp is therefore 3.1×10⁻⁵ g/mL.

The above results reveal that Ph3TP-hpp is an organic compound having low water solubility.

It is thus found that the organic compound of one embodiment of the present invention can be favorably used for a light-emitting device whose fabrication process includes processing using water or a chemical solution containing water as a solvent (i.e., a light-emitting device involving processing by a lithography method).

### Example 2

In this example, a light-emitting device 1 fabricated using Ph3TP-hpp (Structural Formula (100)), which is the organic compound of one embodiment of the present invention, is described. Note that the light-emitting device 1 is a light-emitting device where an organic compound layer is processed by a photolithography method. Structural formulae of organic compounds used for the light-emitting device 1 are shown below.

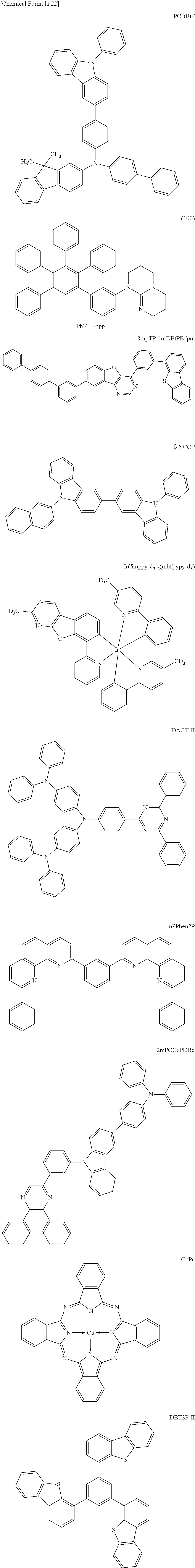

### (Method for Fabricating Light-Emitting Device 1)

First, as a reflective electrode, an alloy containing silver (Ag), palladium (Pd), and copper (Cu) (abbreviation: APC) was formed over a glass substrate to a thickness of 100 nm by a sputtering method, and then, as a transparent electrode, an indium tin oxide containing silicon oxide (ITSO) was formed to a thickness of 100 nm by a sputtering method, whereby the first electrode 101 was formed. The electrode area was set to 4 mm² (2 mm×2 mm). Note that the transparent electrode functions as an anode, and the transparent electrode and the reflective electrode can be collectively regarded as the first electrode.

Next, in pretreatment for forming the light-emitting device over the substrate, the surface of the substrate was washed with water and baking was performed at 200° C. for 1 hour, and then UV ozone treatment was performed for 370 seconds.

After that, the substrate was transferred into a vacuum evaporation apparatus where the pressure was reduced to approximately 1×10⁻⁴ Pa, heat treatment was performed at 170° C. for 30 minutes in a heating chamber of the vacuum evaporation apparatus, and then the substrate was cooled down for approximately 30 minutes.

Then, the substrate provided with the first electrode was fixed to a holder provided in the vacuum evaporation apparatus such that the surface over which the first electrode was formed faced downward. Over the first electrode, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron-acceptor material with a molecular weight of 672 (OCHD-003) were deposited by co-evaporation to a thickness of 10 nm at a weight ratio of 1:0.03 (=PCBBiF:OCHD-003), whereby a hole-injection layer was formed.

Over the hole-injection layer, PCBBiF was deposited by evaporation to a thickness of 60 nm, whereby a first hole-transport layer was formed.

Then, over the first hole-transport layer, 8-(1,1′:4,1″-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-9H,9′H-3,3′-bicarbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN2)phenyl-κC]iridium(III) (abbreviation: Ir(5mppy-d₃)₂(mbfpypy-d₃)) were deposited by co-evaporation to a thickness of 40 nm at a weight ratio of 0.5:0.5:0.1 (=8mpTP-4mDBtPBfpm: QNCCP: Ir(5mppy-d₃)₂(mbfpypy-d₃)), whereby a first light-emitting layer was formed.

Next, 3,6-bis(diphenylamino)-9-[4-(4,6-diphenyl-1,3,5-triazin-2-yl)phenyl]-9H-carbazole (abbreviation: DACT-II) was deposited by evaporation to a thickness of 10 nm, whereby a first electron-transport layer was formed.

After formation of the first electron-transport layer, 2,2′-(1,3-phenylene)bis(9-phenyl-1,10-phenanthroline) (abbreviation: mPPhen2P) and Ph3TP-hpp (Structural Formula (100)), which is the organic compound of one embodiment of the present invention, were deposited by co-evaporation to a thickness of 5 nm at a weight ratio of 5:5 (=mPPhen2P: Ph3TP-hpp), whereby a first layer was formed. Then, copper phthalocyanine (abbreviation: CuPc) was deposited to a thickness of 2 nm, whereby a third layer was formed. Furthermore, PCBBiF and OCHD-003 were deposited by co-evaporation to a thickness of 10 nm at a weight ratio of 1:0.15 (=PCBBiF: OCHD-003) to form a second layer, whereby an intermediate layer including the first layer, the second layer, and the third layer between the first layer and the second layer was formed.

Next, over the intermediate layer, PCBBiF was deposited by evaporation to a thickness of 55 nm, whereby a second hole-transport layer was formed.

Over the second hole-transport layer, 8mpTP-4mDBtPBfpm, βNCCP, and Ir(5mppy-d₃)₂(mbfpypy-d₃) were deposited by co-evaporation to a thickness of 40 nm at a weight ratio of 0.5:0.5:0.1 (=8mpTP-4mDBtPBfpm: βNCCP: Ir(5mppy-d₃)₂(mbfpypy-d₃)), whereby a second light-emitting layer was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 20 nm, and mPPhen2P was further deposited by evaporation to a thickness of 20 nm, whereby a second electron-transport layer was formed.

After formation of the second electron-transport layer, processing by a photolithography method and heat treatment were performed.

### <<Processing by Photolithography Method and Heat Treatment>>

Here, the processing by a photolithography method and the heat treatment are described. First, the substrate was taken out from the vacuum evaporation apparatus and exposed to the air, and then aluminum oxide was deposited to a thickness of 30 nm by an ALD method using trimethylaluminum (abbreviation: TMA) as a precursor and water vapor as an oxidizer, whereby a first sacrificial layer was formed.

Then, over the first sacrificial layer, a composite oxide containing indium, gallium, zinc, and oxygen (abbreviation: IGZO) was deposited to a thickness of 50 nm by a sputtering method, whereby a second sacrificial layer was formed.

A resist was formed using a photoresist over the second sacrificial layer, and processing was performed by a lithography method to form a slit having a width of 3 μm in a position 3.5 μm away from an end portion of the first electrode.

Specifically, the second sacrificial layer was processed using a chemical solution containing a phosphoric acid aqueous solution with the use of the resist as a mask, and then the first sacrificial layer was processed using an etching gas containing fluoroform (CHF₃) and helium (He) at a flow rate ratio of CHF₃:He=1:9. Then, the second electron-transport layer, the second light-emitting layer, the second hole-transport layer, the intermediate layer, the first electron-transport layer, the first light-emitting layer, the first hole-transport layer, and the hole-injection layer were processed using an etching gas containing oxygen (O₂).

After the processing by a photolithography method, the second sacrificial layer and the first sacrificial layer were removed using a basic chemical solution containing water as a solvent, so that the top surface of the second electron-transport layer was exposed. Then, the substrate was transferred into a vacuum evaporation apparatus where the pressure was reduced to approximately 1×10⁻⁴ Pa, and heat treatment was performed at 110° C. for 1 hour in a heating chamber of the vacuum evaporation apparatus.

The above is the description of the processing by a photolithography method and the heat treatment. As described above, in the processing by a photolithography method and the heat treatment, treatment using water or a chemical solution containing water as a solvent is performed.

After the processing by a photolithography method and the heat treatment, over the exposed second electron-transport layer, lithium fluoride (LiF) and ytterbium (Yb) were deposited by co-evaporation to a thickness of 1.5 nm at a volume ratio of 2:1 (=LiF:Yb) to form an electron-injection layer, and lastly silver (Ag) and magnesium (Mg) were deposited by co-evaporation to a thickness of 15 nm at a volume ratio of 1:0.1 to form a second electrode, whereby the light-emitting device 1 was fabricated.

The second electrode is a transflective electrode, which has a function of reflecting light and a function of transmitting light, and the light-emitting device of this example is atop-emission tandem light-emitting device from which light is extracted through the second electrode. Over the second electrode, 4,4′,4″-(benzene-1,3,5-triyl)tri(dibenzothiophene) (abbreviation: DBT3P-II) was deposited by evaporation to a thickness of 70 nm as a cap layer so that light extraction efficiency can be improved.

The device structure of the light-emitting device 1 is shown in the following table.

TABLE 1

| | | Thickness | | Light-emitting device 1 |
|---|---|---|---|---|
| Cap layer | | 70 | nm | DBT3P-II |
| Second electrode | | 15 | nm | Ag:Mg (1:0.1) |
| Electron-injection layer | | 1.5 | nm | LiF:Yb (2:1) |
| Processing by photolithography method | | | | Processing by photolithography method is performed. |
| Second electron- | 2 | 20 | nm | mPPhen2P |
| transport layer | 1 | 20 | nm | 2mPCCzPDBq |
| Second light-emitting layer | | 40 | nm | 8mpTP-4mDBtPBfpm:βNCCP:Ir(5mppy-d₃)₂(mbfpypy-d₃) |
| | | | | (0.5:0.5:0.1) |
| Second hole-transport layer | | 55 | nm | PCBBiF |
| Intermediate | Second layer | 10 | nm | PCBBiF:OCHD-003 (1:0.15) |
| layer | Third layer | 2 | nm | CuPc |
| | First layer | 5 | nm | mPPhen2P:Ph3TP-hpp (5:5) |
| First electron-transport layer | | 10 | nm | DACT-II |
| First light-emitting layer | | 40 | nm | 8mpTP-4mDBtPBfpm:βNCCP:Ir(5mppy-d3)2(mbfpypy-d3) |
| | | | | (0.5:0.5:0.1) |
| First hole-transport layer | | 60 | nm | PCBBiF |
| Hole-injection layer | | 10 | nm | PCBBiF:OCHD-003 (1:0.03) |
| First electrode | 2 | 100 | nm | ITSO |
| | 1 | 100 | nm | APC |

The light-emitting device 1 was sealed using a glass substrate in a glove box containing a nitrogen atmosphere so as not to be exposed to the air (a UV curable sealing material was applied to surround the element, only the sealing material was irradiated with UV while the light-emitting device was prevented from being irradiated with the UV, and heat treatment was performed at 80° C. under an atmospheric pressure for one hour). Then, the initial characteristics of the light-emitting device were measured.

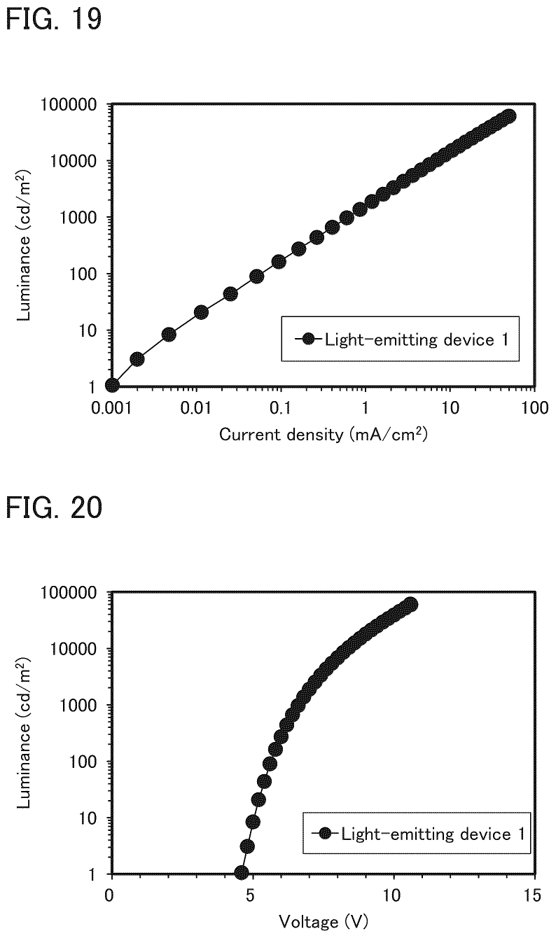

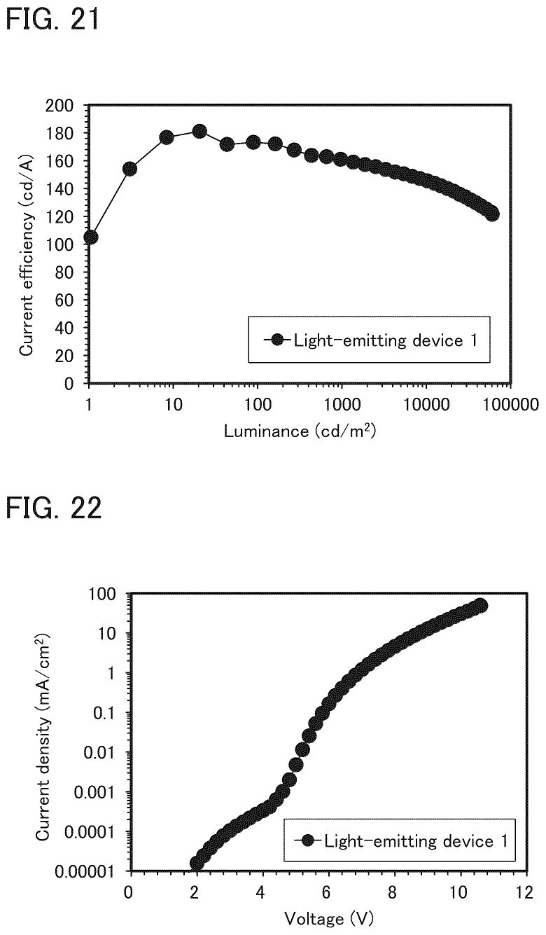

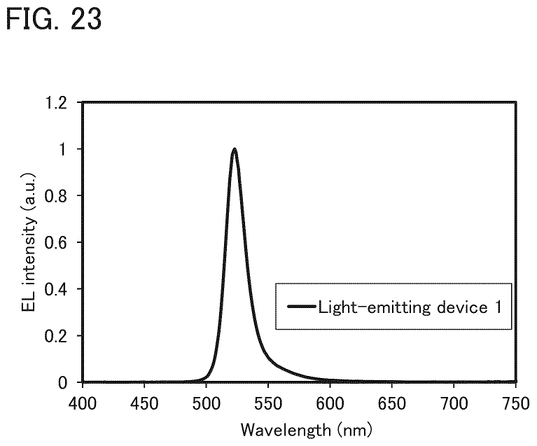

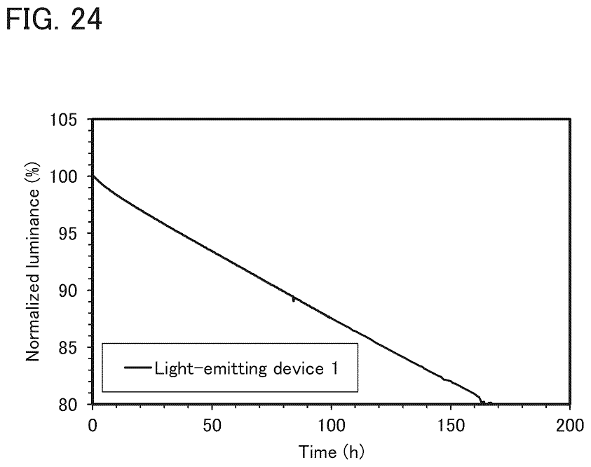

FIG. 19 shows the luminance-current density characteristics of the light-emitting device 1. FIG. 20 shows the luminance-voltage characteristics thereof. FIG. 21 shows the current efficiency-luminance characteristics thereof. FIG. 22 shows the current density-voltage characteristics thereof. FIG. 23 shows the electroluminescence spectrum thereof. FIG. 24 shows a luminance change over driving time when the light-emitting device 1 was driven at a constant current of 2 mA (50 mA/cm²).

The table below shows the main characteristics of the light-emitting device 1 at a luminance of around 1000 cd/m². Note that the luminance, CIE chromaticity, and electroluminescence spectrum were measured at normal temperature with a spectroradiometer (SR-UL1R produced by TOPCON CORPORATION).

TABLE 2

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Light-emitting | 6.6 | 0.024 | 0.601 | 0.173 | 0.763 | 968 | 161 |
| device 1 | | | | | | | |

FIG. 19 to FIG. 24 and the above table reveal that the light-emitting device 1 exhibits green light emission derived from Ir(5mppy-d₃)₂(mbfpypy-d₃) and has favorable light-emitting characteristics. In addition, the light-emitting device 1 has high current efficiency and is driven as a tandem light-emitting device. This indicates that the use of the organic compound of one embodiment of the present invention for an intermediate layer enables a light-emitting device having favorable characteristics to be fabricated.

Since the organic compound of one embodiment of the present invention is an organic compound having low water solubility, problems such as dissolution of a layer including the organic compound and permeation of a chemical solution into the layer including the organic compound can be prevented even when treatment using water is included in a manufacturing process of the light-emitting device including the organic compound of one embodiment of the present invention. Moreover, the organic compound of one embodiment of the present invention has a T_{g} higher than or equal to 100° C., thereby preventing abnormality in film quality in a heating step. Furthermore, as shown in this example, it is found that the use of the organic compound of one embodiment of the present invention for an intermediate layer of a light-emitting device enables the light-emitting device to have favorable characteristics. Thus, it can be said that, in manufacture of a light-emitting device whose manufacturing process includes treatment using water, the use of the organic compound of one embodiment of the present invention for an intermediate layer enables the light-emitting device to have favorable characteristics while preventing degraded characteristics, a shape defect, or the like of the light-emitting device, as compared with the case of using an organic compound having high water solubility for the intermediate layer and the case of using an organic compound having a low T_{g} for the intermediate layer.

### REFERENCE NUMERALS

100A: display apparatus, 100: display apparatus, 101: first electrode, 101a: first electrode, 101b: first electrode, 102: second electrode, 103: organic compound layer, 103a: organic compound layer, 103b: organic compound layer, 103B: organic compound layer, 103Bf: organic compound film, 103G: organic compound layer, 103Gf: organic compound film, 103R: organic compound layer, 103Rf: organic compound film, 104: common layer, 110B: subpixel, 110G: subpixel, 110R: subpixel, 110: subpixel, 111: hole-injection layer, 111a: hole-injection layer, 111b: hole-injection layer, 112: hole-transport layer, 112_1: first hole-transport layer, 112a_1: first hole-transport layer, 112b_1: first hole-transport layer, 112_2: second hole-transport layer, 112a_2: second hole-transport layer, 112b_2: second hole-transport layer, 113_1: first light-emitting layer, 113a_1: first light-emitting layer, 113b_1: first light-emitting layer, 113_2: second light-emitting layer, 113a_2: second light-emitting layer, 113b_2: second light-emitting layer, 113: light-emitting layer, 114: electron-transport layer, 114_1: first electron-transport layer, 114a_1: first electron-transport layer, 114b_1: first electron-transport layer, 114_2: second electron-transport layer, 114a_2: second electron-transport layer, 114b_2: second electron-transport layer, 115: electron-injection layer, 116_1: first intermediate layer, 116_2: second intermediate layer, 116: intermediate layer, 116a: intermediate layer, 116b: intermediate layer, 117: second layer, 117a: second layer, 117b: second layer, 118: third layer, 118a: third layer, 118b: third layer, 119: first layer, 119a: first layer, 119b: first layer, 120: substrate, 122: resin layer, 125f: inorganic insulating film, 125: inorganic insulating layer, 127a: insulating layer, 127f: insulating film, 127: insulating layer, 130B: light-emitting device, 130G: light-emitting device, 130R: light-emitting device, 130: light-emitting device, 130a: light-emitting device, 130b: light-emitting device, 131: protective layer, 132B: coloring layer, 132G: coloring layer, 132R: coloring layer, 140: connection portion, 141: region, 151a: conductive layer, 151B: conductive layer, 151b: conductive layer, 151C: conductive layer, 151c: conductive layer, 151f: conductive film, 151G: conductive layer, 151R: conductive layer, 151: conductive layer, 152a: conductive layer, 152B: conductive layer, 152b: conductive layer, 152C: conductive layer, 152c: conductive layer, 152f: conductive film, 152G: conductive layer, 152R: conductive layer, 152: conductive layer, 155: common electrode, 156B: insulating layer, 156C: insulating layer, 156f: insulating film, 156G: insulating layer, 156R: insulating layer, 156: insulating layer, 158: sacrificial layer, 158B: sacrificial layer, 158Bf: sacrificial film, 158G: sacrificial layer, 158Gf: sacrificial film, 158R: sacrificial layer, 158Rf: sacrificial film, 159B: mask layer, 159Bf: mask film, 159G: mask layer, 159Gf: mask film, 159R: mask layer, 159Rf: mask film, 171: insulating layer, 172: conductive layer, 173: insulating layer, 174: insulating layer, 175: insulating layer, 176: plug, 177: pixel portion, 178: pixel, 179: conductive layer, 190B: resist mask, 190G: resist mask, 190R: resist mask, 191: resist mask, 240: capacitor, 241: conductive layer, 243: insulating layer, 245: conductive layer, 254: insulating layer, 255: insulating layer, 256: plug, 261: insulating layer, 271: plug, 280: display module, 281: display portion, 282: circuit portion, 283a: pixel circuit, 283: pixel circuit portion, 284a: pixel, 284: pixel portion, 285: terminal portion, 286: wiring portion, 290: FPC, 291: substrate, 292: substrate, 301: substrate, 310: transistor, 311: conductive layer, 312: low-resistance region, 313: insulating layer, 314: insulating layer, 315: element isolation layer, 450 base film, 451a organic compound layer, 451s surface, 451 organic compound film, 452a organic mask layer, 452 organic mask film, 453a inorganic mask layer, 453r inorganic mask residue, 453 inorganic mask film, 454a hard mask layer, 454 film, 455a photomask layer, 455 resin film, 501: first light-emitting unit, 501a: first light-emitting unit, 501b: first light-emitting unit, 502: second light-emitting unit, 502a: second light-emitting unit, 502b: second light-emitting unit, 503: third light-emitting unit, 700A: electronic appliance, 700B: electronic appliance, 721: housing, 723: wearing portion, 727: earphone portion, 750: earphone, 751: display panel, 753: optical member, 756: display region, 757: frame, 758: nose pad, 800A: electronic appliance, 800B: electronic appliance, 820: display portion, 821: housing, 822: communication portion, 823: wearing portion, 824: control portion, 825: image capturing portion, 827: earphone portion, 832: lens, 6500: electronic appliance, 6501: housing, 6502: display portion, 6503: power source button, 6504: button, 6505: speaker, 6506: microphone, 6507: camera, 6508: light source, 6510: protective member, 6511: display panel, 6512: optical member, 6513: touch sensor panel, 6515: FPC, 6516: IC, 6517: printed circuit board, 6518: battery, 7000: display portion, 7100: television device, 7151: remote control, 7171: housing, 7173: stand, 7200: laptop personal computer, 7211: housing, 7212: keyboard, 7213: pointing device, 7214: external connection port, 7300: digital signage, 7301: housing, 7303: speaker, 7311: information terminal, 7400: digital signage, 7401: pillar, 7411: information terminal

1. An organic compound represented by General Formula (G1), 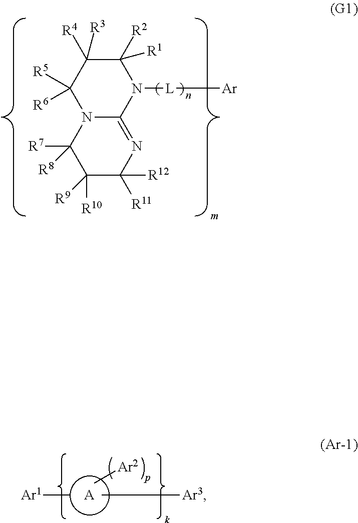
wherein):
Ar is an aromatic skeleton represented by General Formula (Ar-1);
L represents any one of an alkylene group having 1 to 10 carbon atoms, a substituted or unsubstituted arylene group having 6 to 30 carbon atoms, and a substituted or unsubstituted heteroarylene group having 1 to 11 carbon atoms;
n represents an integer greater than or equal to 0 and less than or equal to 3;
m represents an integer greater than or equal to 1 and less than or equal to 6;
each of R¹ to R¹² independently represents hydrogen or an alkyl group having 1 to 10 carbon atoms;
a ring A represents a benzene ring or a naphthalene ring;
each of Ar¹ to Ar³ independently represents a substituted or unsubstituted aryl group having 6 to 30 carbon atoms or a substituted or unsubstituted heteroaryl group having 1 to 30 carbon atoms, and any m carbon atoms contained in the ring A and Ar¹ to Ar³ are bonded to m bonds in General Formula (G1);
k represents an integer greater than or equal to 1 and less than or equal to 3; and
p represents an integer greater than or equal to 2 and less than or equal to 6.

2. An organic compound represented by General Formula (G1), 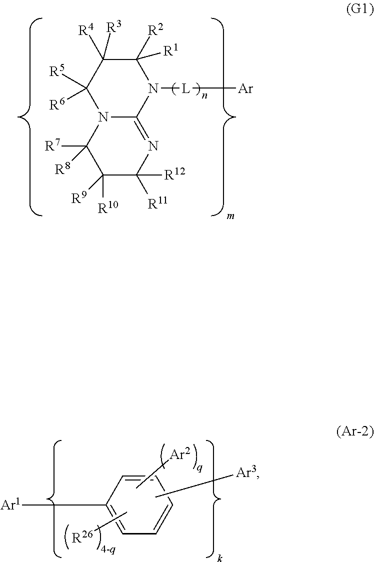
wherein:
Ar is an aromatic skeleton represented by General Formula (Ar-2);
L represents any one of an alkylene group having 1 to 10 carbon atoms, a substituted or unsubstituted arylene group having 6 to 30 carbon atoms, and a substituted or unsubstituted heteroarylene group having 1 to 11 carbon atoms;
n represents an integer greater than or equal to 0 and less than or equal to 3;
m represents an integer greater than or equal to 1 and less than or equal to 6;
each of R¹ to R¹² independently represents hydrogen or an alkyl group having 1 to 10 carbon atoms;
each of Ar¹ to Ar³ independently represents a substituted or unsubstituted aryl group having 6 to 30 carbon atoms or a substituted or unsubstituted heteroaryl group having 1 to 30 carbon atoms, and any m carbon atoms contained in Ar¹ to Ar³ are bonded to m bonds in General Formula (G1);
R²⁶ represents any one of hydrogen, an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms;
k represents an integer greater than or equal to 1 and less than or equal to 3; and
q represents an integer greater than or equal to 2 and less than or equal to 4.

3. An organic compound represented by General Formula (G1), 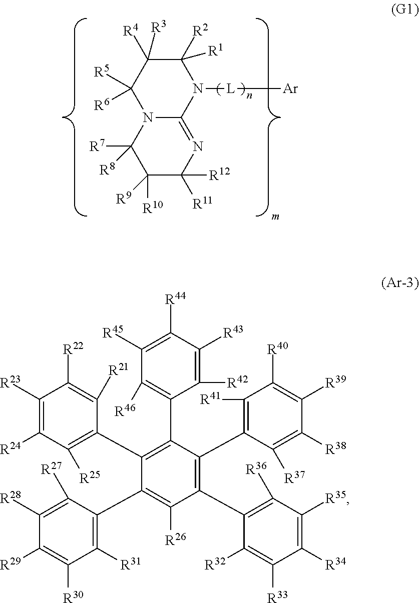
wherein:
Ar is an aromatic skeleton represented by General Formula (Ar-3);
L represents any one of an alkylene group having 1 to 10 carbon atoms, a substituted or unsubstituted arylene group having 6 to 30 carbon atoms, and a substituted or unsubstituted heteroarylene group having 1 to 11 carbon atoms;
n represents an integer greater than or equal to 0 and less than or equal to 3;
m represents an integer greater than or equal to 1 and less than or equal to 6; and
each of R¹ to R¹² independently represents hydrogen or an alkyl group having 1 to 10 carbon atoms,
any m groups of R²¹ to R⁴⁶ represent m bonding positions in General Formula (G1); and
each of the other groups of R²¹ to R⁴⁶ independently represents any one of hydrogen, an alkyl group having 1 to 10 carbon atoms, a cycloalkyl group having 3 to 10 carbon atoms, a substituted or unsubstituted aryl group having 6 to 30 carbon atoms, and a substituted or unsubstituted heteroaryl group having 1 to 30 carbon atoms.

4. The organic compound according to claim 3, wherein the organic compound is represented by General Formula (G2),

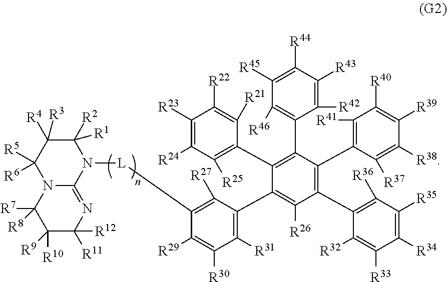

5. The organic compound according to claim 3, wherein the organic compound is represented by Structural Formula (100),

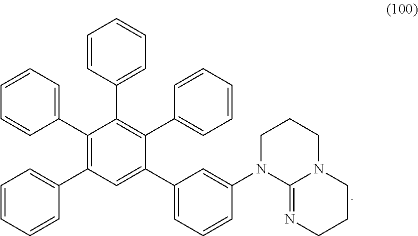 [SMILES: c1ccc(-c2cc(-c3cccc(N4CCCN5CCCN=C54)c3)c(-c3ccccc3)c(-c3ccccc3)c2-c2ccccc2)cc1]

6. A light-emitting device comprising the organic compound according to claim 1.

7. A display apparatus comprising:
the light-emitting device according to claim 6, and at least one of a transistor and a substrate.

8. An electronic appliance comprising:
the display apparatus according to claim 7, and at least one of a sensor portion, an input portion, and a communication portion.

9. A light-emitting device comprising the organic compound according to claim 2.

10. A display apparatus comprising:
the light-emitting device according to claim 9, and at least one of a transistor and a substrate.

11. An electronic appliance comprising;
the display apparatus according to claim 10, and at least one of a sensor portion, an input portion, and a communication portion.

12. A light-emitting device comprising the organic compound according to claim 3.

13. A display apparatus comprising:
the light-emitting device according to claim 12, and at least one of a transistor and a substrate.

14. An electronic appliance comprising;
the display apparatus according to claim 13, and at least one of a sensor portion, an input portion, and a communication portion.
